(19) United States
(12) Patent Application Publication
(10) Pub. No.: US 2026/0284071 A1
(43) Pub. Date: Sep. 24, 2026

(54) CONTROLLING HOMEOSTATIC REGULATORY CIRCUITRY IN HYPOTHALAMUS

(71) Applicant: The Johns Hopkins University, Baltimore, MD (US)

(72) Inventors: Seth Blackshaw, Baltimore, MD (US); Sooyeon Yoo, Seoul (KR); Leighton H. Duncan, Baltimore, MD (US)

(21) Appl. No.: 18/705,720

(22) PCT Filed: Oct. 31, 2022
(86) PCT No.: PCT/US2022/079002

§ 371 Date: Apr. 29, 2024

Related U.S. Application Data
(60) Provisional application No. 63/273,854, filed on Oct. 29, 2021.

(51) Int. Cl. A61K 31/55; A61K 31/4436; A61K 31/497; A61K 31/538; A61K 31/7105; A61K 35/76; A61P 25/28; C12N 15/86

(52) U.S. Cl. CPC A61K 31/55; A61K 31/4436; A61K 31/497; A61K 31/538; A61K 31/7105; A61K 35/76; A61P 25/28; C12N 15/86; C12N 2750/14143

(57) ABSTRACT

Provided herein are, inter alia, methods, compositions and kits for treating a hypothalamic-regulated behavior Also included herein are kits for treating a hypothalamic-regulated behavior.

The present application claims the benefit of U.S. provisional application No. 63/273,854 filed Oct. 29, 2021, which is incorporated herein by reference in its entirety.

### STATEMENT AS TO FEDERALLY SPONSORED RESEARCH

This invention was made with government support under grant R01DK108230 awarded by the National Institutes of Health. The government has certain rights in the invention.

### SEQUENCE LISTING STATEMENT

The instant application contains a Sequence Listing which has been submitted in XML format and is hereby incorporated by reference in its entirety. Said XML copy, created on Dec. 15, 2022, is named 348358_12401_SL.xml and is 51,813 bytes in size.

### BACKGROUND

New compositions and methods for treating a hypothalamic-regulated behaviors are needed.

### BRIEF SUMMARY

Provided herein, inter alia, are methods and compositions for preventing or treating a hypothalamic-regulated behavior in a subject, the method including administering an effective amount of an agent to the subject, wherein the agent decreases the activity or expression of a nuclear factor I (NFI) gene or transcription factor (e.g., a human NFI gene or transcription factor). In embodiments, the hypothalamic-regulated behavior comprises obesity, type II diabetes, a sleep disorder, hypertension, anorexia nervosa, congenital hypothyroidism, neuropsychiatric disorders linked to dysregulation of cortisol, depression, or post-traumatic stress disorder.

In embodiments, the method includes administering an agent, and the agent includes a small molecule, an antibody or fragment thereof, a polypeptide, a nucleic acid molecule, an adeno-associated virus (AAV), protein degraders, or any combination thereof. For example, the nucleic acid molecule includes small interfering RNA (siRNA), micro RNA (miRNA), RNA interference (RNAi), or any combination thereof.

In embodiments, the agent includes an adeno-associated virus, where the AAV includes AAV1, AAV2, AAV4, AAV5, AAV6, AAV7, AAV8, AAV9, or AAVDJ8.

In embodiments, the method includes administering an agent, where the agent is a small molecule. In certain embodiments, a small molecule comprises ABC99, SAG (smoothened agonist), LY411575 (gamma secretase inhibitor/Notch antagonist) or combinations thereof.

In embodiments, the method further comprises decreasing the inhibition Shh signaling, and/or Wnt signaling. For example, the method further includes administering a second agent that activates the Shh signaling and/or Wnt signaling. In embodiments, the second agent includes an Shh signaling agonist or a Wnt signaling agonist. In certain embodiments, an Shh signaling agonist comprises SAG (smoothened activator). In certain embodiments, a Wnt signaling agonist comprises ABC99 (N-hydroxyhydantoin carbamate inhibitor).

In other embodiments, the second agent comprises a small molecule. For example, the small molecule includes 7-(4-Chlorobenzyl)-1,3-dioxohexahydroimidazo[1,5-a]pyrazin-2(3H)-yl 2,3-dihydro-4Hbenzo[b][1,4]oxazine-4-carboxylate.

In other embodiments, the method further includes administering an agent that targets Krüppel-like Factor 2 (Klf2), Krüppel-like Factor 2 (Klf3), V-maf musculoaponeurotic fibrosarcoma oncogene homolog B (Mafb), or combinations thereof.

In other examples the method further includes administering an agent that targets Notch homolog 1, translocation-associated (Notch1), Transforming Growth Factor Beta 2 (TGFβ2), Bone Morphogenetic Protein 7 (Bmp7), or combinations thereof. In certain embodiments, an agent that targets Notch comprises LY411575 (gamma secretase inhibitor/Notch antagonist).

In embodiments, the method for preventing or treating a hypothalamic-regulated behavior in a subject includes treating a mammal, e.g., a human.

In embodiments, the effective amount of the agent may be from about 0.001 mg/kg to about 250 mg/kg body weight. In examples, the agent (or the composition comprising the agent) can be administered systemically or locally). In embodiments, the agent (or the composition comprising the agent) further includes a pharmaceutically acceptable carrier.

In aspects, provided herein is a method for enhancing neurogenic competence in an glial cell in a subject, where the method includes administering an effective amount of an agent to the subject, wherein the agent wherein the agent decreases the activity or expression of a nuclear factor I (NFI) gene. In embodiments, the neurogenic competence includes outward radial migration, maturation, or integration into existing hypothalamic circuitry.

In embodiments, the glial cell includes a tanycyte cell or an astrocyte.

In embodiments, the method includes administering an agent, and the agent includes a small molecule, an antibody or fragment thereof, a polypeptide, a nucleic acid molecule, an adeno-associated virus (AAV), protein degraders, or any combination thereof. For example, the nucleic acid molecule includes small interfering RNA (siRNA), micro RNA (miRNA), RNA interference (RNAi), or any combination thereof.

In embodiments, the agent includes an adeno-associated virus (AAV), where the AAV includes AAV1, AAV2, AAV4, AAV5, AAV6, AAV7, AAV8, AAV9, or AAVDJ8.

In certain embodiments, the AAV comprises one or more nucleic acid molecules having at least about 70% (such as at least about 75%, 80%, 85%, 90%, 91%, 92%, 93%, 94%, 95%, 96%, 97%, 98%, 99%, or greater) sequence identity to SEQ ID NOS: 1-26.

In certain embodiments, the AAV comprises one or more nucleic acid molecules comprising SEQ ID NOS: 1-26.

In certain embodiments, the agent comprises comprise a nucleic acid molecule having at least about 70% (such as at least about 75%, 80%, 85%, 90%, 91%, 92%, 93%, 94%, 95%, 96%, 97%, 98%, 99%, or greater) sequence identity to SEQ ID NOS: 1-26.

In certain embodiments, the agent comprises comprise a nucleic acid molecule comprising a nucleic acid sequence of any one or more of SEQ ID NOS: 1-26.

In certain embodiments, a synthetic construct comprising a nucleic acid sequence of any one of SEQ ID NOs: 1-26 or combinations thereof.

In certain embodiments, an isolated cell comprises a nucleic acid molecule having a sequence identity of at least 75% to at least one of SEQ ID NOS: 1-26. In certain embodiments, a the nucleic acid molecule comprises any one of SEQ ID NOS: 1-26.

In certain embodiments, an isolated cell comprises an adeno-associated virus (AAV) comprising a nucleic acid molecule having a sequence identity of at least 75% to at least one of SEQ ID NOS: 1-26. In certain embodiments, the nucleic acid molecule comprises any one of SEQ ID NOS: 1-26.

In certain embodiments, the isolated cell comprises stem cells, cord blood cells, adult stem cells, mesenchymal stem cells, induced pluripotent stem cells, autologous cells, autologous stem cells, bone marrow cells, hematopoietic cells, hematopoietic stem cells, somatic cells, germ line cells, differentiated cells, somatic stem cells, embryonic stem cells, autologous cells, allogeneic cells, haplotype matched cells, haplotype mismatched cells, haplo-identical cells, xenogeneic cells, cell lines or combinations thereof.

In certain embodiments, a method of treating a hypothalamic-regulated behavior in a subject, the method comprising, administering an effective amount of an agent to the subject, wherein the agent comprises an Shh signaling agonist, a Wnt signaling agonist, an adeno-associated virus (AAV) or combinations thereof. In certain embodiments, the AAV comprises one or more comprises one or more nucleic acid molecules comprising SEQ ID NOS: 1-26.

In embodiments, the method includes administering an agent, where the agent is a small molecule.

In embodiments, the method further comprises decreasing the inhibition Shh signaling, and/or Wnt signaling. For example, the method further includes administering a second agent that activates the Shh signaling and/or Wnt signaling. In embodiments, the second agent includes an Shh signaling agonist or a Wnt signaling agonist.

In other embodiments, the second agent comprises a small molecule. For example, the small molecule includes 7-(4-Chlorobenzyl)-1,3-dioxohexahydroimidazo[1,5-a]pyrazin-2(3H)-yl 2,3-dihydro-4Hbenzo[b][1,4]oxazine-4-carboxylate.

We also have found that that SAG-mediated pharmacological activation of Shh signaling in juvenile mice (P10-12) leads to an increase, including significant increases, in the number of tanycyte-derived neurons. While not being bound by any theory, this does not appear to be due to stimulating tanycyte proliferation, but rather to directly promoting differentiation of tanycytes into neurons and/or promoting the survival of newly generated tanycyte-derived neurons.

We have further found that that ABC99-mediated activation of Wnt signaling in juvenile mice (P10-12) also promotes generation of tanycyte-derived cells in hypothalamic parenchyma that resemble neurons. This effect is significant, but can be smaller than the effects of SAG.

We also have determined that intracerebroventricular delivery of the AAV1 serotype can selectively and efficiently infect tanycytes. It was further found that the AAV5 and AAV9 serotypes inefficiently infect tancytyes, while AAV2, AAV6, AAV8, and AAV7m8 do not detectably infect tanycytes. This can be important to induce tanycyte-derived neurogenesis, and to guide the differentiation of tanycyte-derived neurons towards therapeutically relevant neuronal subtypes controlling homeostatic processes such as for example sleep/wake regulation, food intake, energy consumption, and stress hormone release.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (includes FIGS. 1A-1O) are data showing Nfia/b/x suppress proliferation and neurogenesis in tanycytes of neonatal mice. FIG. 1A are data showing the expression of Nfia/b/x in GFP+ tanycytes isolated from Rax:GFP mice (19) compared to the GFP-negative cells in adult hypothalamus. The tanycyte-specific marker Rax and the neuronal marker Npy are enriched in GFP+ and GFP− cells, respectively. FIG. 1B is an image showing the distribution of Nfia/b/x protein in Rax-GFP+ tanycytes. FIG. 1C is a schematic of mouse lines used in this study. FIG. 1D is a schematic of a genetic approach for simultaneous tanycyte-specific disruption of Nfia/b/x and reporter gene labeling of tanycytes and tanycyte-derived cells using tamoxifen-dependent activation of CreER. FIG. 1E are images showing the induction of proliferation and neurogenesis in NFI-deficient tanycytes by P17. FIG. 1F are graphs showing the quantification of proliferation and neurogenesis in the ventricular zone (VZ) and hypothalamic parenchyma (HP) at P17 (n=3-5 mice). FIG. 1G are images showing that in NFI TKO mice by P45, NeuN/neurofilament M-positive tanycyte-derived neurons migrate into the parenchyma of the arcuate nucleus (ArcN) and dorsomedial hypothalamus (DMH), with a small number of neurons remaining in the subventricular zone (yellow arrowheads). Enlarged image of parenchymal tanycyte-derived neurons in g with the orthogonal view showing co-staining within the cell. FIG. 1H is a graph showing that a substantially increased numbers of NeuN+/GFP+ tanycyte-derived neurons are observed in NFI TKO mice in ArcN and DMH relative to wildtype controls, but comparable numbers of neurons are observed in median eminence (ME) and ventromedial hypothalamus (VMH) (n=2-3 mice). FIG. 1I is a graph showing that the number of GFP+ tanycytes is reduced in NFI-deficient mice at P45, and ectopic neurons are seen in the VZ (n=2-3 mice). FIG. 1J is a schematic for i.e.v. delivery of AAV-Cre and analysis of Nfia/b/x loss of function in P78 mice. FIG. 1K are images showing that AAV-Cre induces Sun1-GFP expression in tanycytes in CAG-lsl-Sun1-GFP control mice at P78 (k inset shows alpha tanycytes). FIG. 1L are images showing that AAV-Cre induces proliferation in alpha tanycytes (shown in inset 1) of Nfia^{lox/lox}; Nfib^{lox/lox}; Nfix^{lox/lox}; CAG-lsl-SunU-GFP mice. FIG. 1M is a graph showing the quantification of GFP+/EdU+ cells in VZ and HP in NFI-deficient mice (n=4-5 mice). FIG. 1N is a graph showing the number of GFP+ cells in VZ and HP in control and NFI-deficient mice. FIG. 1O is a graph showing the percentage of GFP+VZ cells in the alpha tanycyte region labeled by Ki67 and EdU in NFI-deficient mice (n=4-5 mice). Scale bars: FIGS. 1B, 1E, 1K and 1L=100 μm; G=50 μm; FIGS. 1G, 1K and 1L=25 μm.

FIG. 2 (includes FIGS. 2A-2J) are data showing single-cell RNA-Seq analysis of control and NFI-deficient tanycytes. FIG. 2A is an aggregate UMAP plot of scRNA-Seq data from control and NFI-deficient GFP+ tanycytes and tanycyte-derived cells isolated at P8, P17 and P45. Cell types are indicated by color shading. FIG. 2B are images of distribution of cell type-specific marker expression on aggregate UMAP plot. FIG. 2C are images of distribution of cells by age and genotype on aggregate UMAP plot. FIG. 2D are data showing the percentage of each cell type by age and genotype. FIG. 2E is a dot plot showing genes differentially expressed in Nfia/b/x-deficient alpha2 tanycytes. FIG. 2F are images of Hiplex analysis for tanycyte subtype-specific markers and enhanced Shh expression in a subset of Pdzph1+ alpha2 tanycytes. Necab1+ alpha1 tanycytes and Tm4sf1+ ependymal cells are shown for reference. FIG. 2G are images of RNA velocity analysis indicating differentiation trajectories in tanycytes and tanycyte-derived cells. Insets highlight proliferating tanycytes and tanycyte-derived neurons. FIG. 2H are images showing pseudotime analysis of differential gene expression in alpha2 tanycytes, proliferating tanycytes and tanycyte-derived neurons. FIG. 2I is a heatmap showing differentially expressed genes over the course of tanycyte-derived neurogenesis. FIG. 2J are graphs of Gene Ontology (GO) analysis of differentially expressed genes in I, with enrichment shown at −log 10 P-value. Scale bars: F=100 μm. tany=tanycytes.

FIG. 3 (includes FIGS. 3A-31) are data showing that single-cell ATAC-Seq analysis of P8 wildtype and Nfia/b/x-deficient tanycytes demonstrates derepression of Shh and increased Wnt signaling. FIG. 3A is an aggregate UMAP plot of scATAC-Seq data from control and NFI-deficient GFP+ tanycytes and tanycyte-derived cells isolated at P8. Cell types are indicated by color shading. FIG. 3B is an image of the distribution of cell types shown for control and Nfia/b/x-deficient GFP+ cells at P8. FIG. 3C are images of the distribution of accessible consensus NFI motif shown for control and Nfia/b/x-deficient GFP+ cells. FIG. 3D are images showing transcription factor binding motifs selectively enriched and depleted in control and Nfia/b/x-deficient alpha2 tanycytes. FIG. 3E is data showing the consensus NFI footprint distribution in control and Nfia/b/x-deficient alpha2 tanycytes. FIG. 3F are data showing the integration of scATAC-Seq and scRNA-Seq data to identify differentially expressed genes in alpha2 tanycytes that are directly regulated by Nfia/b/x. FIG. 3G are data of Gene Ontology analysis of Nfia/b/x-regulated genes expressed in alpha2 tanycytes. FIG. 3H are data showing that Shh is directly repressed by Nfia/b/x in alpha2 tanycytes. FIG. 3I is a schematic of the summary of Nfia/b/x action in alpha2 tanycytes.

FIG. 4 (includes FIGS. 4A-4D) are data showing that Shh and Wnt signaling stimulate proliferation and neurogenesis in Nfia/b/x-deficient tanycytes. FIG. 4A are images showing that the expression of Shh, Notum and Sulf1 on aggregate UMAP plot. FIG. 4B is a dot plot showing expression level of Shh and Wnt pathway genes in each cluster. FIG. 4C is an image of the Shh inhibition by intraperitoneal (i.p.) cyclopamine inhibits neurogenesis in Nfia/b/x-deficient mice. FIG. 4D are data of the activation of Wnt signaling by inhibition of Notum via i.p. ABC99 induces proliferation of alpha tanycytes, and increased numbers of GFP+ proliferating cells in VZ and HP. VZ, ventricular zone; HP, hypothalamic parenchyma; TDCs, tanycyte-derived cells. Scale bars: 4C and 4D=100 μm; 4D(insets)=25 μm.

FIG. 5 (includes FIGS. 5A-5J) are data showing the identification of selective markers of tanycyte-derived neurons. FIG. 5A is a UMAP plot showing major clusters of tanycyte-derived neuronal subset, separated by age and genotype. FIG. 5B is a dot plot showing major subtype-specific markers of tanycyte-derived neurons. FIG. 5C is an image of an RNA velocity analysis which indicated differentiation trajectories for tanycyte-derived neurons. FIG. 5D are images of SmfISH analysis and FIG. 5E are immunohistochemistry images which demonstrated the expression of Th and Lhx6 in Egfp+ tanycyte-derived neurons in Nfia/b/x-deficient mice. FIG. 5F are images of SmfJSH analysis of Gal, Gad1 and Th in Egfp+ tanycyte-derived neurons in Nfia/b/x-deficient mice. FIG. 5G are images of Cart, Agrp, Slc32a1 and Th expression in Egfp+ tanycyte-derived neurons in Nfia/b/x-deficient mice. FIG. 5H are images of Nr5a1 and Slc17a6 expression in Egfp+ tanycyte-derived neurons in Nfia/b/x-deficient mice. All insets are enlarged images of examples of colocalization (white boxes in FIG. 5D, FIG. 5E, FIG. 5F, FIG. 5G, and FIG. 5H). FIG. 5I are images of pStat3 staining 45 minutes after i.p. administration of 3 mg/kg leptin in Nfia/b/x-deficient mice (n=3 mice). Arrows indicate GFP+/pStat3+ tanycyte-derived neurons. Insets show higher magnification images in DMH (i) and ArcN (i′). FIG. 5J is a bar graph showing the fraction of pStat3-positive tanycyte-derived neurons in VZ and HP after leptin administration. pStat3 was not induced in saline-injected mice (n=2 mice). VZ, ventricular zone; HP, hypothalamic parenchyma. Scale bars: D, F, G, H=50 μm; insets in FIG. 5D, FIG. 5F, FIG. 5G, FIG. 5H=10 μm; FIG. 5E, FIG. 5H, FIG. 5I=100 μm; high magnification images in E=20 μm.

FIG. 6 (includes FIGS. 6A-6L) are data showing that Nfia/b/x-deficient tanycytes differentiate into neurons, integrate into hypothalamic neural circuitry, and respond to physiological stimuli. FIG. 6A are images showing low and high magnification confocal images showing two biocytin-filled GFP+ recorded cells (white arrows) in an NFI TKO brain slice stained with NeuN. FIG. 6B are example responses of tanycyte-derived cells to depolarizing current steps. FIG. 6C are images showing the proportion of tanycyte-derived neurons among tested tanycyte-derived cells in young (left) and adult (right) mice. FIG. 6D are data showing representative average responses to hyperpolarizing current steps. FIG. 6E is a bar graph showing summary graphs of input resistance (young control neurons, 18 cells from 5 mice, 1,101±89 MΩ, young tanycyte-derived neurons, 17 cells from 5 mice, 1,546±176 MΩ, p=0.0238, Mann-Whitney U test; adult control neurons, 16 cells from 6 mice, 1,369±132 MΩ, adult tanycyte-derived neurons, 18 cells from 6 mice, 1,477±119 MΩ, p=0.4777, Mann-Whitney U test). FIG. 6F are representative voltage traces recorded from control and tanycyte-derived neurons from young and adult mice evoked by 10-40 pA depolarizing current steps as indicated. FIG. 6G are line graphs showing the current-spike frequency relationships measured from control and tanycyte-derived neurons from young mice (top) and adult mice (bottom). The current-frequency was significantly different between tanycyte-derived and control neurons at both ages (Young mice: control neurons, 14 cells from 4 mice; tanycyte-derived neurons, 13 cells from 4 mice, p<0.0001, two-way ANOVA; Adult mice: control neurons, 16 cells from 6 mice, tanycyte-derived neurons, 18 cells from 6 mice, p=0.0001, two-way ANOVA). FIG. 6H are representative traces of spontaneous postsynaptic currents (sPSCs). FIG. 6I is a bar graph showing summary graphs of sPSC frequency (young control neurons, 14 cells from 4 mice, 2.85±0.74 Hz, young tanycyte-derived neurons, 13 cells from 4 mice, 4.07±1.93 Hz, p=0.2983, Mann-Whitney U test; adult control neurons, 16 cells from 6 mice, 7.56±1.82 Hz, adult tanycyte-derived neurons, 18 cells from 6 mice, 2.98±0.54 Hz, p=0.0324, Mann-Whitney U test). FIG. 6J is a line graph showing the positive correlation between the distance from the tanycytic layer for each tanycyte-derived neuron and its sPSC frequency in both young (left, 12 cells from 4 mice, p=0.0234, Spearman's Rho correlation) and adult (right, 18 cells from 6 mice, p=0.0451, Spearman's Rho correlation) mice. FIG. 6K are images showing 4 hr heat stress (38° C.) selectively induced c-fos expression in tanycyte-derived neurons in DMH (higher magnification inset shown in right). FIG. 6L are bar graphs showing the fraction of c-fos-positive tanycyte-derived neurons in VZ and HP of DMH, and fraction of all c-fos-positive and negative neurons in DMH (n=3 mice). ArcN=arcuate nucleus, DMH=dorsomedial hypothalamus, HP=hypothalamic parenchyma, ME=median eminence, PH=posterior hypothalamus, VZ=ventricular zone. Scale bars: A. Left=50 μm, right 20 μm, K=100 μm (inset=20 μm).

FIG. 7 (includes FIGS. 7A-7D) are data showing time course of tamoxifen-dependent loss of Nfia/b/x protein expression and induction of proliferation in Nfia/b/x mutant mice. FIG. 7A is a schematic showing the loss of Nfia/b/x immunoreactivity and induction of BrdU labeling following tamoxifen treatment of Rax-CreER; Nfialox/lox; Nfiblox/lox; Nfixlox/lox; CAG-Isl-Sun1-GFP mice at P6, P8 and P10. FIG. 7B are images showing the induction of proliferation occurs in alpha tanycytes before being detectable in beta tanycytes. FIG. 7C are images showing that the alpha tanycytic ventricular zone thickens at P12 and Ki67+ cells are localized in the most superficial layer while immunoreactivity to HuC/D and/or NeuN is detected at the layer closest to the parenchyma. Antibodies to HuC/D and NeuN were combined for this analysis. FIG. 7D are images showing the near-complete, tanycyte-specific loss of Nfia/b/x immunoreactivity by P17. Scale bars: A, D=100 μm; B,C=20 μm.

FIG. 8 (includes FIGS. 8A-8D) are data showing that tamoxifen-dependent Cre activation does not effectively induce proliferation in Nfia/b/x mutant mice at P12. FIG. 8A is a schematic for tamoxifen-dependent disruption of Nfia/b/x function in Rax-CreER; Nfialox/lox; Nfiblox/lox; Nfixlox/lox; CAG-lsl-Sun1-GFP mice at P7, P10, and P12. FIG. 8B are images showing induction of proliferation and neurogenesis by Nfia/b/x deletion at P7, P10, and P12. FIG. 8C is a bar graph showing the mosaic loss of Nfia/b/x immunostaining following Cre activation at P12. FIG. 8D are images showing BrdU labeling in ventricular zone (VZ) and hypothalamic parenchyma (HP) following tamoxifen treatment at P7 (n=3 mice), P10 (n=4 mice) and P12 (n=2-3 mice). Scale bars: B=100 μm, D=25 μm. Abbreviations: 3V=third ventricle, DMH=dorsomedial hypothalamus, ME=median eminence.

FIG. 9 (includes FIGS. 9A-9C) are data showing that AAV-Cre-mediated deletion of NFI genes induced tanycyte proliferation in adult mice. FIG. 9A is a schematic showing Cre-dependent AAV-based deletion strategy. AAV1-Cre-mCherry was injected into the lateral ventricles of Nfia^{lox/lox}; Nfib^{lox/lox}; Nfix^{lox/lox}; CAG-lsl-Sun-GFP (Nfia/b/xf/f; Sun1-GFP) or CAG-lsl-Sun1-GFP (Sun1-GFP, as controls) mice at P60, followed by BrdU infusion over 2 weeks using an osmotic minipump. FIGS. 9B and 9C are images showing confirmation of viral infection with immunolabeling for mCherry and GFP induction in the ventricular layer of both controls (FIG. 9B) and NFI knockout mice (FIG. 9C). A subset of parenchymal GFP+ cells were immunoreactive for HuC/D and NeuN (yellow arrows in inset 1), but BrdU-negative. Only NFI knockout mice show BrdU and GFP double-positive cells in the tanycytic layer (white arrows in inset 2). Induction of proliferation was confirmed with co-immunostaining for Ki67 and GFP in the tanycytic layer (yellow arrows in inset 3). Scale bars: B, C=100 μm; inset 1, 3=50 μm; inset 2=25 μm.

FIG. 10 (includes FIGS. 10A-10C) are data showing clustering of GFP-positive cells at different ages. P8 (FIG. 10A), P17 (FIG. 10B), P45 (FIG. 10C).

FIG. 11 are images of heatmaps showing differentially expressed genes in each tanycytes subtype. Major changes in gene expression observed in TKO alpha2 tanycytes. Up-regulated Notum and Shh expression in alpha2 tanycytes is highlighted (Red arrows).

FIG. 12 (includes FIGS. 12A-12C) are data showing hypothalamic neuronal distribution and comparison analysis with a published scRNA-Seq data from ArcN neurons. FIG. 12A is an image showing that no obvious change is observed in the overall distribution of hypothalamic neurons in Nfia/b/x-deficient mice by Hiplex RNAscope analysis. FIG. 12B are images showing a LIGER-based comparison of P8, P17 and P45 tanycyte-derived neurons with scRNA-Seq data from normal and low fat chow-fed mice from ArcN (Campbell, et al. 2017), with tanycyte-derived neurons and ArcN neurons plotted separately. FIG. 12C are data showing an alluvial plot indicating relationships between identified clusters of tanycyte-derived neurons (this study) and ArcN neurons (from Campbell, et al. 2017). Scale bars: A=100 μm.

FIG. 13 (includes FIGS. 13A-13E) are data showing the morphology of biocytin-filled tanycyte-derived and control neurons and non-neuronal tanycyte-derived cells. FIG. 13A are images showing three examples of biocytin-filled tanycyte-derived and control neurons (magenta). GFP+ tanycyte-derived neurons and GFP− control neurons were recorded from TKO brain slices. FIGS. 13B and 13C are images showing two tanycyte-derived cells in the hypothalamic parenchyma (left images, DIC images) of young Nfla/b/x-deficient mice (FIG. 13B: P18 mouse, FIG. 13C: P16 mouse) that did not fire action potentials in response to step depolarizations of current (right). Low (FIG. 13D) and high (FIG. 13E) magnification confocal images of the cell shown in FIG. 13C visualized by biocytin-streptavidin staining. This cell expressed GFP but showed glial cell-like morphology. Scale bars: FIGS. 13A-D=50 μm, FIG. 13E=10 μm.

FIG. 14 (includes FIGS. 14A-14D) are data showing that tanycytes in the tanycytic layer are non-spiking cells in NFIa/b/x TKO mice. FIGS. 14A-D. DIC and GFP fluorescence images of four cells located in the tanycytic layer and their membrane potential responses to depolarizing and hyperpolarizing current steps. Cells were recorded from P18 (FIG. 14A) and P42 (FIGS. 14B-D) NFIa/b/x TKO mice. Note that these cells did not fire action potentials to depolarizing current steps. Scale bars: FIGS. 14A-D=50 μm.

FIG. 15 (includes FIGS. 15A-15E) are data showing that tanycyte-derived neurons fire spontaneous action potentials and show increased firing with age. FIG. 15A are data showing examples of recorded cells showing spontaneous action potentials (sAPs) at their resting potentials. FIG. 15B are data showing the proportion of neurons displaying sAPs is higher in control neurons than tanycyte-derived neurons in young mice (p=0.0437, Fisher's exact test) whereas there is no difference in adult mice (p=0.3269, Fisher's exact test). FIG. 15C are graphs showing the current-spike frequency relationship plots for individual young control neurons (14 cells from 4 mice), young tanycyte-derived neurons (13 cells from 4 mice), adult control neurons (16 cells from 6 mice), and adult tanycyte-derived neurons (18 cells from 6 mice). The summary data is shown in FIG. 6G. One neuron (plotted in bold orange) did not fire action potentials to 10-40 pA current injections, but generated action potentials following a 340 pA current injection and was therefore categorized as a tanycyte-derived neuron. FIGS. 15E and 15D., FIG. 15E is a graph showing current-spike frequency relationship plots showing differences in spike frequency of control neurons (FIG. 15D) or tanycyte-derived neurons (FIG. 15E) between young and adult mice. Both control neurons (p=0.0304, Two-way ANOVA) and tanycyte-derived neurons (p=0.02977, Two-way ANOVA) showed significantly higher spike frequency in cells from adult mice compared to cells from young mice.

FIG. 16 shows results of a model identifying extrinsic factors controlling tanycyte-derived neurogenesis in the hypothalamus and results shown with activation of Shh signaling with SAG and activation of Wnt signaling with ABC99.

FIG. 17 shows results of Shh pathway activation promotes tanycyte-derived neurogenesis in neonatal hypothalamus.

FIG. 18 shows results of Wnt pathway activation stimulates tanycyte transdifferentiation into hypothalamic parenchymal cells.

FIG. 19 shows results of elective targeting of hypothalamic tanycytes with AAV1.

FIG. 20 shows results of selective targeting of hypothalamic tanycytes with AAV1.

### DETAILED DESCRIPTION

Provided herein are, inter alia, methods, compositions and kits for treating and preventing hypothalamic-regulated behavior in a subject, the method including administering an effective amount of an agent to the subject, wherein the agent wherein the agent decreases the activity or expression of a nuclear factor I (NFI) gene or transcription factor (e.g., a human NFI gene or transcription factor). In embodiments, the hypothalamic-regulated behavior comprises obesity, type II diabetes, a sleep disorder, hypertension, anorexia nervosa, congenital hypothyroidism, neuropsychiatric disorders linked to dysregulation of cortisol, depression, or post-traumatic stress disorder. In other aspects, provided herein is a method for enhancing neurogenic competence in an glial cell in a subject, where the method includes administering an effective amount of an agent to the subject, wherein the agent wherein the agent decreases the activity or expression of a nuclear factor I (NFI) gene. For example, the neurogenic competence includes outward radial migration, maturation, or integration into existing hypothalamic circuitry

Hypothalamic tanycytes, radial glial cells that share many features with neuronal progenitors, can generate small numbers of neurons in the postnatal hypothalamus, but the identity of these neurons and the molecular mechanisms that control tanycyte-derived neurogenesis are unknown. As provided herein, tanycyte-specific disruption of the NFI family of transcription factors (Nfia/b/x) stimulates proliferation and tanycyte-derived neurogenesis. Single-cell RNA- and ATAC-Seq analysis reveals that NFI factors repress Shh and Wnt signaling in tanycytes, and small molecule inhibition of these pathways blocks proliferation and tanycyte-derived neurogenesis in Nfia/b/x-deficient mice. Moreover, Nfia/b/x-deficient tanycytes gave rise to multiple mediobasal hypothalamic neuronal subtypes that can mature, integrate into hypothalamic circuitry, and selectively respond to changes in internal states. These findings identify molecular mechanisms controlling tanycyte-derived neurogenesis that can be targeted to selectively remodel hypothalamic neural circuitry controlling homeostatic physiological processes.

Hypothalamic tanycytes are radial glial cells that line the ventricular walls of the mediobasal third ventricle (1, 2). Tanycytes are subdivided into alpha1, alpha2, beta1 and beta2 subtypes based on dorso-ventral position, morphology and gene expression profile, and closely resemble neural progenitors in morphology and gene expression profile. Tanycytes have been reported to generate small numbers of neurons and glia in the postnatal period, although at much lower levels than in more extensively characterized sites of ongoing neurogenesis such as the subventricular zone of the lateral ventricles or the subgranular zone of the dentate gyrus (3-6). While tanycyte-derived newborn neurons may play a role in regulating a range of behaviors (3, 7, 8), levels of postnatal tanycyte-derived neurogenesis are low and virtually undetectable in adulthood. Furthermore, little is known about the molecular identity or connectivity of tanycyte-derived neurons (6, 9). A better understanding of the gene regulatory networks that control neurogenic competence in hypothalamic tanycytes would both give insight into the function of tanycyte-derived neurons and potentially identify new therapeutic approaches for modulation and repair of hypothalamic neural circuitry.

Retinal Müller glia, which closely resemble hypothalamic tanycytes in morphology and gene expression, provide valuable insight into the neurogenic potential of tanycytes (1, 3, 9, 10). Zebrafish Müller glia function as quiescent neural stem cells, and are able to regenerate every major retinal cell type following injury (11). While mammalian Müller glia effectively lack neurogenic competence, in posthatch chick they retain a limited neurogenic competence that resembles that of mammalian tanycytes (12, 13). Recent studies in retina have identified the NFI family of transcription factors Nfia/b/x as being essential negative regulators of neurogenesis in both late-stage progenitor cells and in mature mammalian Müller glia (14, 15). Moreover, like in retina, NFI factors are expressed in late-stage hypothalamic neural progenitors (16), and Nfia is necessary for hypothalamic glia specification (17). These findings raise the possibility that NFI factors may serve similar functions in the mammalian hypothalamus, namely repressing neurogenic competence in tanycytes.

To address this possibility, Nfia/b/x function was disrupted in hypothalamic tanycytes of both juvenile and adult mice. Early loss of function of NFI activity in hypothalamic tanycytes led to a robust induction of proliferation and neurogenesis, while Nfia/b/x disruption in adults led to lower levels of tanycyte-derived proliferation than are seen following neonatal loss of function. NFI loss of function activated both Shh and Wnt signaling in tanycytes, and this in turn stimulated proliferation and neurogenesis. These tanycyte-derived neurons survive, mature, migrate radially away from the ventricular zone, express molecular markers of diverse hypothalamic neuronal subtypes, and functionally integrate into hypothalamic circuitry. These findings demonstrate that hypothalamic tanycytes possess a latent neurogenic competence that is actively suppressed by NFI family transcription factors, which can be modulated to induce generation of multiple hypothalamic neuronal subtypes.

### Advantages of the Current Invention

The data herein demonstrated that tanycytes retain the ability to generate a broad range of different subtypes of hypothalamic neurons in the postnatal brain, and that this latent ability is actively repressed by NFI family transcription factors. Induction of proliferative and neurogenic competence by selective loss of function of Nfia/b/x leads to the robust generation of hypothalamic neuronal precursors that undergo outward radial migration, mature, and integrate into existing hypothalamic circuitry. Tanycyte-derived neurons respond to dietary signals, such as leptin, and heat stress. This implies that tanycyte-derived neurogenesis could modulate a broad range of hypothalamic-regulated physiological processes.

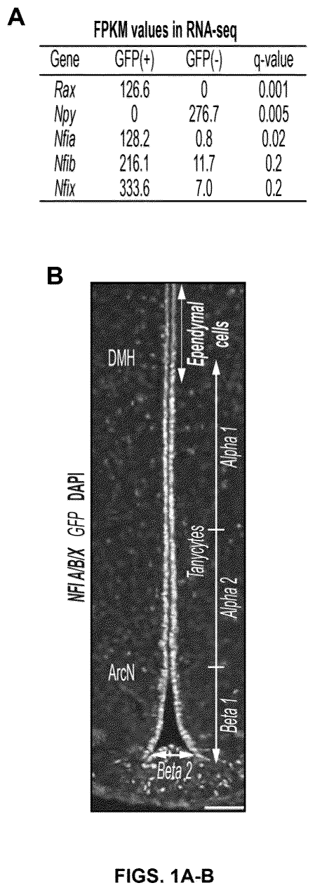

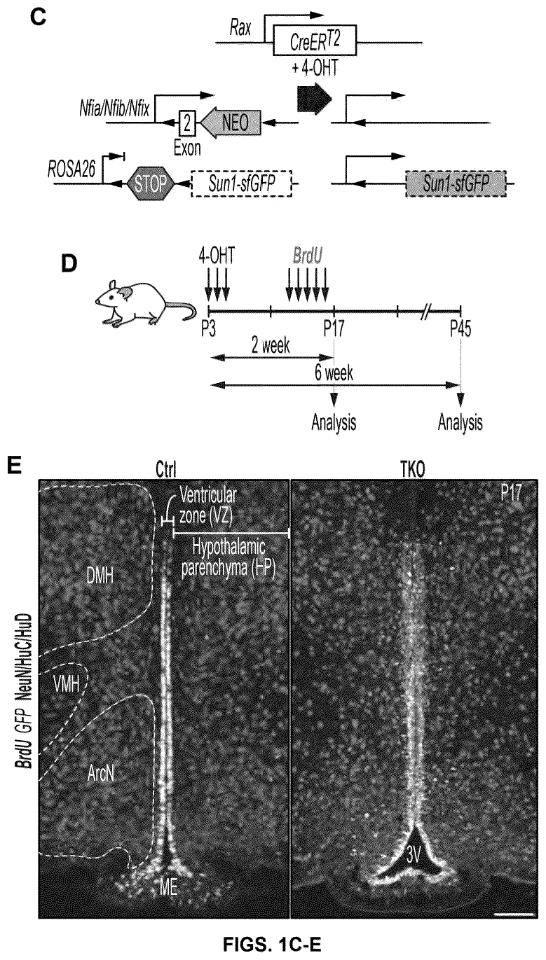

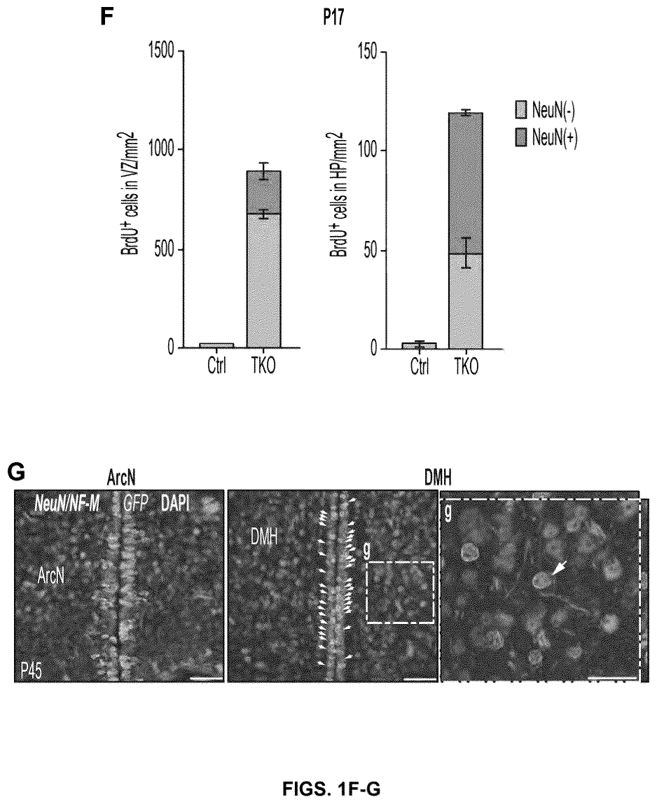

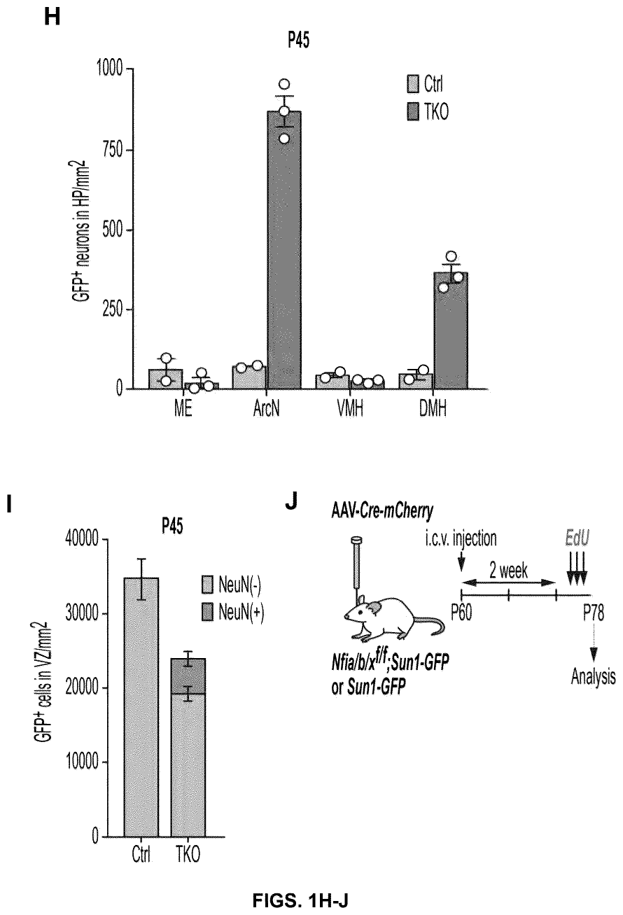

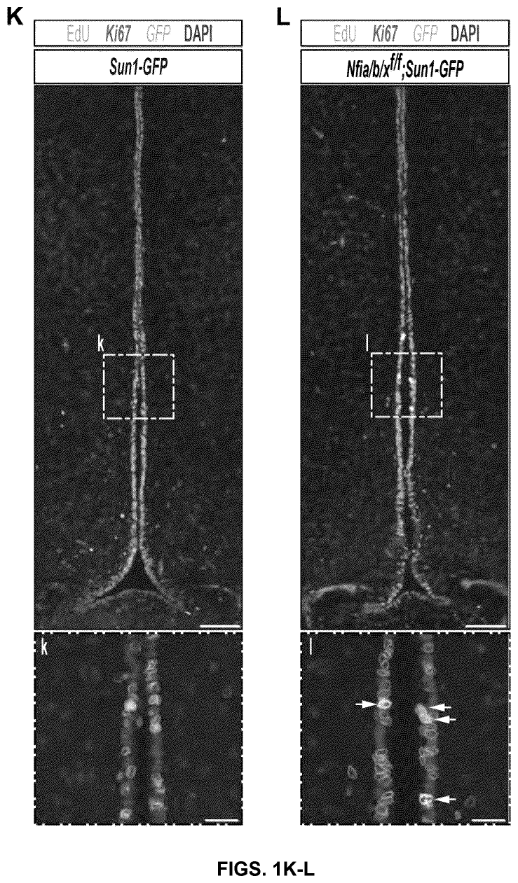

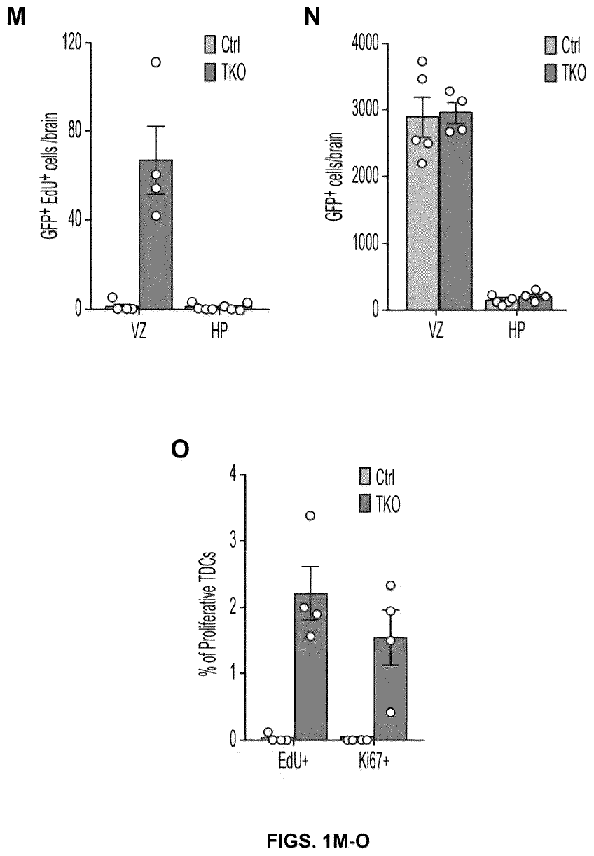

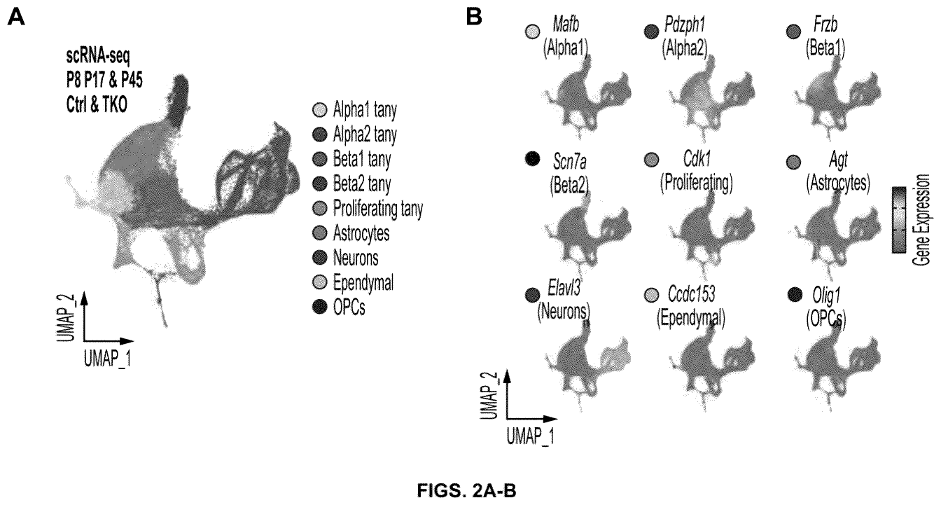

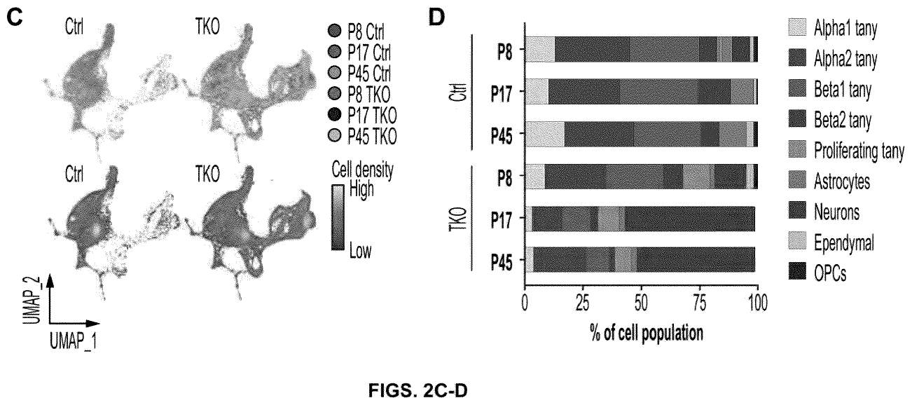

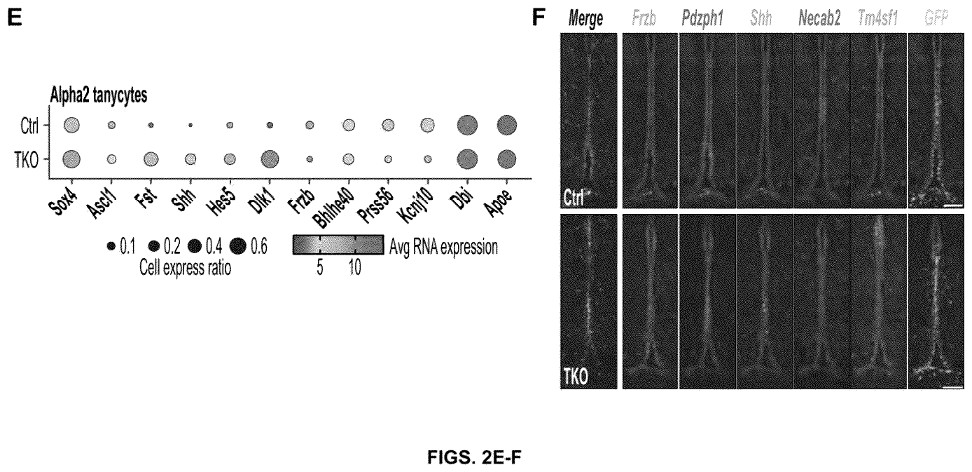

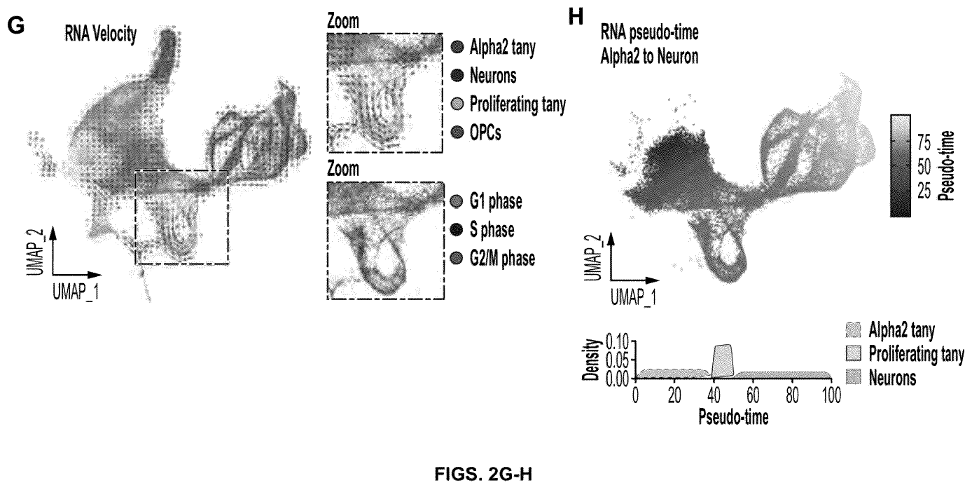

NFI factors have historically been primarily studied in the context of promoting astrocyte specification and differentiation (40, 41), and loss of function of Nfia/b/x disrupts generation of tanycyte-derived astrocytes, ependymal cells and oligodendrocyte progenitors, and downregulates expression of tanycyte-enriched genes that are also expressed in astrocytes such as Kcnj10 and Aqp4 (FIGS. 2A-2J). In addition to their role in promoting gliogenesis, however, recent studies have shown that NFI factors confer late-stage temporal identity on retinal progenitors (14, 42), allowing generation of late-born bipolar neurons and Müller glia, and decreasing proliferative and neurogenic competence. Selective loss of function of Nfia/b/x in mature Müller glia likewise induces proliferation and generation of inner retinal neurons (15), although the levels seen are lower than seen following loss of function in retinal progenitors (14). As in retina, Nfia/b/x are more strongly expressed in late than early-stage mediobasal hypothalamic progenitors (16), and adult tanycytes show substantially lower levels of proliferation following loss of function of Nfia/b/x than is seen in neonates (FIG. 1A-1O). However, the levels of proliferation and neurogenesis seen in neonatal tanycytes are much greater than those seen in Müller glia, which likely reflects the fact that mammalian Müller glia proliferate only rarely and essentially lack neurogenic competence (11), while tanycytes retain limited neurogenic competence (9). This implies that NFI factors may be part of a common gene regulatory network that represses proliferation and neurogenic competence in radial glia of the postnatal forebrain and retina.

Using scRNA and ATAC-Seq analysis, we identified multiple candidate extrinsic and intrinsic regulators of proliferative and neurogenic competence in tanycytes, many of which are known to regulate these developmental processes. We observe that loss of function of Nfia/b/x effectively regresses tanycytes to a progenitor-like state. Both Shh (43-45) and Wnt (46-48) signaling are required for progenitor proliferation and neurogenesis in embryonic tuberal hypothalamus, and the study demonstrates that they play similar roles in Nfia/b/x-deficient tanycytes. Tanycyte-specific loss of function of Nfia/b/x both downregulates Notch pathway components and upregulates the Notch inhibitor Dlk1, while also downregulating Tgfb2. Both Notch signaling and Tgfb2 promote quiescence and inhibit proliferation in retinal Müller glia and cortical astrocytes (49-52), and likely play a similar role in tanycytes.

Nfia/b/x-deficient tanycytes likewise downregulate transcription factors that are required for specification of astrocytes and Müller glia—including Sox8/9 (53, 54)—while upregulating neurogenic bHLH factors such as Ascl1 and Sox4. Ascl1 is both required for differentiation of VMH neurons (55) and also sufficient to confer neurogenic competence on retinal Müller glia (56, 57). NFI factors thus control expression of a complex network of extrinsic and intrinsic factors that regulate neurogenic competence in tanycytes, and it may be possible to further stimulate tanycyte-derived neurogenesis by modulating select components of this network.

scRNA-Seq analysis reveals that tanycyte-derived neurons arise from Ascl1+ precursors and are heterogeneous, falling into several molecularly distinct clusters. The gene expression profiles of control and Nfia/b/x-deficient tanycyte-derived neurons closely resemble one another, indicating that NFI factors are not obviously required for differentiation of individual neuronal subtypes, in contrast to their role in retina and cerebellum (14, 58, 59). However, the number of tanycyte-derived neurons in control samples drops dramatically after P8, with few detected at P45 (FIGS. 2A-2J). We observe an age-dependent decline in the number of tanycyte-derived neurons in controls, potentially in line with the high levels of cell death reported in postnatally generated hippocampal and olfactory bulb neurons (60). However, this is insufficient to account for this effect, and it may instead result from the well-established difficulties in obtaining viable, dissociated mature neurons following FACS for whole-cell scRNA-Seq analysis (61).

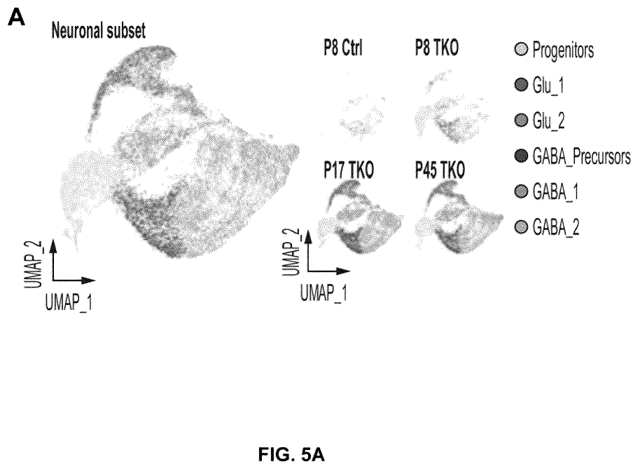

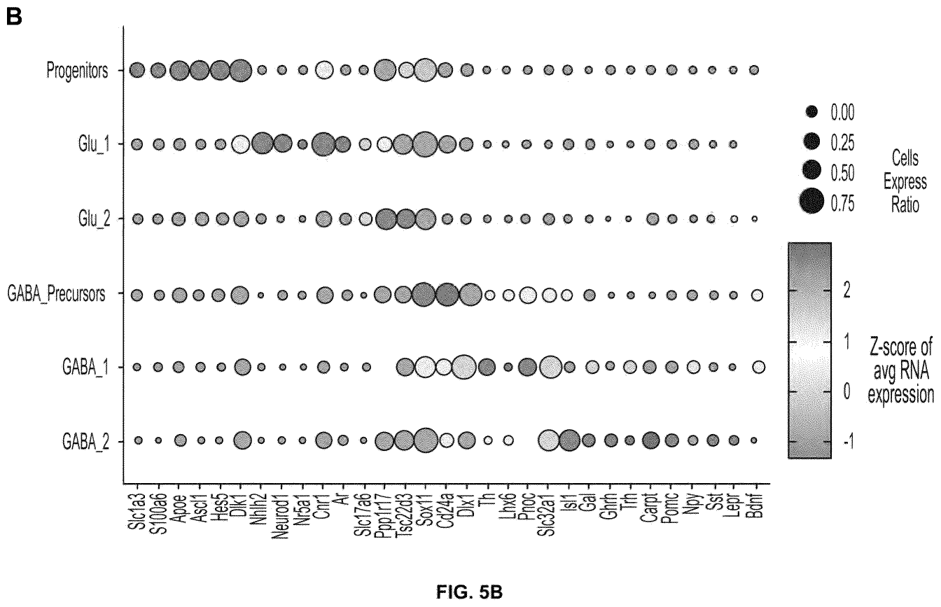

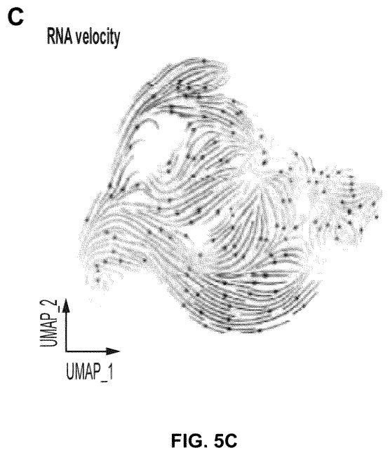

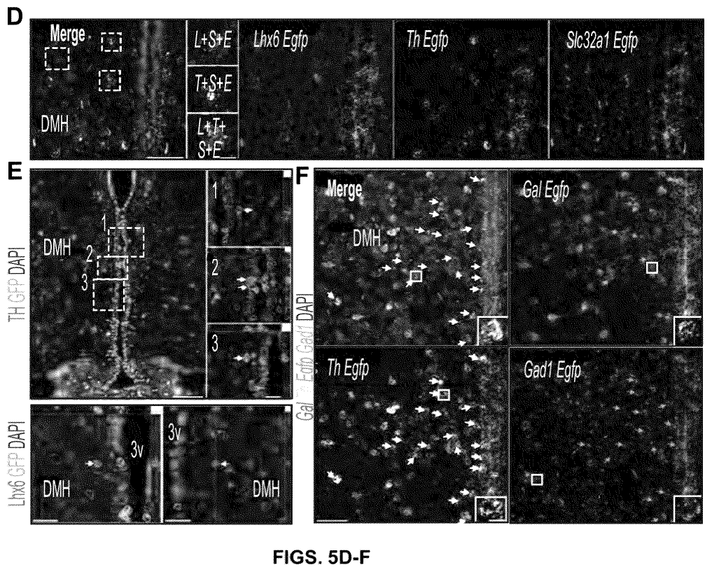

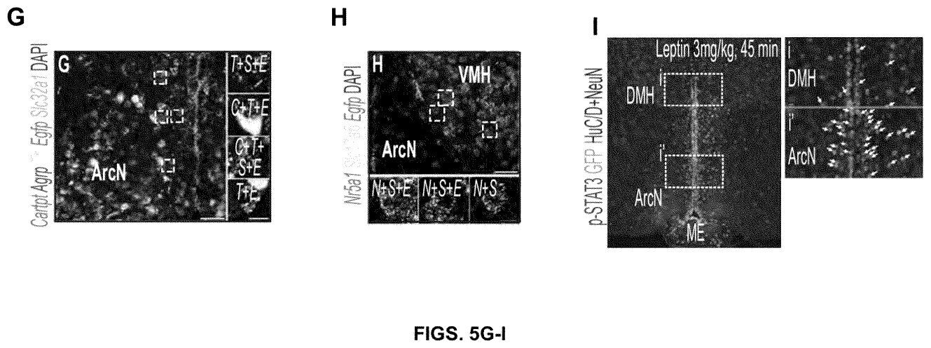

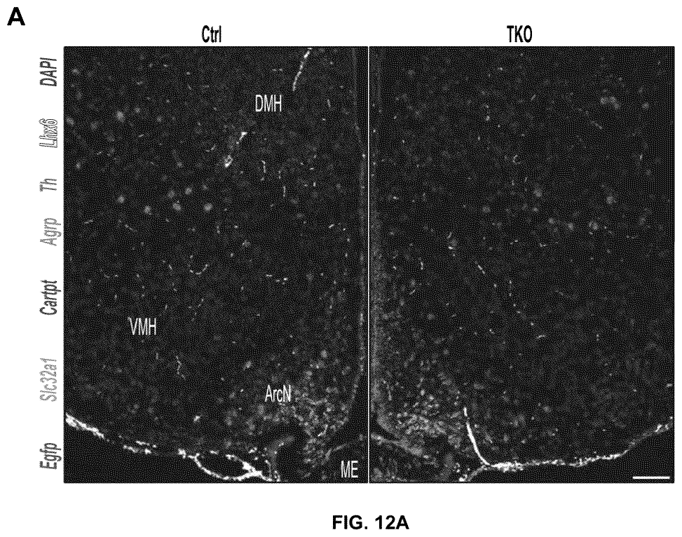

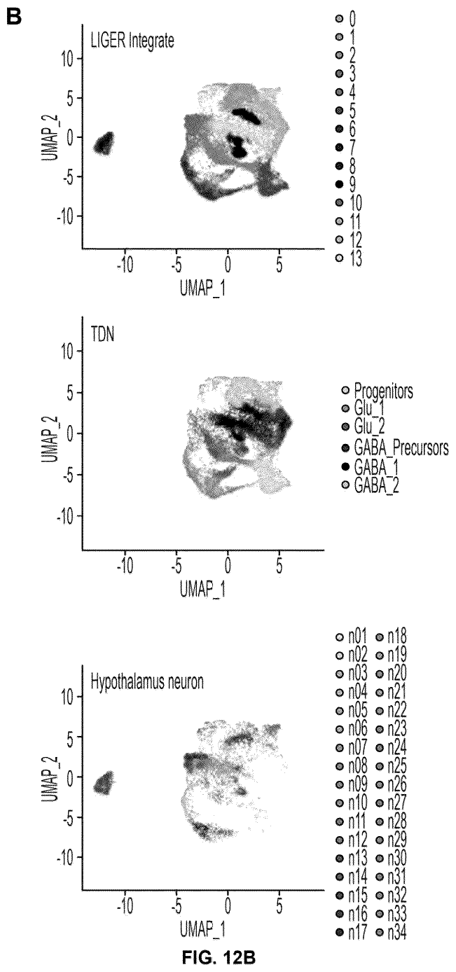

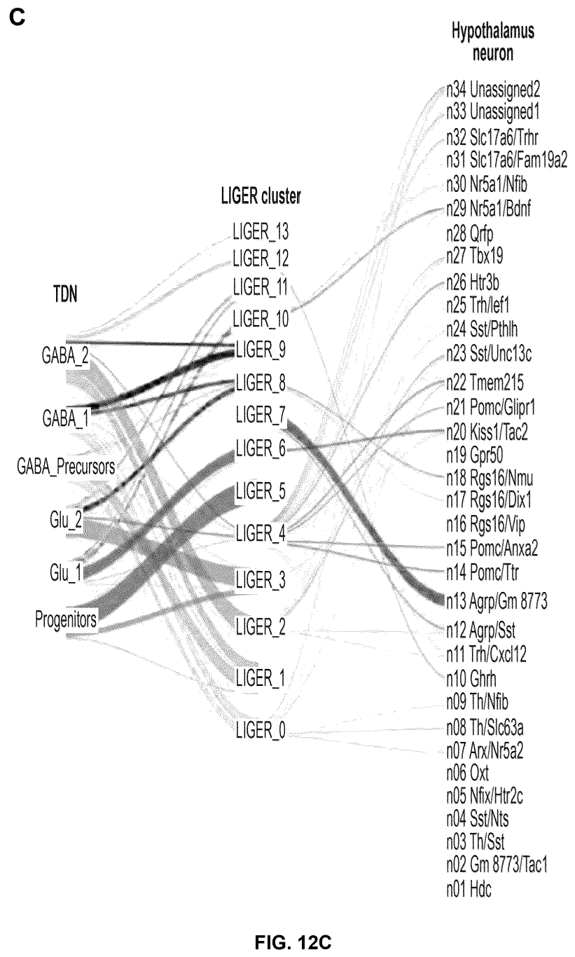

Tanycyte-derived neurons are predominantly found in the DMH and ArcN, with much smaller numbers detected in the VMH and ME (FIG. 1H). They are mostly GABAergic, and express molecular markers of DMH and ArcN neurons (FIGS. 5A and 5B), and substantial subsets closely match scRNA-Seq profiles of neuronal subtypes obtained from ArcN and VMH (FIGS. 12A-12C) (23). They include neuronal subtypes that regulate feeding, sleep, and directly regulate pituitary function, and many other subtypes whose function has yet to be characterized. In light of findings that tanycyte-derived neurogenesis can be stimulated by dietary and hormonal signals, and potentially modulate body weight and activity levels (3, 7), this raises the possibility that different internal states may trigger generation and/or survival of functionally distinct tanycyte-derived neuronal subtypes, leading to long-term changes in hypothalamic neural circuitry and physiological function.

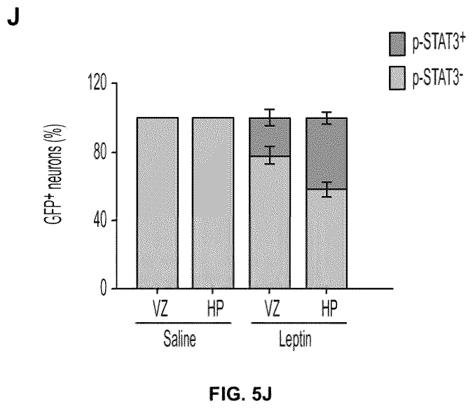

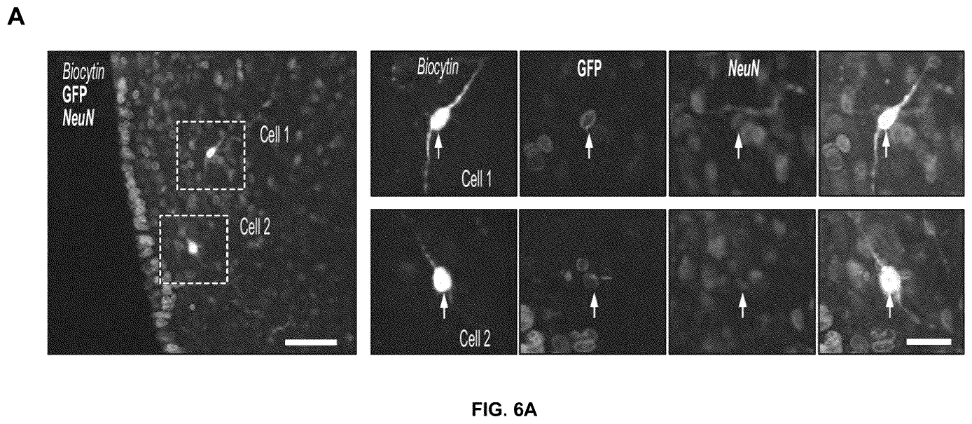

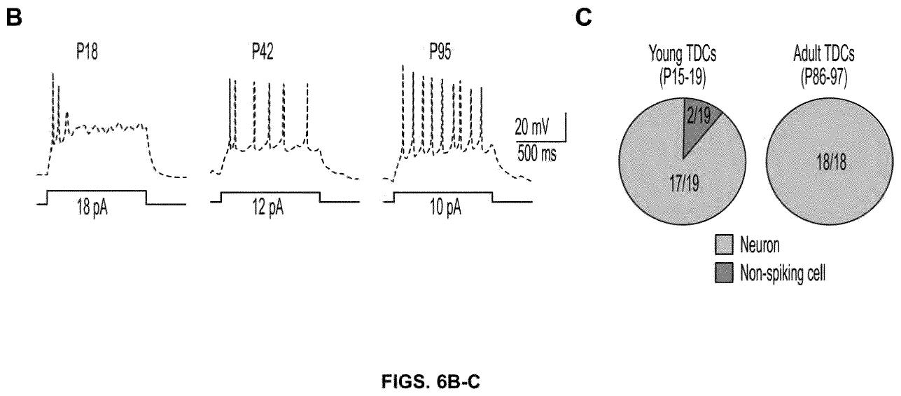

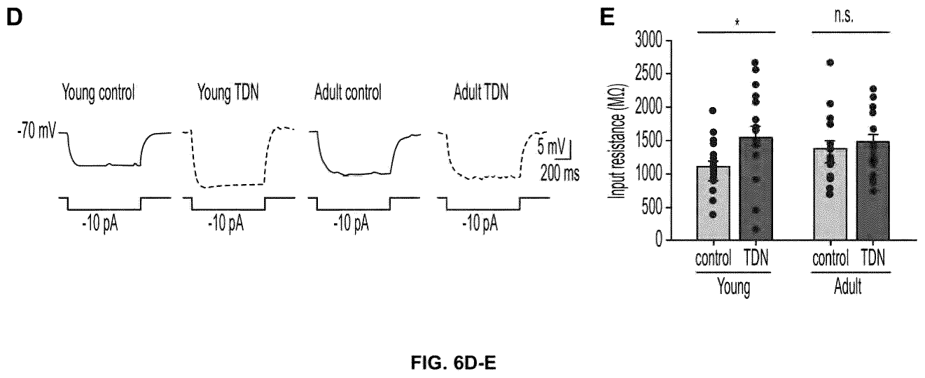

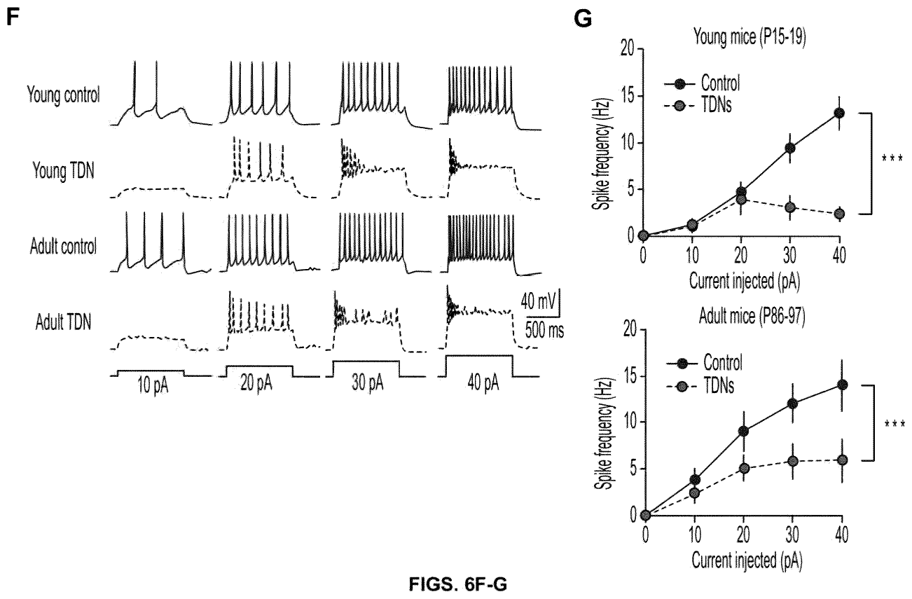

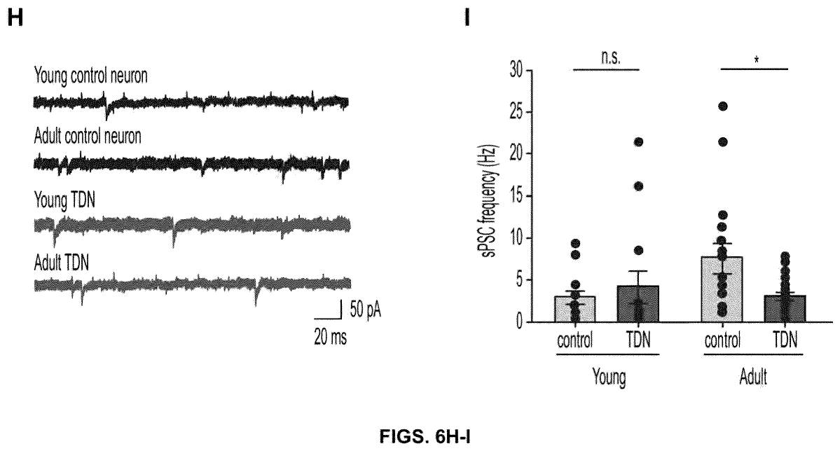

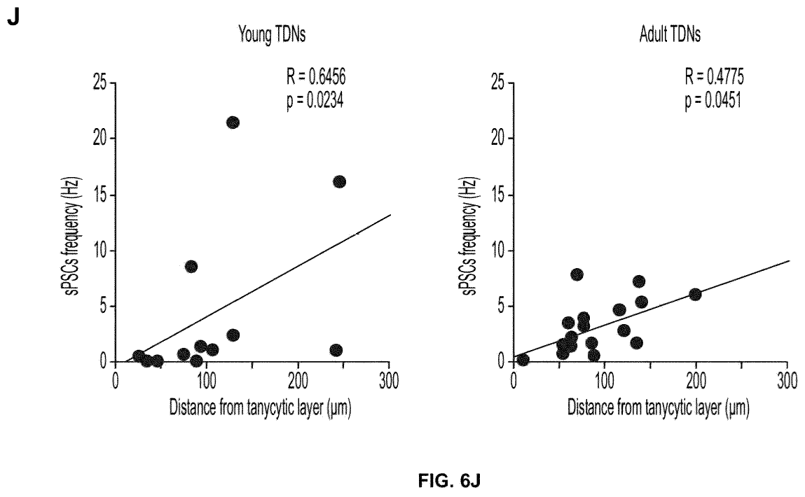

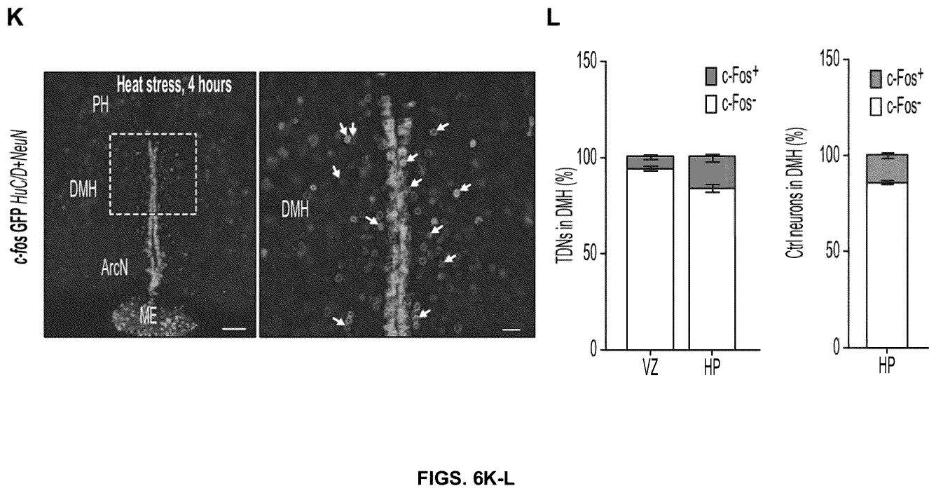

Tanycyte-derived neurons survived for months, integrate into hypothalamic circuitry, with subsets showing c-fos induction in response to heat stress (FIGS. 6A-6L). Older tanycyte-derived neurons show more sPSCs than younger tanycyte-derived neurons, and their input resistance lowers to become equivalent to that of GFP-negative nearby neurons, demonstrating progressive maturation. However, the number of both recorded sPSCs and the spike frequency of tanycyte-derived neurons remains consistently lower than those of GFP-negative neurons. This may be an intrinsic property of tanycyte-derived neurons. Alternatively, the excess tanycyte-derived neurons generated from Nfia/b/x-deficient tanycytes may form synaptic connections less efficiently. Distinguishing these possibilities will require electrophysiological recording of tanycyte-derived neurons from control animals, although the far smaller population of these cells in wildtype animals make this experiment very challenging.

Levels of tanycyte-derived neurogenesis are low under baseline conditions, and these studies both identify molecular mechanisms that might be modulated in response to changing physiological states, and identify means by which tanycyte-derived neurogenesis might be induced in wildtype animals. These findings also identify new means of enhancing neurogenic competence in mature tanycytes, potentially helping to guide the differentiation of specific subtypes of tanycyte-derived neurons, allowing for more selective long-term modulation of behavioral states.

### Nuclear Factor 1

Nuclear factor I (NFI) proteins constitute a family of dimeric DNA-binding proteins with similar, and possibly identical, DNA-binding specificity. They function as cellular transcription factors and as replication factors for adenovirus DNA replication. Diversity in this protein family is generated by multiple genes, differential splicing, and heterodimerization.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with a hypothalamic-regulated behavior and compared to samples from known conditions, e.g., a subject (or subjects) that does not have a hypothalamic-regulated behavior (a negative or normal control), or a subject (or subjects) who does have a hypothalamic-regulated behavior (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have a hypothalamic-regulated behavior in a healthy or general population. The amount of a compound can be measured in a test sample and compared to the “normal control” level, utilizing techniques such as reference limits, discrimination limits, or risk defining thresholds to define cutoff points and abnormal values (e.g., for a hypothalamic-regulated behavior or a symptom thereof). The normal control level means the level of one or more compounds or combined compounds typically found in a subject known not suffering from a hypothalamic-regulated behavior. Such normal control levels and cutoff points may vary based on whether a compounds is used alone or in a formula combining with other compounds into an index. Alternatively, the normal control level can be a database of compounds patterns from previously tested subjects who did not develop a hypothalamic-regulated behavior or a particular symptom thereof (e.g., in the event the a hypothalamic-regulated behavior develops or a subject already having a hypothalamic-regulated behavior is tested) over a clinically relevant time horizon.

The level that is determined may be the same as a control level or a cut off level or a threshold level or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,−2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterelogous nucleic acid sequence.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-a-methyl amino acids, C-a-methyl amino acids, b-methyl amino acids and D- or L-b-amino acids. Other non-naturally occurring amino acids include, for example, b-alanine (b-Ala), norleucine (Nle), norvaline (Nva), homoarginine (Har), 4-aminobutyric acid (g-Abu), 2-aminoisobutyric acid (Aib), 6-aminohexanoic acid (e-Ahx), ornithine (orn), sarcosine, a-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### Pharmaceutical Compositions and Formulations

The present invention provides pharmaceutical compositions comprising an effective amount of a composition (e.g., a composition comprising the agent that decreases the activity or expression of a nuclear factor I (NFI) gene or transcription factor) and at least one pharmaceutically acceptable excipient or carrier, wherein the effective amount is as described above in connection with the methods of the invention.

In one embodiment, the composition (e.g., a composition comprising the agent that decreases the activity or expression of a nuclear factor I (NFI) gene or transcription factor) is further combined with at least one additional therapeutic agent in a single dosage form. In one embodiment, the at least one additional therapeutic agent comprises taxanes.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

Kits Comprising the Agent that Decreases the Activity or Expression of a Nuclear Factor I (NFI) Gene or Transcription Factor

In embodiments, the kit comprises the agent that decreases the activity or expression of a nuclear factor I (NFI) gene or transcription factor and reagents.

In embodiments, the agent that decreases the activity or expression of a nuclear factor I (NFI) gene or transcription factor in the kit is suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a hypothalamic-regulated behavior), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Nfia/b/x Loss of Function Induces Proliferation and Neurogenesis in Neonatal Mice

To determine whether hypothalamic tanycytes express Nfia/b/x, bulk RNA-Seq data obtained from FACS-isolated GFP-positive cells from the mediobasal hypothalamus of adult Rax-GFP transgenic mice were analyzed, where GFP is selectively expressed in tanycytes (18, 19). GFP-positive tanycytes, indicated by Rax expression, show highly enriched expression for Nfia, Nfib, and Nfix relative to the GFP-negative, neuronally-enriched fraction of mediobasal hypothalamic cells (FIG. 1A). Immunohistochemical analysis in adult mice reveals that Nfia/b/x proteins are highly expressed in the tanycytic layer and in other hypothalamic glial cells in adult mice (FIG. 1B) (17). Rax-CreER; Nfia^{lox/lox}; Nfib^{lox/lox}; Nfix^{lox/lox}; CAG-lsl-Sun1-GFP mice (TKO mice hereafter) were generated, which allow inducible, tanycyte-specific disruption of Nfia/b/x function while simultaneously tracking the fate of tanycyte-derived cells using Cre-dependent Sun1-GFP expression (FIG. 1C) (14, 18, 20).

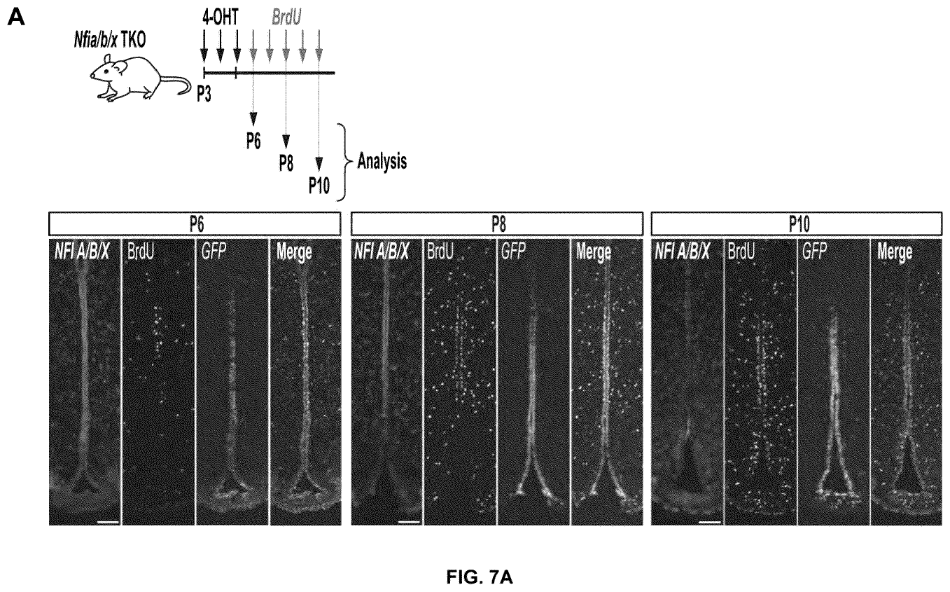

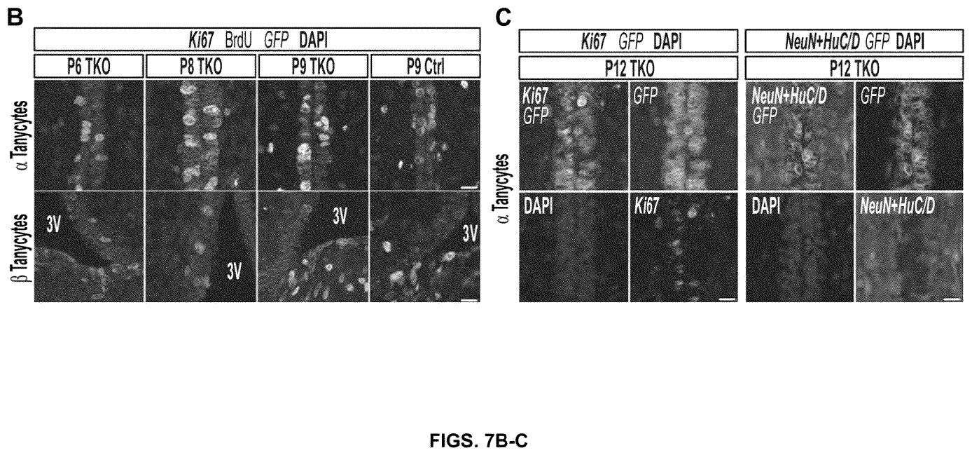

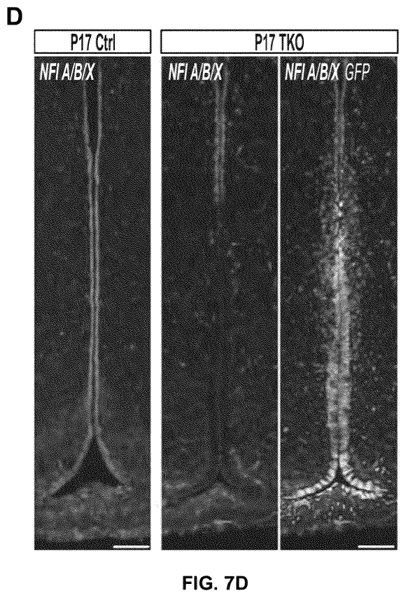

Cre activity was induced using daily intraperitoneal (i.p.) injections of 4-hydroxytamoxifen (4-OHT) between postnatal day 3 and day 5 (P3 and P5) (FIG. 1D). At this point, neurogenesis in the mediobasal hypothalamus is low under baseline conditions (3, 9). Following 4-OHT treatment, NFIA/B/X immunoreactivity was first reduced in the tanycyte layer beginning at P6 following 4-OHT injections between P3 and P5, initially in more ventral regions where Rax expression is strongest (FIG. 7A). NFIA/B/X immunoreactivity is largely undetectable by P10, and Cre-dependent GFP expression was correspondingly induced (FIG. 7A). BrdU incorporation and Ki67 labeling was seen beginning at P6 in dorsally-located alpha tanycytes, with labeling spreading to beta1 tanycytes of the arcuate nucleus by P8, and beta2 tanycytes of the median eminence by P10 (FIGS. 7A and 7B). At P12, Ki67 labeling was observed in the tanycyte layer immediately adjacent to the third ventricle lumen, and a small subset of GFP-positive cells in the tanycyte layer closest to the hypothalamic parenchyma began to express neuronal markers (FIG. 7C).

By P17, Nfia/b/x expression was completely lost in the tanycytes, although was preserved in Rax-negative ependymal cells where GFP expression is not induced (FIG. 7D), and tanycyte-derived GFP-positive neuronal precursors in the ventricular zone were actively amplified and had begun to migrate outward into the hypothalamic parenchyma in Nfia/b/x-deficient mice (FIG. 1E, 1F, FIG. 7D). In contrast, few migrating GFP-positive cells were observed in Rax-CreER; CAG-lsl-Sun1-GFP controls (FIG. 1E, FIG. 1F). At P45, a substantial increase in GFP-positive cells expressing mature neuronal markers was observed in the parenchyma of the arcuate (ArcN) and dorsomedial (DMH) hypothalamic nuclei (FIG. 1G, FIG. 1H), while limited numbers of GFP-positive cells expressing neuronal markers remained in the subventricular region in Nfia/b/x-deficient mice (FIG. 1G, yellow arrowheads, and FIG. 1I).

### Example 2: Loss of Function of Nfia/b/x Induces Tanycyte Proliferation in Older Mice

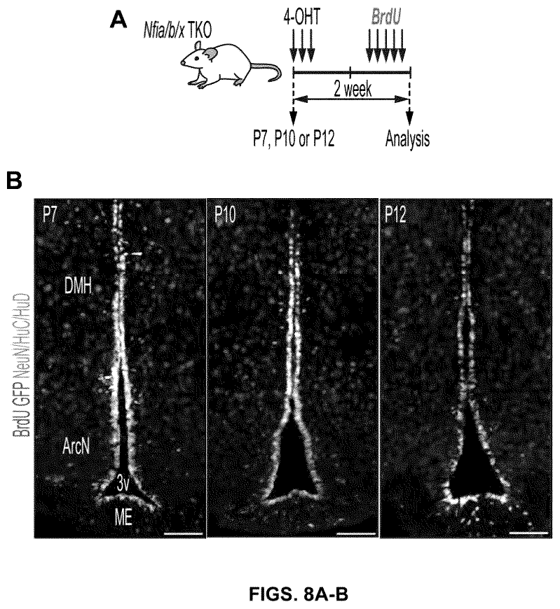

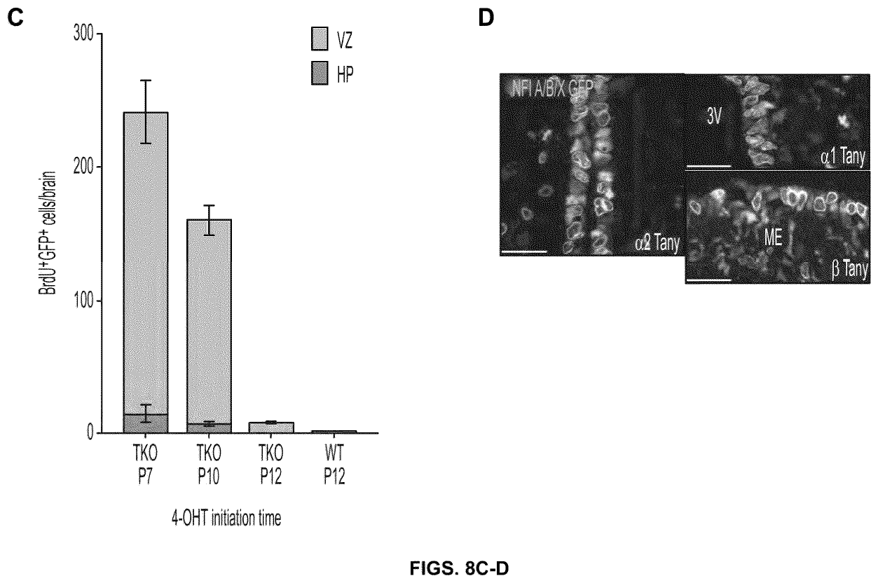

Studies have shown that loss of Nfia/b/x in late-stage retinal progenitors robustly induces proliferation and neurogenesis under baseline conditions, while in adult Müller glia, it induces limited levels of proliferation and neurogenesis, but only following neuronal injury (14, 15). This suggests that neurogenic competence in mature murine tanycytes could be lower than that seen in neonates. To test this, 4-OHT treatment was tested at older ages. While a robust induction of proliferation and neurogenesis following treatment at P7 was observed, this was less effective at P10. Only very low levels were observed at P12, not significantly different from control mice (FIGS. 8A, 8B and 8C). However, this low level of proliferation reflected a substantially reduced efficiency of 4-OHT-dependent disruption of Nfia/b/x as confirmed by the largely intact pattern of immunoreactivity for Nfia/b/x in TKO mice (FIG. 8D).

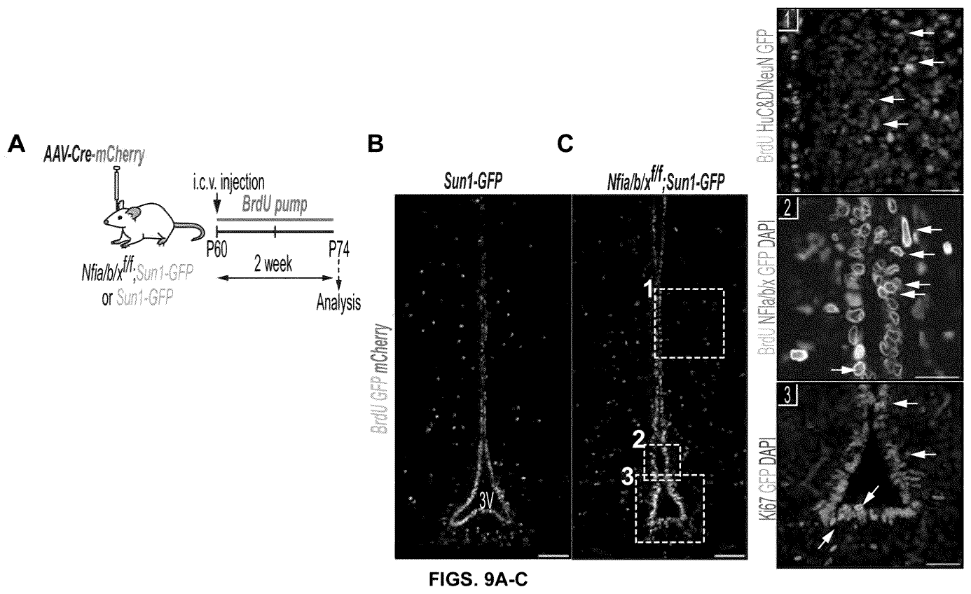

To improve the efficiency of Nfia/b/x deletion, and to study the effects of NFI loss of function in adult animals, viral-mediated Cre delivery was applied. Intracerebroventricular (i.e.v.) injection of AAV1-Cre-mCherry into both Nfia^{lox/lox}; Nfib^{lox/lox}; Nfix^{lox/lox}; CAG-lsl-Sun1-GFP and CAG-lsl-Sun1-GFP control mice at P60 resulted in robust mCherry expression in ventricular hypothalamic cells within two weeks, along with Cre-dependent induction of Sun1-GFP expression in both control and Nfia/b/x floxed conditional mice (FIGS. 9A-9C). Efficient loss of NFI expression in GFP-positive tanycytes by P74 and co-immunolabeling was observed with BrdU, delivered continuously by osmotic mini-pump during the two weeks (FIG. 9C). To determine the specific induction of proliferation initiated from tanycytes, EdU was administered using a once daily i.p. injection between P76-P78, and analyzed mice at P79. Selective EdU incorporation into alpha tanycytes adjacent to the dorsal part of ArcN (FIGS. 1L, 1, M) was observed. Much lower levels of EdU incorporation were observed in ventrally located beta tanycytes, while no EdU labeling was observed in controls (FIG. 1K). Although a few GFP-positive cells in the mediobasal hypothalamic parenchyma were observed, these cells were not labeled with EdU, and there was no difference in their number between control and TKO animals (FIG. 1N). The specific and local induction of tanycyte proliferation with the increased endogenous expression of Ki67, a proliferation marker, only in Nfia/b/x-deficient TKO mice (FIGS. 1O, FIG. 9C) was observed.

### Example 3: Single Cell RNA-Seq and ATAC-Seq Analysis Identified Gene Regulatory Networks Controlling Neurogenesis in Tanycytes

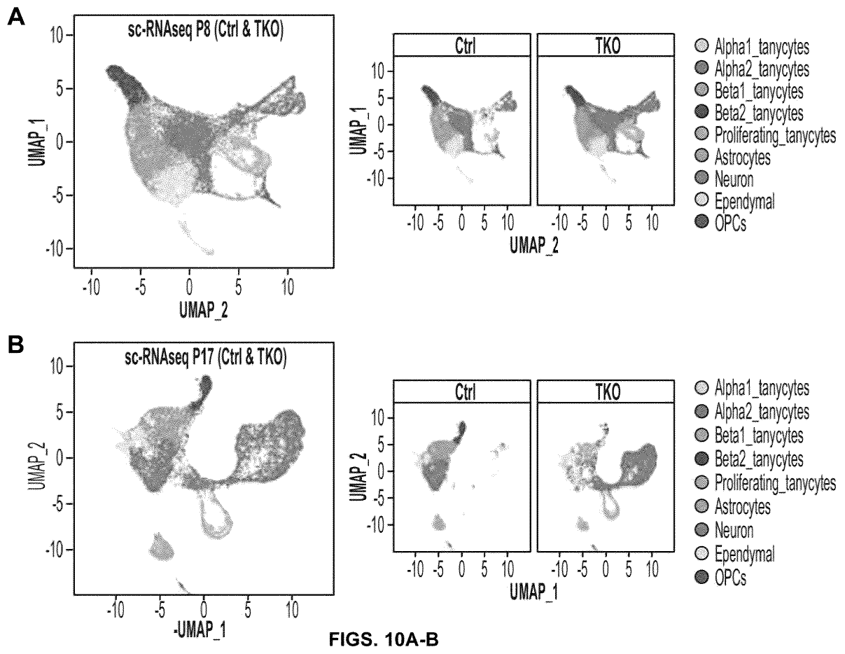

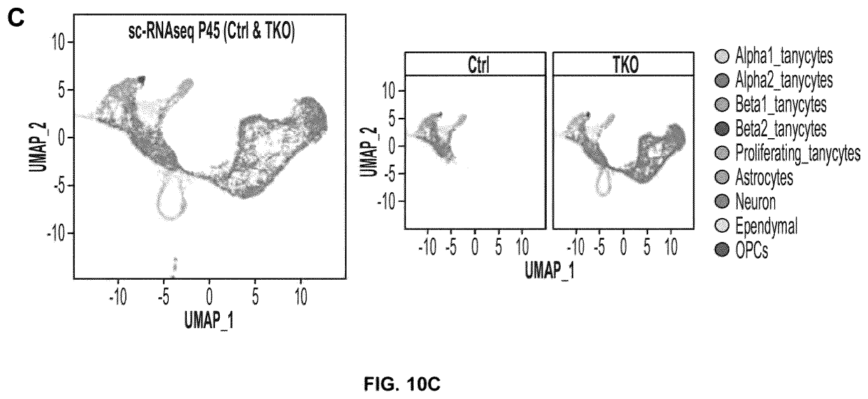

To obtain a comprehensive picture of the molecular mechanisms that regulate tanycyte proliferation and neurogenic competence, scRNA-Seq analysis was conducted of FACS-isolated GFP-positive tanycytes and tanycyte-derived cells from both control and Nfia/b/x-deficient mice. To do this, Cre activity was induced in tanycytes between P3 and P5 and harvested GFP-positive cells at P8, P17 and P45 timepoints in TKO mice. A total of >60,000 cells were profiled using the Chromium platform (10×Genomics), generated separate UMAP plots for controls, Nfia/b/x-deficient tanycytes, and then aggregated data obtained from all samples (FIG. 2A, and FIG. 10A-10C). In control mice, tanycyte subtypes (alpha1, alpha2, beta1, beta2) were readily distinguished, based on previously characterized molecular markers (21-23), but also observed that tanycytes give rise to a range of other hypothalamic cell types (FIG. 2B). In controls, at P8, a small fraction of proliferative tanycytes was observed, from which arise differentiation trajectories that give rise to astrocytes and ependymal cells, as well as small numbers of oligodendrocyte progenitor cells and neurons (FIG. 2C, 2D, FIGS. 10A-10C, Table 2, below).

TABLE 2
Number of each cell type identified in each scRNA-Seq library

| | | | | | | | | | | |
|---|---|---|---|---|---|---|---|---|---|---|
| | | | | | Prolif- | | Astro- | Epen- | | |
| Samples | Alpha1_tancytes | Alpha2_tancytes | Beta1_tancytes | Beta2_tancytes | erating_tancytes | Neuron | cytes | dymal | OPCs | Total |

Number of cells that belong to each celltype at each condition

| | | | | | | | | | | |
|---|---|---|---|---|---|---|---|---|---|---|
| P8_Ctrl | 875 | 2143 | 1906 | 583 | 129 | 526 | 252 | 74 | 121 | 6609 |
| P8_TKO | 1147 | 3347 | 3276 | 1006 | 1530 | 1764 | 261 | 372 | 227 | 12930 |
| P17_Ctrl | 657 | 2011 | 2163 | 885 | 8 | 44 | 631 | 20 | 75 | 6494 |
| P17_TKO | 343 | 1648 | 1495 | 382 | 1199 | 6947 | 274 | 31 | 94 | 12413 |
| P45_Ctrl | 443 | 780 | 755 | 185 | 4 | 12 | 302 | 63 | 63 | 2607 |
| P45_TKO | 474 | 3043 | 1429 | 334 | 929 | 6778 | 315 | 77 | 38 | 13417 |

Percentage (%) of cells that belong to each celltype at each condition

| | | | | | | | | | | |
|---|---|---|---|---|---|---|---|---|---|---|
| P8_Ctrl | 13.2 | 32.4 | 28.8 | 8.8 | 2.0 | 8.0 | 3.8 | 1.1 | 1.8 | 100 |
| P8_TKO | 8.9 | 25.9 | 25.3 | 7.8 | 11.8 | 13.6 | 2.0 | 2.9 | 1.8 | 100 |
| P17_Ctrl | 10.1 | 31.0 | 33.3 | 13.6 | 0.1 | 0.7 | 9.7 | 0.3 | 1.2 | 100 |
| P17_TKO | 2.8 | 13.3 | 12.0 | 3.1 | 9.7 | 56.0 | 2.2 | 0.2 | 0.8 | 100 |
| P45_Ctrl | 17.0 | 29.9 | 29.0 | 7.1 | 0.2 | 0.5 | 11.6 | 2.4 | 2.4 | 100 |
| P45_TKO | 3.5 | 22.7 | 10.7 | 2.5 | 6.9 | 50.5 | 2.3 | 0.6 | 0.3 | 100 |

At P17 and P45, however, very few proliferating tanycytes are observed, and evidence for ongoing generation of neurons and glia is lacking (FIG. 2C and FIG. 2D, FIG. 10A-10C). In Nfia/b/x-deficient tanycytes, in contrast, a significantly larger fraction of cells were proliferating tanycytes at all ages, along with clear evidence for ongoing neurogenesis (FIG. 2C, FIGS. 10A-10C). Furthermore, a substantial reduction in the relative fraction of non-neuronal cells—including astrocytes, oligodendrocyte progenitor cells, and ependymal cells—is observed, in line with previous studies reporting an essential role for NFI family genes in gliogenesis in other CNS regions (14, 24-26). While controls show a higher fraction of tanycyte-derived astrocytes, a much higher fraction of GFP-positive cells are neurons in TKO mice (FIG. 2D, Table 2, above). Furthermore, the density and relative fraction of cells expressing alpha2 tanycyte markers is increased in Nfia/b/x-deficient mice, which is consistent with previously published neurosphere and cell lineage analysis (4), demonstrating a higher neurogenic competence for alpha2 tanycytes (FIG. 2C bottom, FIG. 2D, Table 2, above) (4).

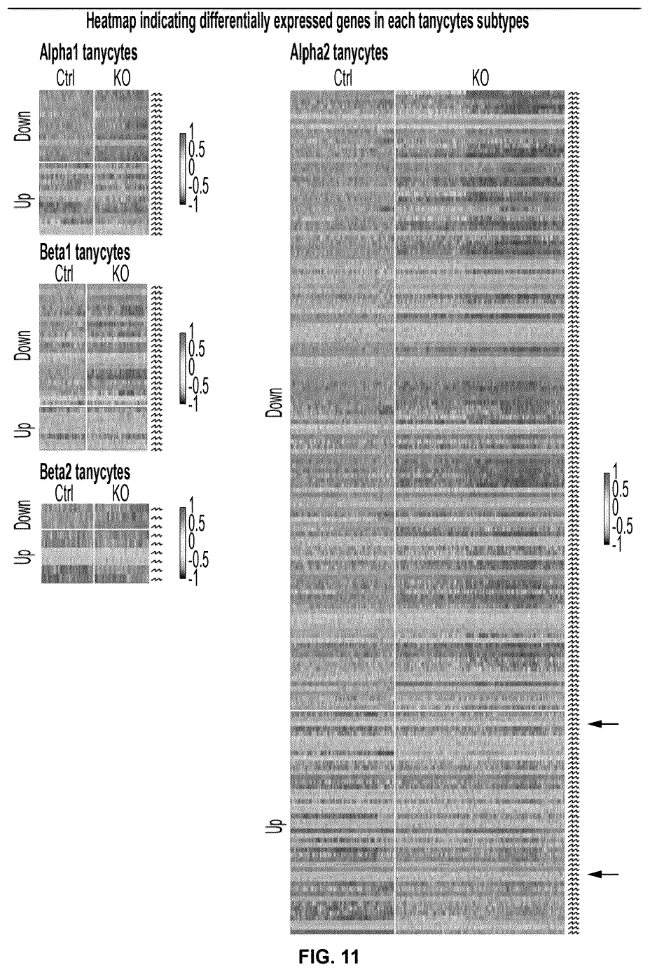

To identify critical regulators of proliferative and neurogenic competence in tanycytes, a differential gene expression analysis was performed between control and Nfia/b/x-deficient mice in each of tanycyte subtypes. This analysis uncovered differential expression of multiple extrinsic and intrinsic regulators of these processes, particularly in alpha2 tanycytes (FIG. 11, Table 4, FIG. 2E).

TABLE 4
Differentially expressed genes in Nfia/b/x-deficient alpha2 tanycytes
ident. 1 = ‘TKO’, ident. 2 = ‘Ctrl’, min. pct = 0.05, logfc. threshold = 0.15

| genes | p_val | avg_logFC | pct. 1 | pct. 2 | p_val_adj |
|---|---|---|---|---|---|
| Meg3 | 0 | 0.556991148 | 0.989 | 1 | 0 |
| Shh | 0 | 0.284157888 | 0.375 | 0.994 | 0 |
| Rbp4 | 0 | −0.196010745 | 0.336 | 0.287 | 0 |
| Apoe | 0.00E+00 | −0.471939901 | 0.995 | 0.999 | 0.00E+00 |
| Lmo2 | 1.88E−260 | 0.194923972 | 0.474 | 0.99 | 2.98E−257 |
| Dlk1 | 8.99E−259 | 1.297116338 | 0.726 | 0.999 | 1.42E−255 |
| Ramp1 | 1.66E−233 | −0.407515285 | 0.77 | 0.847 | 2.63E−230 |
| Dbi | 2.44E−232 | −0.303978792 | 0.998 | 0.997 | 3.87E−229 |
| S100a6 | 1.57E−220 | −0.277451959 | 0.981 | 0.996 | 2.48E−217 |
| Cst3 | 7.22E−211 | −0.382893525 | 1 | 1 | 1.14E−207 |
| Mt3 | 3.48E−189 | −0.265464204 | 0.98 | 0.995 | 5.51E−186 |
| S100a1 | 7.93E−188 | −0.27534631 | 0.698 | 0.876 | 1.25E−184 |
| Bcan | 7.90E−187 | −0.299965322 | 0.807 | 0.872 | 1.25E−183 |
| Ndrg2 | 6.91E−184 | −0.252086518 | 0.85 | 0.92 | 1.09E−180 |
| Hspa2 | 1.87E−182 | −0.281108776 | 0.827 | 0.892 | 2.96E−179 |
| Ecel1 | 1.70E−177 | −0.228855 | 0.263 | 0.336 | 2.69E−174 |
| Ntrk2 | 6.82E−162 | −0.196647382 | 0.998 | 0.999 | 1.08E−158 |
| Eef1a1 | 8.72E−159 | 0.180562641 | 0.999 | 1 | 1.38E−155 |
| Syt4 | 9.78E−156 | 0.269693783 | 0.371 | 0.923 | 1.55E−152 |
| Clu | 1.66E−154 | −0.178702551 | 0.411 | 0.372 | 2.62E−151 |
| Col23a1 | 1.24E−149 | −0.246615685 | 0.844 | 0.894 | 1.97E−146 |
| Sparcl1 | 4.24E−149 | −0.376132895 | 0.625 | 0.896 | 6.71E−146 |
| Nnat | 3.14E−143 | −0.157976537 | 1 | 1 | 4.98E−140 |
| Fmo1 | 1.88E−139 | −0.159437296 | 0.578 | 0.423 | 2.98E−136 |
| Dnah12 | 1.20E−136 | −0.221644858 | 0.612 | 0.811 | 1.89E−133 |
| Gria1 | 4.98E−134 | 0.255435479 | 0.414 | 0.928 | 7.89E−131 |
| Stmn1 | 1.20E−132 | 0.374090974 | 0.943 | 1 | 1.91E−129 |
| 6330403K07Rik | 5.33E−129 | −0.177125894 | 0.938 | 0.988 | 8.43E−126 |
| Nr2f2 | 1.65E−123 | 0.200063002 | 0.47 | 0.9 | 2.61E−120 |
| Fst | 4.12E−122 | 0.561992832 | 0.437 | 0.994 | 6.52E−119 |
| Sox4 | 4.09E−121 | 0.42599001 | 0.893 | 0.994 | 6.48E−118 |
| Tubb4b | 1.00E−119 | −0.232290587 | 0.963 | 0.98 | 1.59E−116 |
| Gas1 | 1.08E−119 | −0.225259611 | 0.547 | 0.441 | 1.71E−116 |
| Fos | 4.52E−117 | −0.273760371 | 0.991 | 0.997 | 7.16E−114 |
| Dynlrb2 | 2.62E−115 | −0.26415725 | 0.778 | 0.839 | 4.15E−112 |
| Ccdc80 | 2.68E−114 | −0.175206889 | 0.555 | 0.424 | 4.25E−111 |
| Mest | 9.37E−110 | 0.375062011 | 0.885 | 0.983 | 1.48E−106 |
| Fbxo2 | 7.09E−103 | −0.190455612 | 0.675 | 0.788 | 1.12E−99 |
| Cavin3 | 3.68E−102 | −0.22758988 | 0.784 | 0.813 | 5.82E−99 |
| Prdx6 | 3.47E−98 | −0.165945462 | 0.98 | 0.993 | 5.49E−95 |
| Crym | 7.49E−97 | −0.387571503 | 0.921 | 0.985 | 1.19E−93 |
| Tubb5 | 8.14E−97 | 0.311256292 | 0.963 | 0.998 | 1.29E−93 |
| Cd63 | 8.99E−96 | −0.154263389 | 0.987 | 0.993 | 1.42E−92 |
| Cfap126 | 2.80E−95 | −0.239397473 | 0.907 | 0.984 | 4.43E−92 |
| Mllt11 | 6.83E−95 | 0.175683458 | 0.418 | 0.82 | 1.08E−91 |
| Fabp5 | 8.75E−95 | −0.301792246 | 0.887 | 0.944 | 1.38E−91 |
| Tgfb2 | 1.33E−94 | −0.217090472 | 0.456 | 0.41 | 2.11E−91 |
| Mgst1 | 1.88E−92 | −0.1895591 | 0.926 | 0.969 | 2.98E−89 |
| Hepacam | 8.42E−90 | −0.233286178 | 0.838 | 0.878 | 1.33E−86 |
| Islr | 2.05E−89 | −0.246676252 | 0.693 | 0.791 | 3.24E−86 |
| 1700007K13Rik | 2.59E−88 | −0.185980719 | 0.656 | 0.872 | 4.11E−85 |
| Lrp1b | 1.55E−85 | 0.237124665 | 0.528 | 0.973 | 2.46E−82 |
| Thrsp | 3.04E−85 | −0.202661475 | 0.78 | 0.859 | 4.82E−82 |
| Gja1 | 4.03E−85 | −0.28186088 | 0.782 | 0.819 | 6.38E−82 |
| Fxyd1 | 7.13E−85 | −0.196014592 | 0.625 | 0.788 | 1.13E−81 |
| Mt1 | 1.78E−84 | −0.150978234 | 0.943 | 0.99 | 2.81E−81 |
| Enho | 2.93E−84 | −0.215787837 | 0.741 | 0.801 | 4.64E−81 |
| Id4 | 9.96E−84 | −0.153932683 | 0.915 | 0.96 | 1.58E−80 |
| Flt1 | 1.02E−83 | −0.23966225 | 0.608 | 0.449 | 1.62E−80 |
| Hnrnpa1 | 2.77E−83 | 0.261572038 | 0.908 | 0.982 | 4.39E−80 |
| Kcnj10 | 5.68E−83 | −0.15169769 | 0.551 | 0.445 | 8.99E−80 |
| Timp4 | 2.15E−78 | −0.258653778 | 0.685 | 0.853 | 3.41E−75 |
| Fam183b | 7.29E−77 | −0.174602492 | 0.888 | 0.966 | 1.15E−73 |
| Rpl29 | 9.65E−77 | 0.242361665 | 0.919 | 1 | 1.53E−73 |
| Gstm1 | 1.17E−74 | −0.194658321 | 0.737 | 0.854 | 1.86E−71 |
| Prss56 | 3.46E−74 | −0.316988669 | 0.429 | 0.446 | 5.48E−71 |
| Adamts1 | 7.17E−74 | −0.175202535 | 0.573 | 0.495 | 1.13E−70 |
| 1700016K19Rik | 1.21E−71 | −0.209864339 | 0.714 | 0.864 | 1.92E−68 |
| Tsc22d4 | 1.94E−71 | −0.174719778 | 0.807 | 0.902 | 3.07E−68 |
| Prss23 | 1.49E−69 | 0.361252242 | 0.51 | 0.993 | 2.35E−66 |
| Tagln2 | 3.50E−69 | −0.20799568 | 0.739 | 0.817 | 5.54E−66 |
| Jun | 1.76E−68 | −0.304604383 | 0.92 | 0.979 | 2.78E−65 |
| Elof1 | 2.67E−67 | −0.163728055 | 0.845 | 0.927 | 4.23E−64 |
| Csrp1 | 4.24E−65 | −0.164238041 | 0.625 | 0.744 | 6.72E−62 |
| Klf2 | 1.04E−61 | −0.39479271 | 0.758 | 0.93 | 1.64E−58 |
| 2410004P03Rik | 6.03E−60 | −0.188485863 | 0.811 | 0.814 | 9.54E−57 |
| Prelp | 1.26E−59 | −0.151883119 | 0.539 | 0.47 | 2.00E−56 |
| Hes1 | 3.57E−59 | −0.255532571 | 0.86 | 0.929 | 5.65E−56 |
| Cmtm5 | 3.61E−58 | −0.233292574 | 0.576 | 0.454 | 5.71E−55 |
| Pifo | 7.21E−58 | −0.170169509 | 0.884 | 0.969 | 1.14E−54 |
| Gclm | 2.79E−57 | −0.208730648 | 0.757 | 0.856 | 4.41E−54 |
| Anxa2 | 5.52E−57 | −0.17338122 | 0.849 | 0.932 | 8.73E−54 |
| Egr1 | 1.15E−56 | −0.179866305 | 0.678 | 0.845 | 1.83E−53 |
| Marcks | 3.33E−56 | 0.163339056 | 0.95 | 0.998 | 5.28E−53 |
| Igfbp5 | 4.46E−56 | 0.213647926 | 0.836 | 0.988 | 7.06E−53 |
| Aldoc | 5.47E−56 | −0.309944426 | 0.561 | 0.655 | 8.67E−53 |
| Bcl11a | 5.82E−56 | 0.216875948 | 0.385 | 0.777 | 9.21E−53 |
| Dhrs7 | 1.69E−55 | −0.175853595 | 0.746 | 0.808 | 2.68E−52 |
| Sparc | 6.74E−55 | −0.151814012 | 0.993 | 0.998 | 1.07E−51 |
| Mns1 | 2.91E−52 | −0.19090517 | 0.71 | 0.818 | 4.60E−49 |
| Sfxn5 | 8.24E−51 | −0.188537091 | 0.752 | 0.785 | 1.30E−47 |
| Meig1 | 9.53E−51 | −0.17066218 | 0.847 | 0.91 | 1.51E−47 |
| S100a13 | 5.12E−49 | −0.152508641 | 0.626 | 0.753 | 8.11E−46 |
| Rarres2 | 2.96E−47 | −0.274981015 | 0.613 | 0.673 | 4.69E−44 |
| Xist | 7.34E−47 | 0.230231264 | 0.823 | 0.966 | 1.16E−43 |
| Slc25a5 | 9.18E−47 | 0.181178709 | 0.938 | 0.985 | 1.45E−43 |
| Serpinb1a | 9.72E−47 | −0.156117837 | 0.504 | 0.496 | 1.54E−43 |
| Dnah11 | 1.50E−46 | −0.152649591 | 0.58 | 0.524 | 2.37E−43 |
| Gsn | 6.50E−46 | −0.246570056 | 0.638 | 0.669 | 1.03E−42 |
| Atp1b2 | 1.60E−45 | −0.190287305 | 0.699 | 0.798 | 2.54E−42 |
| Plagl1 | 9.44E−45 | 0.261566046 | 0.832 | 0.995 | 1.49E−41 |
| Cfap43 | 4.61E−44 | −0.226063329 | 0.684 | 0.798 | 7.29E−41 |
| Jpt1 | 8.89E−44 | 0.187098478 | 0.836 | 0.954 | 1.41E−40 |
| Sat1 | 8.83E−43 | −0.2590837 | 0.693 | 0.779 | 1.40E−39 |
| Hes5 | 1.33E−42 | 0.668276893 | 0.527 | 0.931 | 2.10E−39 |
| Ubc | 1.95E−42 | −0.153513273 | 0.932 | 0.964 | 3.09E−39 |
| Lmo1 | 3.09E−41 | 0.285354327 | 0.527 | 0.63 | 4.90E−38 |
| Enkur | 5.51E−39 | −0.165265169 | 0.743 | 0.813 | 8.72E−36 |
| Myo6 | 2.02E−38 | −0.154893343 | 0.765 | 0.839 | 3.20E−35 |
| Cdkn1c | 2.91E−38 | 0.27345471 | 0.806 | 0.979 | 4.61E−35 |
| Srebf1 | 4.46E−37 | −0.203150306 | 0.655 | 0.779 | 7.06E−34 |
| Gm17455 | 4.59E−37 | −0.212485729 | 0.741 | 0.872 | 7.27E−34 |
| Ppp1r14b | 1.21E−36 | 0.19286602 | 0.87 | 0.972 | 1.91E−33 |
| Gm26735 | 1.41E−36 | 0.18976333 | 0.54 | 0.909 | 2.24E−33 |
| 3300002A11Rik | 3.53E−35 | −0.231341471 | 0.677 | 0.792 | 5.59E−32 |
| Junb | 1.65E−32 | −0.288013505 | 0.75 | 0.791 | 2.61E−29 |
| Ccnd2 | 9.53E−32 | 0.181461346 | 0.8 | 0.849 | 1.51E−28 |
| Timp3 | 1.25E−31 | −0.174437895 | 0.8 | 0.868 | 1.98E−28 |
| Clec3b | 1.91E−31 | −0.215844965 | 0.384 | 0.582 | 3.02E−28 |
| Col9a3 | 1.92E−29 | −0.267278492 | 0.675 | 0.729 | 3.05E−26 |
| Rmst | 8.58E−28 | −0.15539517 | 0.801 | 0.821 | 1.36E−24 |
| Klf6 | 2.09E−27 | −0.197384089 | 0.891 | 0.946 | 3.31E−24 |
| Dusp1 | 2.31E−27 | −0.288289053 | 0.689 | 0.808 | 3.66E−24 |
| Kcnq1ot1 | 9.37E−27 | 0.151836304 | 0.871 | 0.989 | 1.48E−23 |
| Spef2 | 8.44E−26 | −0.207888141 | 0.702 | 0.748 | 1.34E−22 |
| Igfbp2 | 1.47E−25 | 0.180841566 | 0.636 | 0.987 | 2.32E−22 |
| Hspa5 | 2.94E−25 | −0.195299393 | 0.922 | 0.985 | 4.66E−22 |
| 1700012B09Rik | 4.27E−25 | −0.193626556 | 0.656 | 0.77 | 6.76E−22 |
| Smad1 | 6.98E−24 | 0.301326782 | 0.787 | 0.973 | 1.10E−20 |
| Itih5 | 5.75E−23 | −0.188304109 | 0.625 | 0.586 | 9.10E−20 |
| Nrarp | 1.13E−21 | 0.180440897 | 0.409 | 0.715 | 1.80E−18 |
| Ier2 | 1.77E−20 | −0.199531522 | 0.782 | 0.853 | 2.79E−17 |
| Slc1a2 | 7.70E−20 | −0.285055371 | 0.643 | 0.644 | 1.22E−16 |
| S100a4 | 1.81E−19 | −0.359864053 | 0.635 | 0.64 | 2.87E−16 |
| Dbn1 | 2.04E−19 | 0.186136875 | 0.709 | 0.844 | 3.24E−16 |
| Phlda1 | 3.91E−19 | −0.160255022 | 0.55 | 0.578 | 6.19E−16 |
| Ephb1 | 9.66E−18 | 0.163113319 | 0.756 | 0.857 | 1.53E−14 |
| Cyr61 | 1.11E−17 | −0.316358898 | 0.819 | 0.827 | 1.76E−14 |
| Calml4 | 2.96E−17 | −0.210829558 | 0.647 | 0.748 | 4.68E−14 |
| Cfap100 | 7.19E−17 | −0.185106617 | 0.669 | 0.788 | 1.14E−13 |
| 1700007G11Rik | 7.46E−16 | −0.210797983 | 0.65 | 0.727 | 1.18E−12 |
| 1110017D15Rik | 8.48E−16 | −0.244676284 | 0.624 | 0.756 | 1.34E−12 |
| Gpld1 | 2.99E−14 | −0.163438191 | 0.576 | 0.573 | 4.74E−11 |
| Tub | 1.33E−13 | 0.192036626 | 0.459 | 0.766 | 2.10E−10 |
| Cadm2 | 2.03E−13 | −0.159017456 | 0.661 | 0.775 | 3.22E−10 |
| Zdbf2 | 2.99E−13 | 0.317576902 | 0.673 | 0.934 | 4.74E−10 |
| Zfp36 | 1.11E−12 | −0.238638914 | 0.616 | 0.59 | 1.76E−09 |
| Mfge8 | 1.38E−12 | −0.177491129 | 0.577 | 0.536 | 2.19E−09 |
| Bhlhe40 | 2.31E−12 | −0.153509155 | 0.611 | 0.752 | 3.66E−09 |
| Sulf1 | 3.56E−12 | 0.268686399 | 0.722 | 0.925 | 5.64E−09 |
| Acsl3 | 5.72E−12 | −0.195568684 | 0.625 | 0.69 | 9.05E−09 |
| Lrriq1 | 1.05E−11 | −0.155173786 | 0.732 | 0.752 | 1.66E−08 |
| Dcdc2a | 1.08E−11 | −0.208787436 | 0.696 | 0.701 | 1.71E−08 |
| Slc7a11 | 2.60E−11 | −0.296914826 | 0.505 | 0.521 | 4.12E−08 |
| Sfrp1 | 2.85E−11 | 0.187386915 | 0.814 | 0.97 | 4.52E−08 |
| Ptch1 | 5.03E−11 | 0.157291566 | 0.544 | 0.844 | 7.96E−08 |
| Dpysl3 | 3.50E−10 | 0.185841074 | 0.658 | 0.823 | 5.54E−07 |
| Notum | 3.57E−10 | 0.162949952 | 0.317 | 0.567 | 5.66E−07 |
| Gm26532 | 5.45E−09 | −0.174512921 | 0.626 | 0.667 | 8.62E−06 |
| Tnfrsf19 | 5.67E−09 | −0.18327157 | 0.537 | 0.574 | 8.97E−06 |
| Aqp4 | 5.98E−09 | −0.218608864 | 0.633 | 0.708 | 9.46E−06 |
| Ak7 | 7.76E−09 | −0.195968611 | 0.604 | 0.691 | 1.23E−05 |
| Pmepa1 | 1.15E−08 | 0.217542706 | 0.659 | 0.95 | 1.82E−05 |
| Dnah6 | 1.56E−08 | −0.172935765 | 0.626 | 0.718 | 2.47E−05 |
| Emp2 | 1.59E−08 | −0.23588835 | 0.582 | 0.527 | 2.51E−05 |
| Bambi | 4.94E−08 | −0.237234759 | 0.46 | 0.538 | 7.83E−05 |
| Slc2a1 | 9.25E−08 | −0.158162772 | 0.639 | 0.635 | 0.000146436 |
| Socs3 | 1.00E−07 | −0.27974381 | 0.696 | 0.759 | 0.000158678 |
| Foxj1 | 7.78E−07 | −0.182384613 | 0.634 | 0.701 | 0.001232115 |
| Ttc21a | 9.64E−07 | −0.209890659 | 0.656 | 0.681 | 0.001526732 |
| Cnp | 1.12E−06 | −0.170927661 | 0.625 | 0.657 | 0.001776208 |
| Ap1s2 | 2.68E−06 | −0.192088243 | 0.54 | 0.629 | 0.004249565 |
| Ascl1 | 2.74E−06 | 0.167511995 | 0.628 | 0.885 | 0.004336722 |
| Hacd2 | 2.88E−06 | −0.151874561 | 0.569 | 0.681 | 0.00455548 |

Control tanycytes also expressed higher levels of many genes selectively expressed in both mature, quiescent tanycytes and retinal Müller glia, whose expression is downregulated following cell-specific deletion of NFIs (15). These include genes that are highly and selectively expressed in mature alpha2 tanycytes, such as Apoe and Kcnj10, the Notch pathway target Hes1, the Wnt inhibitor Frzb, and the transcription factors Klf6 and Bhlh40. Nfia/b/x-deficient tanycytes, in contrast, upregulated Shh, the Notch inhibitor Dlk1, the BMP inhibitor Fst, the neurogenic factors Ascl1 and Sox4, and the Notch pathway target Hes5 (FIG. 2E). Alpha1 and beta1 tanycytes also showed reduced expression of Tgfb2 and Fgf18 (FIG. 11), which were previously shown to be strongly expressed in these cells (23, 27). To validate these results, multiplexed smfISH (HiPlex, ACD Bio-Techne) was conducted, and observed strong upregulation of Shh, along with decreased Frzb expression, in Pdzph1-positive alpha2 tanycytes at P45 in TKO mice (FIG. 2F).

To infer cell lineage relationships between specific tanycyte subtypes and tanycyte-derived neural progenitors, RNA velocity analysis was conducted (28) on the full aggregated scRNA-Seq dataset (FIG. 2G). Alpha2 tanycytes gave rise to proliferating tanycytes, which in turn give rise to neural precursors following cell cycle exit (FIG. 2G, insets). It is noteworthy here that astrocytes appear to arise directly from alpha1 and alpha2 tanycytes, without going through a clear proliferative stage (FIG. 2G, FIG. 2H).

Pseudo-time analysis was used to identify six major temporally dynamic patterns of gene expression that occurred during the process of alpha2 tanycyte-derived neurogenesis (FIG. 2I, Table ST5).

Based on the GO term enriched in each cluster (FIG. 2J), transition from a quiescent to an actively proliferating state is associated with downregulation of metabolic genes (Glul), ion channels (Kcnj10), transcription factors (Lhx2) and Notch pathway components (Notch1)—all of which are expressed at high levels in mature tanycytes (3, 10). In addition, genes regulating ciliogenesis (Dnah7a, Cfap65) are rapidly downregulated. Following upregulation of genes controlling cell cycle progression and DNA replication (Cenpf; Mcm3), and cell cycle exit (Btg2), tanycyte-derived neural precursors upregulate genes that control chromatin conformation (Phf3), RNA splicing (Sf3b1), and neurogenesis (Hes6). This upregulation is then followed by expression of transcription factors that control specification of specific hypothalamic neuronal subtypes (Dlx1, Lhx6) and regulators of synaptogenesis (Erbb4), neurotransmitter biogenesis and reuptake (Gad1, Pdyn, Slc32a1), neurotransmitter receptors (Grin1, Gria2), and leptin signaling (Lepr).

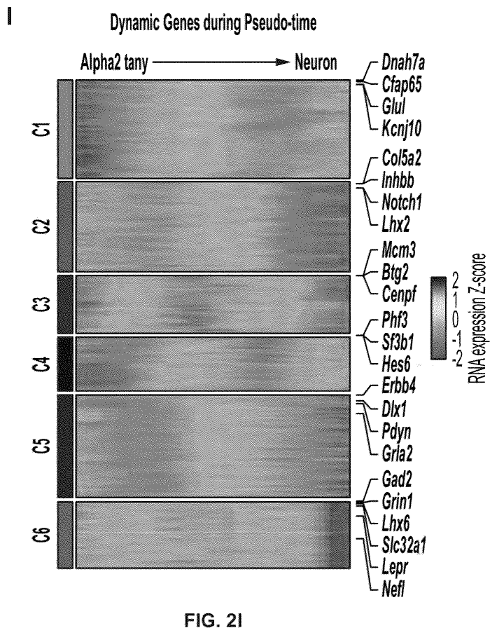

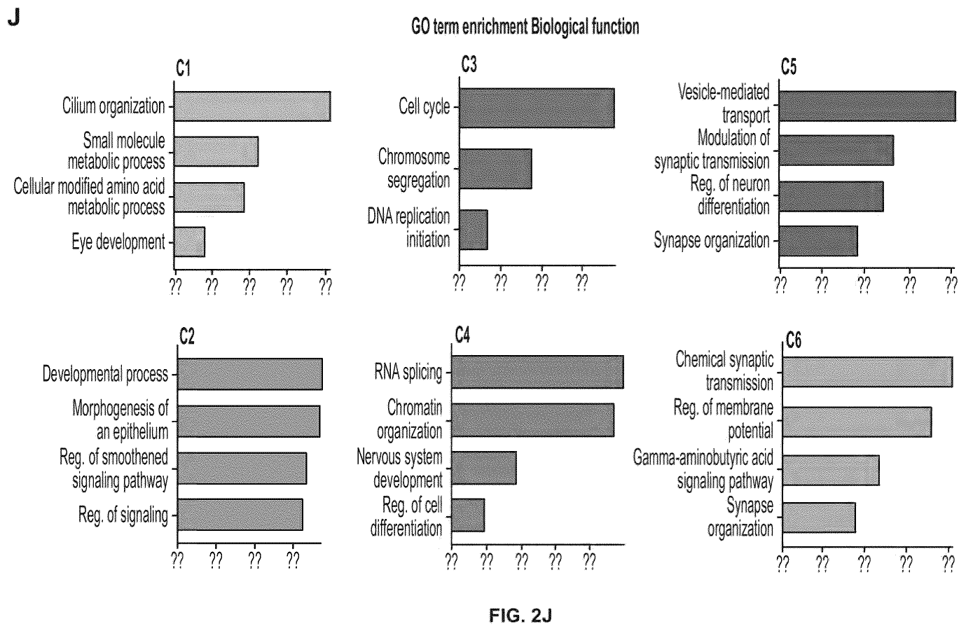

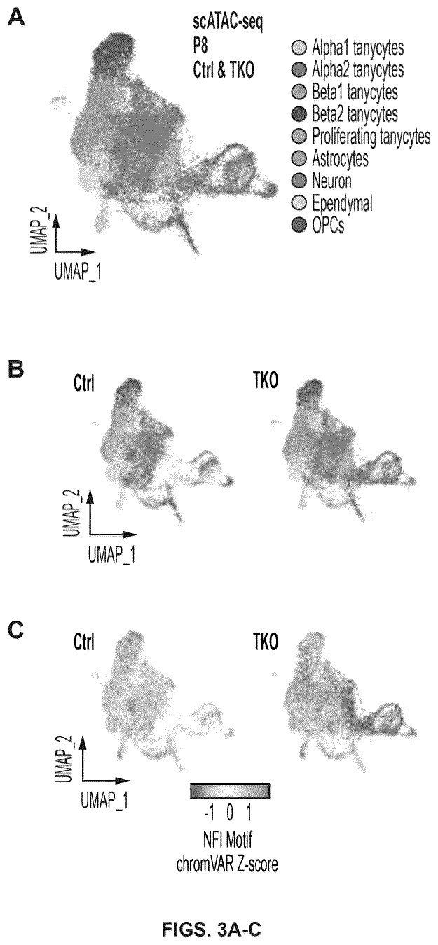

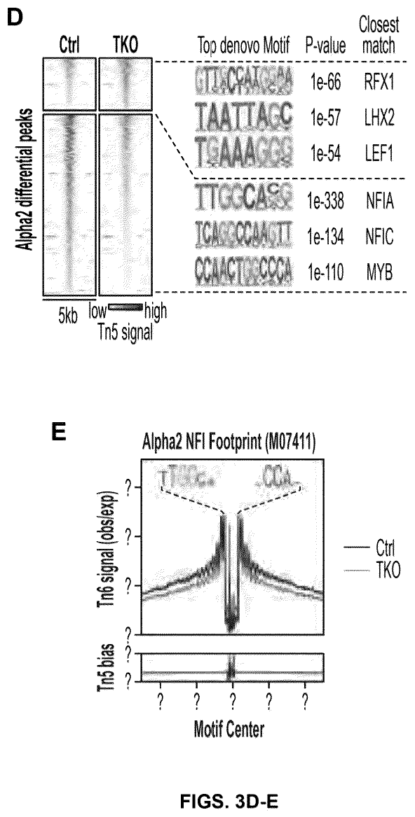

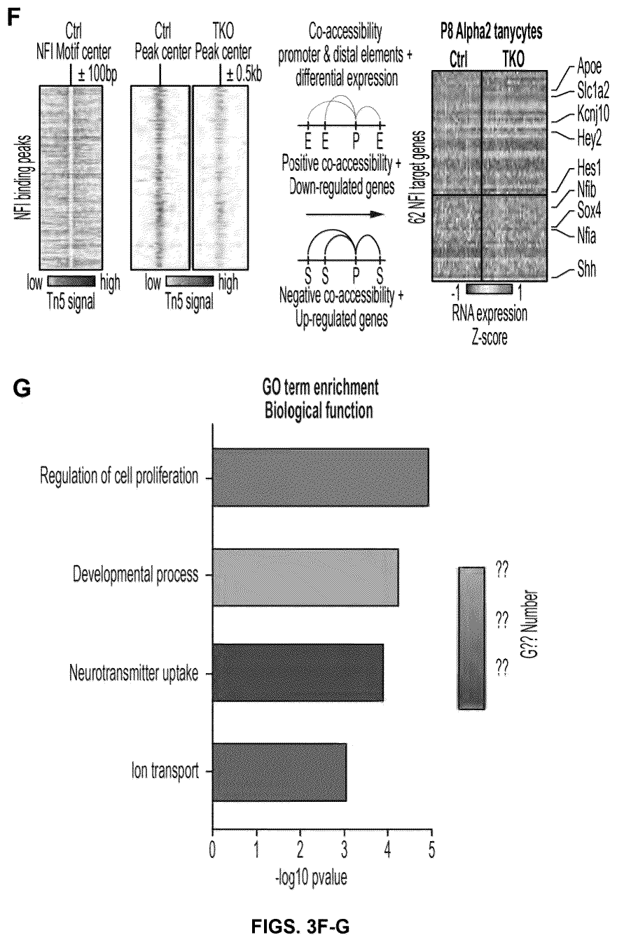

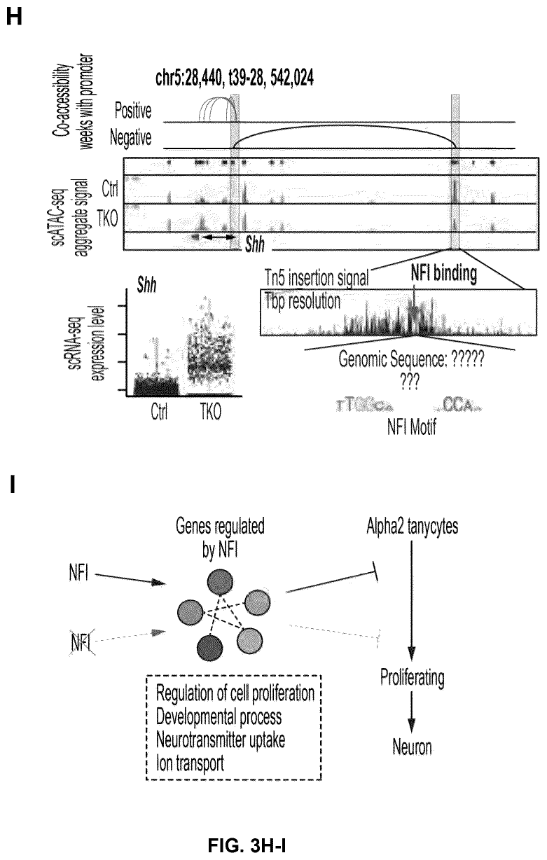

To explore the gene regulatory networks that modulate neurogenic competence in alpha2 tanycytes, scATAC-Seq analysis was conducted in FACS-isolated GFP-positive tanycytes and tanycyte-derived cells in both control and TKO mice at P8. UMAP analysis indicated that cell identity in both control and mutant samples could be readily assigned, based on gene expression data obtained from scRNA-Seq (FIG. 3A). The overall distribution of cell types was much like that seen for scRNA-Seq data (FIG. 2A), with more proliferating tanycytes and tanycyte-derived neurons observed in TKO cells compared to controls (FIG. 3B). Accessibility of the consensus NFI motif was assessed in all cell types in Nfia/b/x-deficient mice (FIG. 3C), and reduced levels of bound transcription factors were observed at these sites by footprinting analysis (FIG. 3E), indicating that Nfia/b/x are actively required to maintain accessible chromatin at a subset of their target sites.

639 chromatin regions were observed that showed increased, and 3072 regions that showed decreased, accessibility in Nfia/b/x-deficient alpha 2 tanycytes relative to controls (FIG. 3D). As expected, HOMER analysis indicated that open chromatin regions (OCRs) specific to controls were most highly enriched for consensus sites for NFI family members (FIG. 3D, Table ST6).

In contrast, motifs for the Wnt effector Lef1 were enriched at OCRs specifically detected in Nfia/b/x-deficient alpha2 tanycytes. A subset of genes with altered expression in scRNA-Seq showed altered accessibility in putative associated cis-regulatory sequences, although changes in gene expression and chromatin accessibility often diverged (Table ST7, ST8). While putative regulatory elements of the downregulated genes Aqp4, Hes1, and Fgf18 showed reduced accessibility, elements associated with other downregulated genes such as Tgfb2 and Sox8 showed increased accessibility. Likewise, while most upregulated genes showed increased accessibility, such as Shh and Sox4, some showed decreased accessibility. These divergent responses indicate that NFI genes control expression of a large number of transcription factors in tanycytes, which appear to perform dual roles as both activators and repressors.

To identify direct targets of NFI factors, and to better clarify their function in regulating proliferation and neurogenesis in alpha2 tanycytes, scRNA- and scATAC-Seq data was integrated from alpha2 tanycytes to identify genes with both altered expression and altered accessibility at sites containing NFI consensus sequences, identifying 62 genes in total (FIG. 3F, Table ST9). These include downregulated genes such as Kcnj10, Apoe and Notch pathway effectors such as Hes1 and Hey2, as well as Shh and Sox4, which are upregulated, with direct target genes enriched for genes controlling proliferation and neural development (FIG. 3G). Transcriptions of Nfia and Nfib are themselves strongly activated by Nfia/b/x, consistent with findings in retina (14, 15)). Importantly, NFI binding sites were found in peaks which are negatively correlated with the promoter of Shh, suggesting that NFI may directly repress Shh expression (FIG. 3H). Thus, NFI factors may act as both activators and repressors in alpha2 tanycytes that promote quiescence while inhibiting proliferation and neurogenesis (FIG. 3I).

### Example 4: Shh and Wnt Signaling Regulated Tanycyte Proliferation and Neurogenic Competence

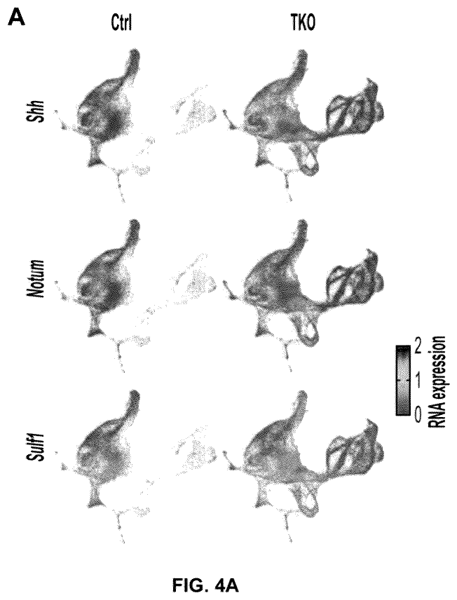

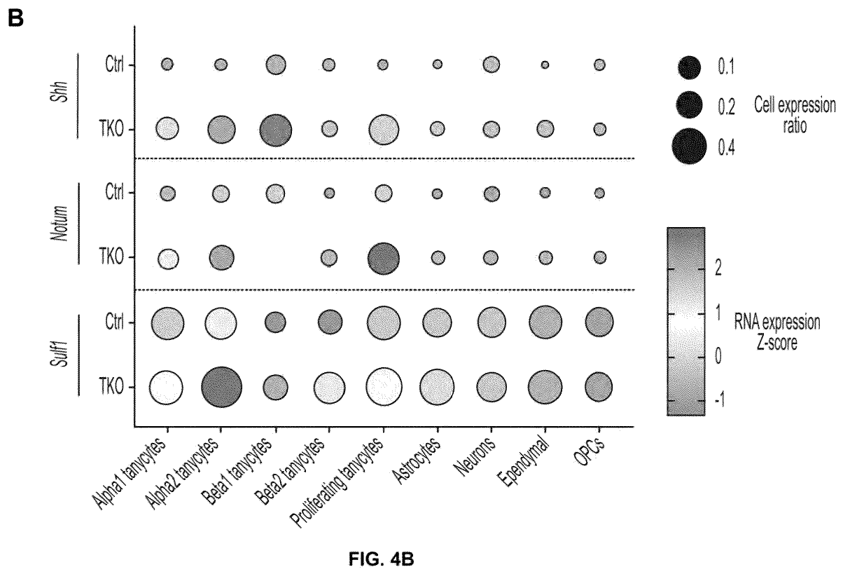

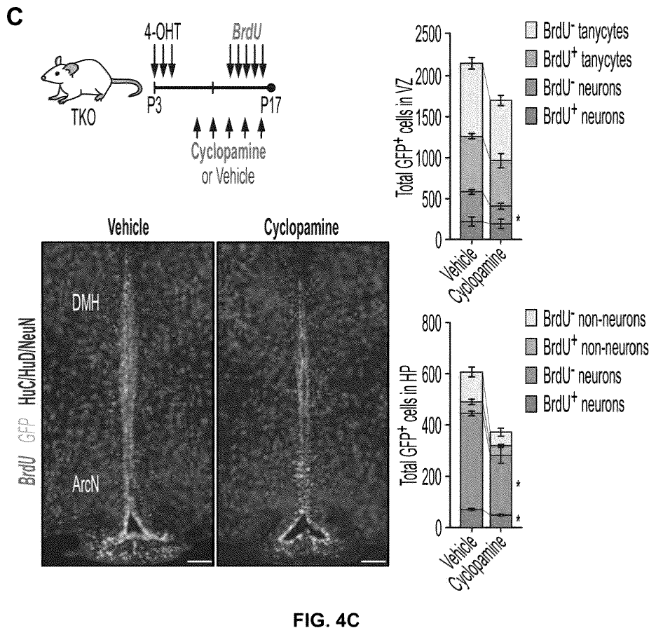

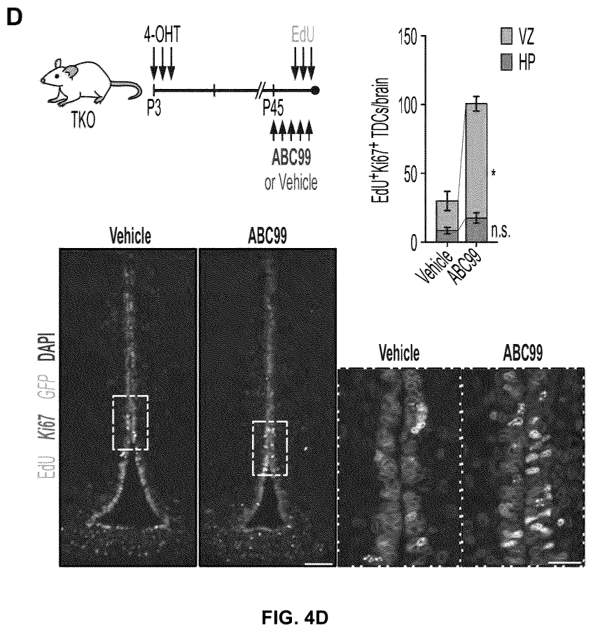

The increased expression of Shh and Wnt regulators that is observed in Nfia/b/x-deficient alpha2 tanycytes (FIG. 2E, Table ST4, FIG. 4A and FIG. 4B) suggested that Shh and Wnt signaling might promote proliferation and/or neurogenesis in tanycytes. Substantially increased expression of Shh in both alpha2 and beta1 tanycytes (FIG. 2F and FIG. 11) was observed, and more complex regulation of Wnt signaling modulators. Increased expression of Sulf1 was observed, which by regulating synthesis of heparan sulfate proteoglycans, typically enhances Wnt signaling (29). However, the broad-spectrum Wnt inhibitor Notum, which was recently shown to regulate quiescence in adult neural stem cells of the lateral ventricles (30), is upregulated from P17, potentially acting in a cell-autonomous manner counteracting the effects of increased cellular levels of Wnt signaling (FIGS. 4A and 4B).

To determine whether Shh inhibition might inhibit tanycyte proliferation and/or tanycyte-derived neurogenesis, the blood-brain barrier-permeable Shh antagonist cyclopamine was administered via i.p. injection to Nfia/b/x-deficient mice every 2 days from P8 until P16, in conjunction with daily i.p. injections of BrdU from P12 to P16 (FIG. 4C). At these ages, Shh is both highly expressed in tanycytes and levels of proliferation and neurogenesis are high in Nfia/b/x-deficient alpha2 tanycytes. Cyclopamine administration resulted in a significant reduction in both the numbers of total GFP-positive cells and GFP/NeuN double-positive neurons in both the tanycytic layer in ventricular zone (VZ) and hypothalamic parenchyma (HP) compared to vehicle controls, while BrdU incorporation was only significantly different in parenchymal neurons, indicating a stronger effect on tanycyte-derived neurogenesis than on self-renewing patterns of proliferation (FIG. 4C).

To determine whether Notum-dependent Wnt signaling played a role in inhibiting tanycyte proliferation and/or neurogenesis at later ages, P45 Nfia/b/x-deficient mice were treated with the blood-brain barrier-permeable Notum inhibitor ABC99 (31) once daily for 5 days, with EdU co-administered on the last 3 days. At this age, Notum expression is high and levels of tanycyte proliferation are substantially reduced relative to the early postnatal period. This led to a significant increase in proliferation in alpha2 tanycytes (FIG. 4D), indicating that Wnt inhibition stimulates tanycyte proliferation.

### Example 5: Nfia/b/x-Deficient Tanycytes Give Rise to a Diverse Range of Hypothalamic Neuronal Subtypes

To investigate the identity of tanycyte-derived neurons (TDNs), a neuronal subset of scRNA-Seq data obtained from both control and Nfia/b/x-deficient mice (FIG. 5A) was analyzed. A total of 582 neurons were obtained from controls, while 15,489 neurons were obtained from Nfia/b/x-deficient mice (Table ST2). The great majority of control tanycyte-derived neurons were obtained at P8, while large numbers of tanycyte-derived neurons were seen at all ages in Nfia/b/x-deficient mice. UMAP analysis revealed that both control and Nfia/b/x-deficient tanycyte-derived neurons fell into two major clusters each of glutamatergic and GABAergic subtypes, with additional clusters corresponding to Ascl1/Hes5-positive postmitotic neural progenitor cells and immature Dlx1/Sox11-positive GABAergic precursors (FIGS. 5A and 5B).

RNA velocity analysis indicated three distinct major differentiation trajectories in both control and Nfia/b/x-deficient tanycyte-derived neurons, which give rise to the two major glutamatergic clusters and the GABAergic neurons (FIG. 5C).

53% of tanycyte-derived neurons were GABAergic—as determined by expression of Gad1, Gad2 and/or Slc32a1—while 30% were Slc17a6-positive glutamatergic neurons (FIG. 5B, Table ST10, ST11). Glu_1 was enriched for the transcription factors Nhlh2 and Neurod2, as well as markers of glutamatergic VMH neurons, such as Nr5a1, Cnr1, and the androgen receptor Ar, while Glu_2 was enriched for markers of glutamatergic DMH neurons, such as Ppp1r17, and ArcN markers such as Chgb (FIG. 5B). GABAergic neurons expressed a diverse collection of molecular markers expressed by neurons in the ArcN and DMH, as well as the adjacent zona incerta (ZI), which regulates a broad range of internal behaviors including feeding, sleep and defensive behaviors (32). GABA_1 was enriched for a subset of ZI and DMH-enriched genes (Lhx6, Pnoc), while GABA_2 was enriched for genes selectively expressed in ArcN neurons (Isl1), as well as genes expressed in GABAergic neurons in both the ArcN and DMH (Cartpt, Npy, Sst, Gal, Trh, Th).

Multiplexed smflSH (FIGS. 5D and 5F) and immunohistochemistry (FIG. 5E, ST12) was used to confirm expression of Th, Lhx6, and Gal in GFP-positive Nfia/b/x-deficient tanycyte-derived GABAergic neurons in the dorsomedial hypothalamus, as verified by Egfp Slc32a1 (or Gad1) co-labeling. Expression of Cartpt and Th was observed in GABAergic tanycyte-derived neurons in both the ArcN and DMH (FIG. 5G and FIG. 12A), and co-expression of Lhx6 and Th, as well as Cartpt and Th, was observed using immunohistochemistry in a subset of tanycyte-derived and non-tanycyte-derived neurons. Small numbers of Nr5a1-expressing glutamatergic (Slc17a6-positive) neurons were detected in the dorsomedial VMH (FIG. 5H). No expression of markers specific to neurons of more anterior or posterior hypothalamic regions (e.g. Avp, Crh, Oxt, Pmch, Hcrt, Vip, etc) was detected (Table ST 11).

These data demonstrate that tanycyte-derived neurons express molecular markers of multiple neuronal subtypes located in the tuberal hypothalamus, including the ArcN and VMH. To determine how closely the gene expression profile of tanycyte-derived neurons more broadly resembles the profile of hypothalamic neurons in these regions, LIGER analysis was used (33) to integrate clustered scRNA-Seq from a previous study of adult ArcN, in which a small number of VMH neurons was also profiled (23) (FIG. 12B and Table ST13). Integration of these datasets using LIGER (34), and comparison of cell types in each cluster using alluvial plotting (FIG. 12C), indicate substantial overlap in cellular gene expression profiles between glutamatergic tanycyte-derived neurons cluster Glu_1 with both Kiss1/Tac2-positive ArcN neurons and Nr5a1-positive VMH neurons. Glu_2 overlapped with Rgs16/Nmu-positive ArcN neurons. GABAergic tanycyte-derived GABA_1 and GABA_2 clusters overlapped with several different ArcN neuronal clusters, including clusters that contained neurons expressing Th, Ghrh and/or Trh. In contrast, some subtypes of ArcN neurons were represented only sparsely or not at all among tanycyte-derived neurons, while other tanycyte-derived neurons appeared to correspond to cell types not found in the published scRNA-Seq dataset. For instance, while some tanycyte-derived neurons closely resembled Pomc-expressing ArcN neurons, the relative fraction of Pomc-positive tanycyte-derived neurons was substantially lower than in ArcN (LIGER Cluster 4). Likewise, no tanycyte-derived neurons mapped to LIGER Cluster 7, which corresponded to Agrp-positive ArcN neurons, and no Agrp-positive tanycyte-derived neurons were detected using smfISH (FIG. 5G and FIGS. 12A-12C). While, as expected, few immature tanycyte-derived neurons mapped to neurons in the mature ArcN scRNA-Seq dataset, two clusters of mature glutamatergic (LIGER Clusters 8 and 9) and one cluster of GABAergic (LIGER Cluster 11) also showed little correspondence to ArcN neurons, and appeared to correspond to DMH-like neurons based on their expression of DMH-enriched genes (e.g. Ppp1r17, Lhx6).

At P45, 4.4% of tanycyte-derived neurons in the GABA_2 cluster expressed the leptin receptor Lepr (FIG. 5B), despite Lepr being essentially undetectable in tanycytes themselves, as previously reported (19). As a result, it was tested whether tanycyte-derived neurons were capable of responding to leptin signaling. P90 mice that had undergone an overnight fast were injected i.p. with 3 mg/kg leptin, and sacrificed after 45 minutes, providing sufficient time for leptin-responsive neurons throughout the hypothalamus to induce pStat3 immunoreactivity (35, 36). Robust induction of pStat3 immunoreactivity in GFP-positive tanycyte-derived neurons under these conditions (FIG. 5I) was observed, with low levels of immunoreactivity under unstimulated conditions, as previously reported (35, 36). A total of 42.3 (2.5) % of parenchymal tanycyte-derived neurons in DMH and ArcN show pStat3 induction under these conditions, along with 22.3 (±3.9) % of tanycyte-derived neurons in the subventricular region (FIG. 5J), confirming that a subset of tanycyte-derived neurons are leptin-responsive.

### Example 6: Neurons Derived from Nfia/b/x-Deficient Tanycytes Integrate into Hypothalamic Neural Circuitry

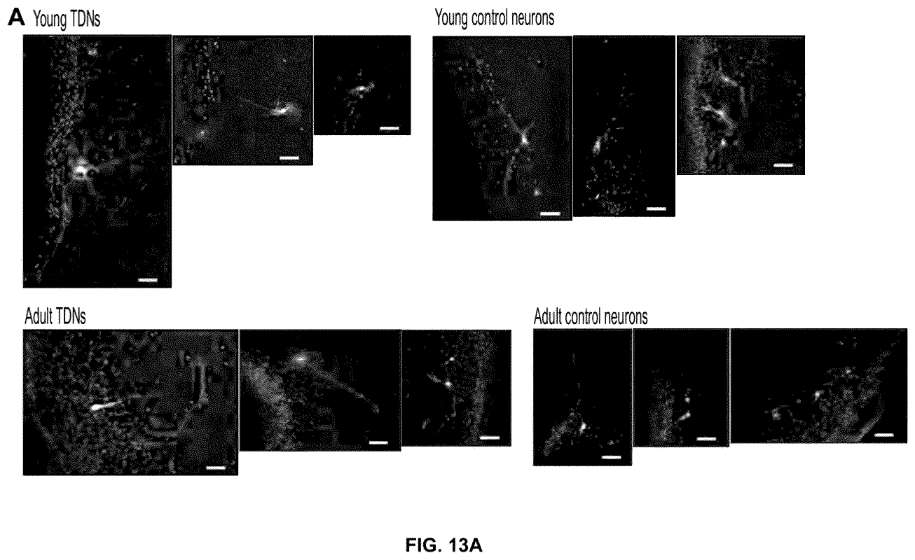

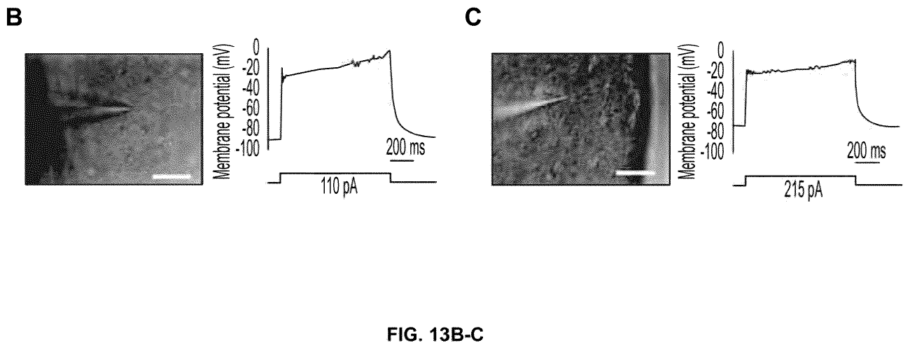

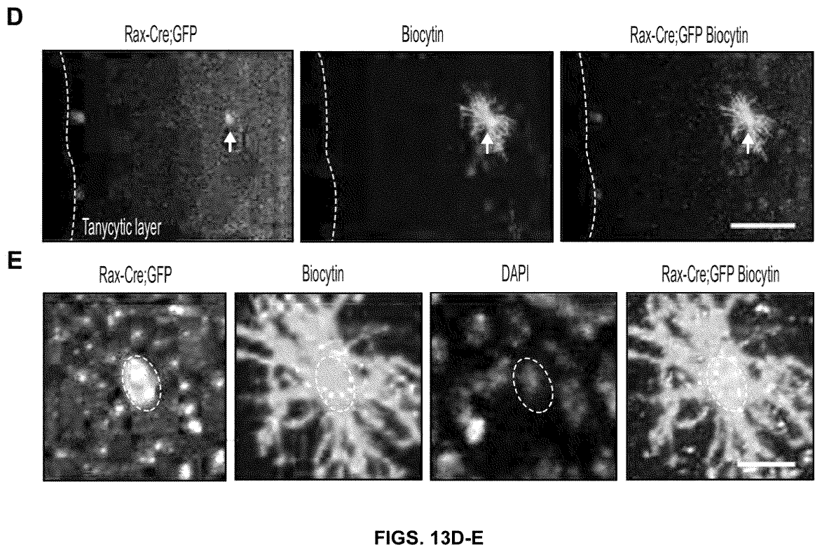

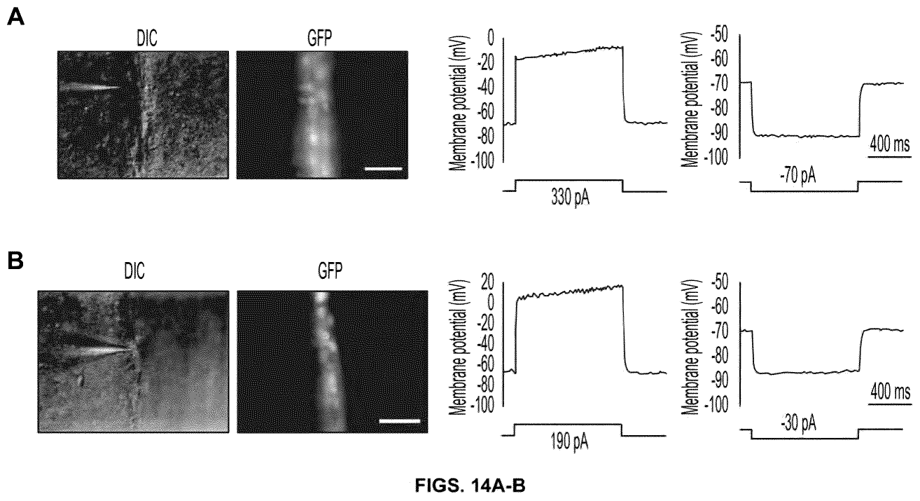

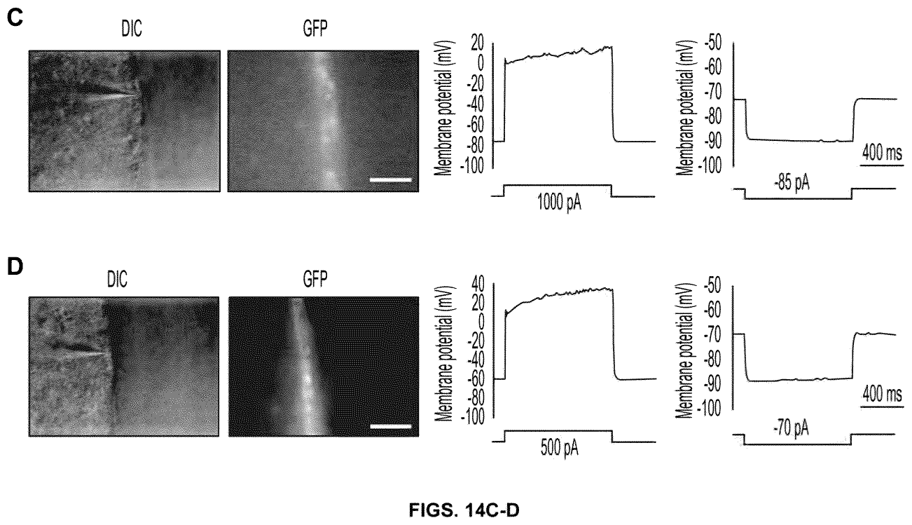

Since the scRNA-Seq analysis identified the majority of tanycyte-derived cells as neurons in Nfia/b/x-deficient mice, it next investigated whether these cells showed electrophysiological properties of functional neurons. To characterize tanycyte-derived cells, whole-cell patch-clamp recordings were performed from GFP-positive parenchymal cells in acute brain slices obtained from Nfia/b/x-deficient mice that had undergone 4-OHT treatment between P3 and P5. Biocytin filling of recorded tanycyte-derived cells revealed neuron-like morphology, typically showing 3-5 major dendritic processes, similar to GFP-negative control neurons (FIG. 6A, FIGS. 13A-13E). The majority of GFP-positive tanycyte-derived cells in the hypothalamic parenchyma fired action potentials in response to depolarizing current steps (FIG. 6B and FIG. 6C, 95%, 40 of 42 cells from P15-P97). In contrast, GFP-positive cells located in the tanycytic layer retained non-spiking, glial-like electrophysiological properties in Nfia/b/x-deficient mice (FIGS. 14A-14D).

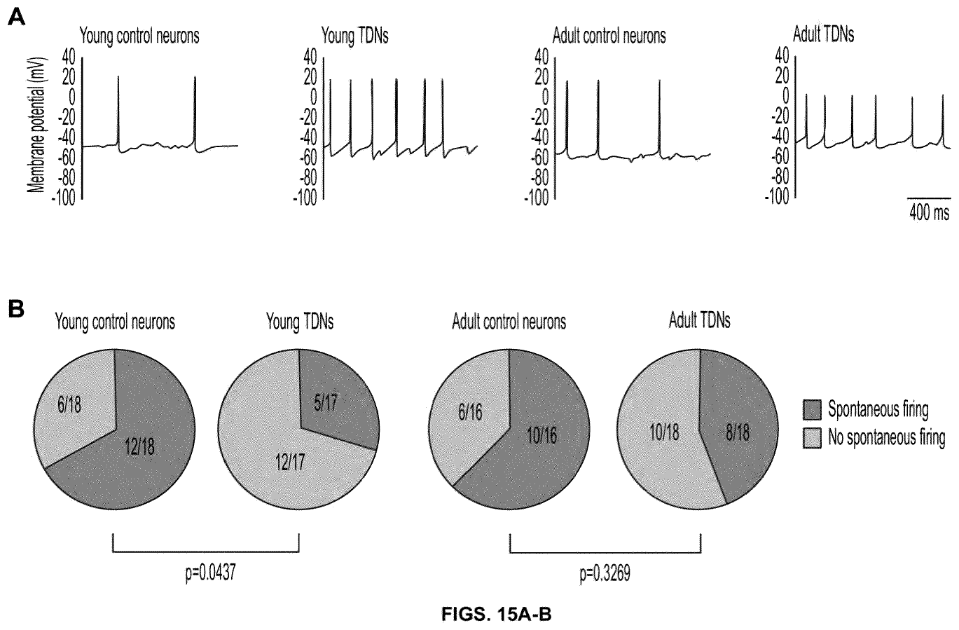

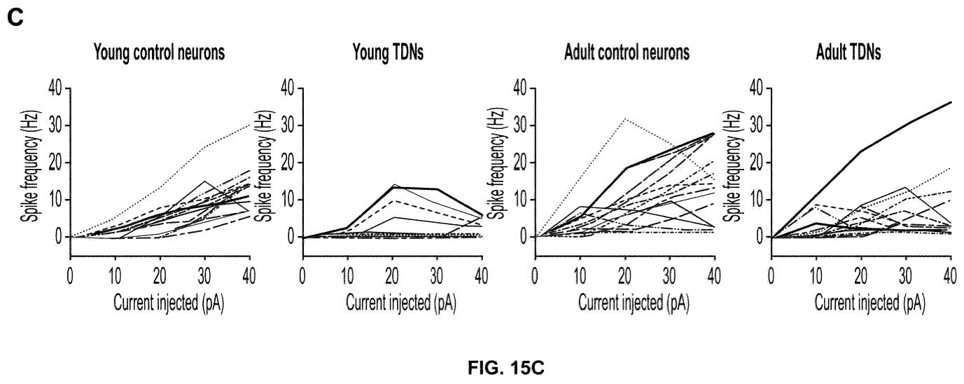

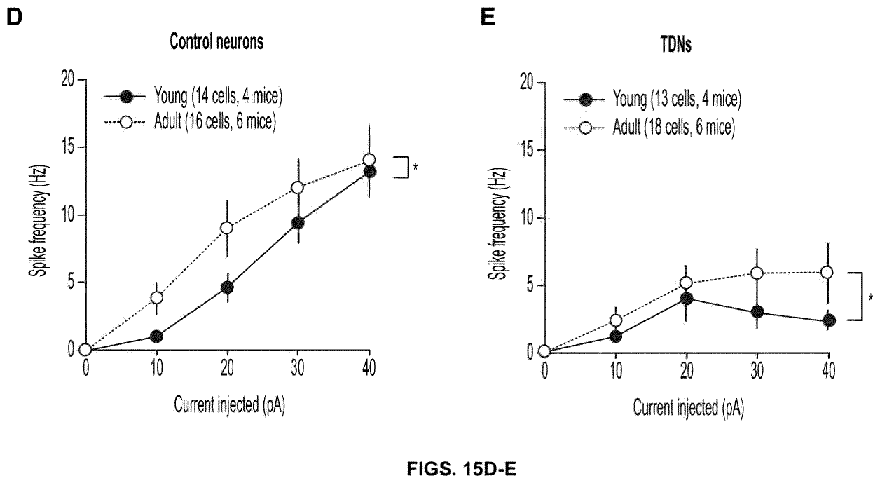

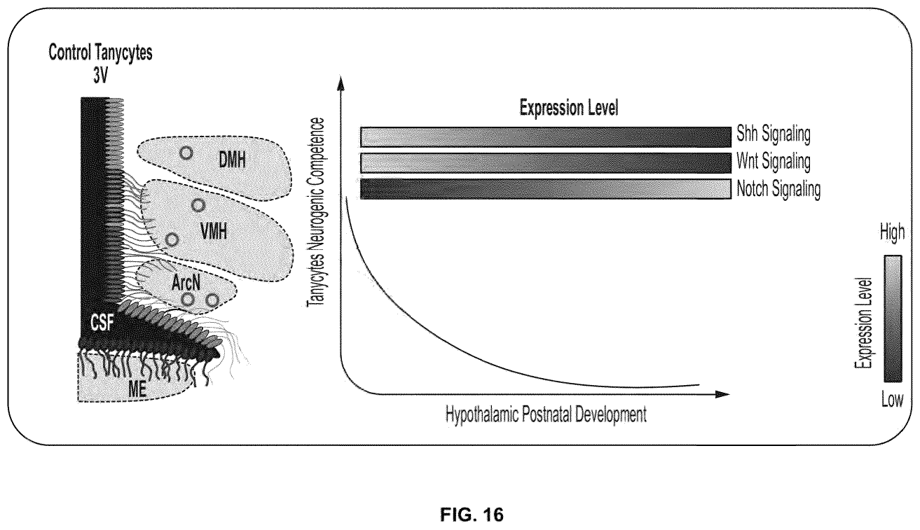

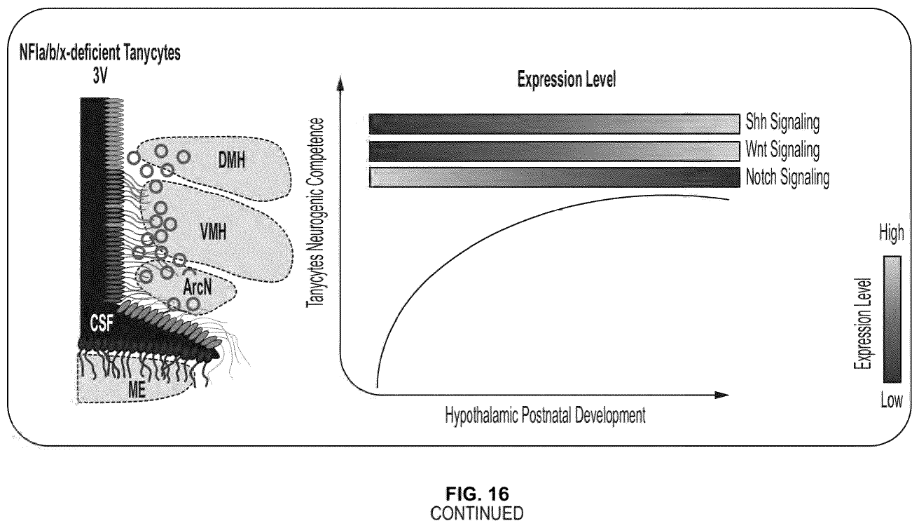

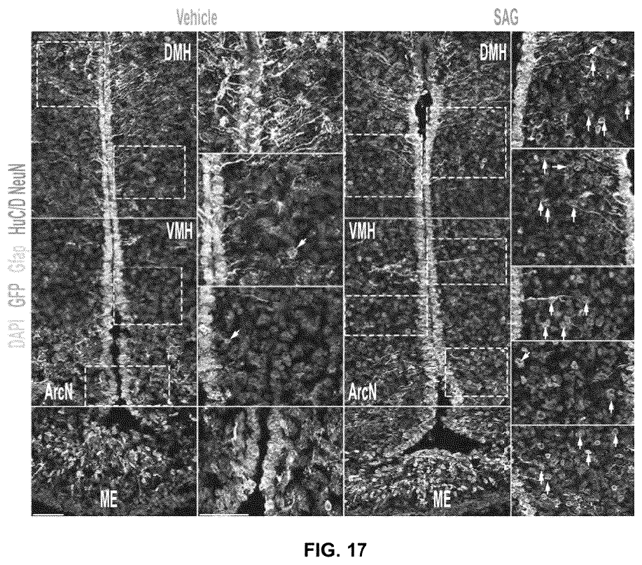

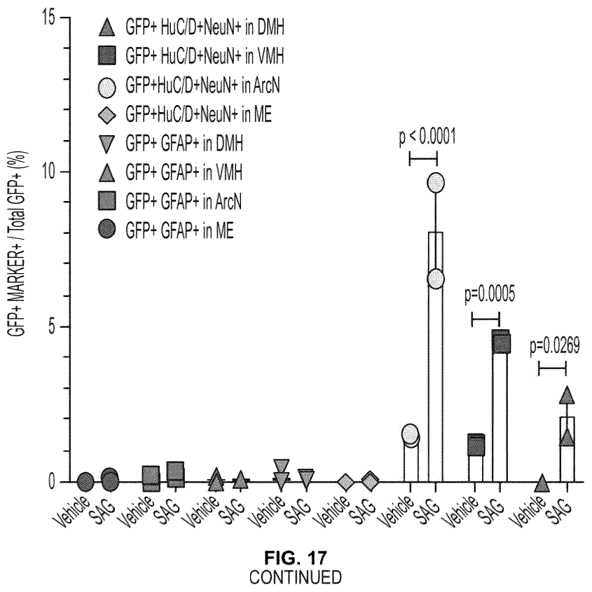

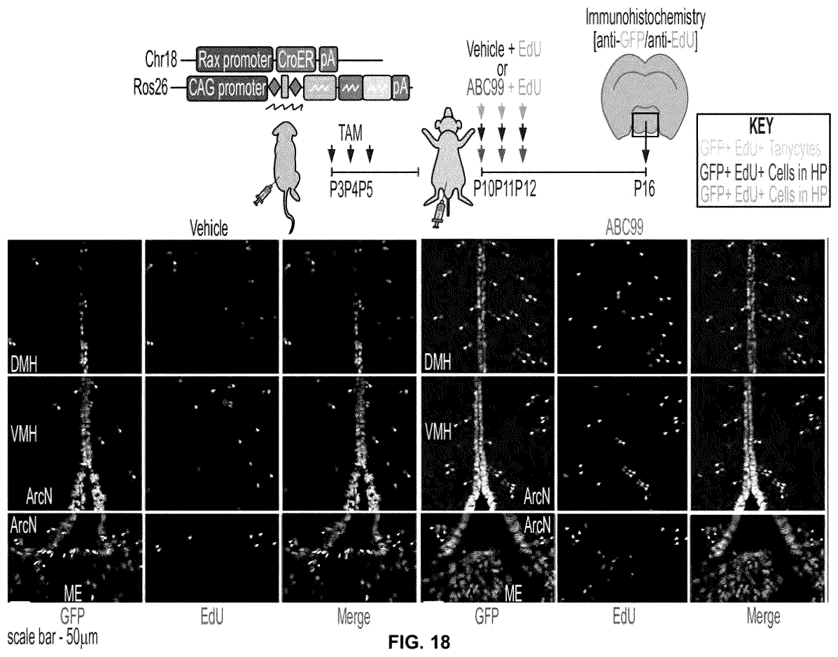

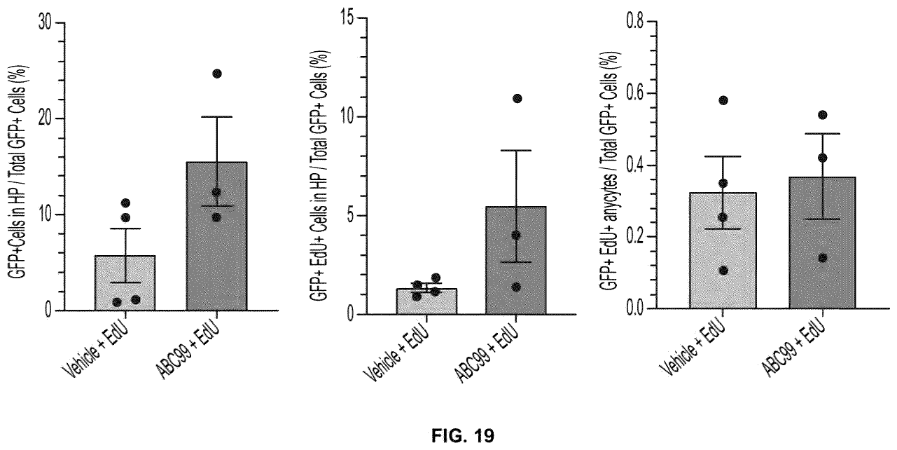

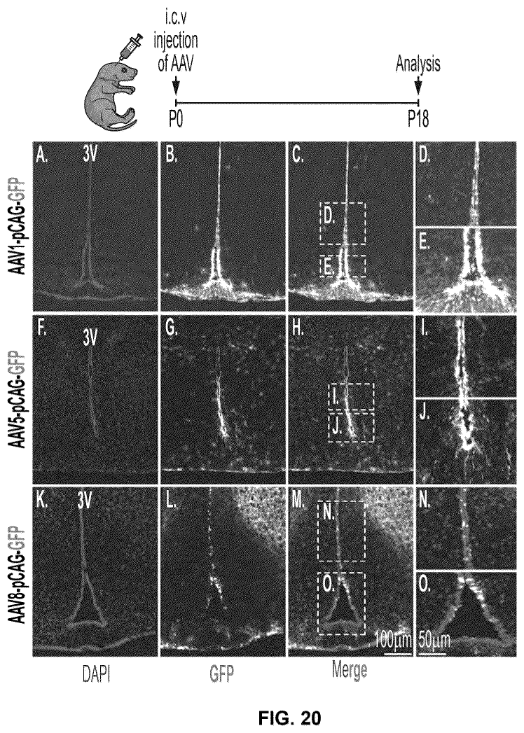

Like typical hypothalamic neurons (37), many tanycyte-derived neurons fired spontaneous action potentials (sAPs) and exhibited relatively depolarized resting membrane potentials (FIGS. 15A-15E). However, the proportion of tanycyte-derived neurons showing sAPs was significantly lower than that of GFP-negative control neurons in young (P15-17) mice (FIG. 15B). In addition, the input resistance was significantly higher in tanycyte-derived neurons than in GFP-negative control neurons in young (P15-19) mice, although the input resistances were similar for tanycyte-derived and control neurons in adult mice (P86-P97) (FIGS. 6D and 6E, Table ST14). Together, these data indicate that, although there were some differences with GFP-negative control neurons, almost all tanycyte-derived cells fired action potentials and shared electrophysiological features of control neurons, indicating that they are indeed neurons.

Since the great majority of tanycyte-derived cells thus appear to be functional neurons, it was next asked whether their evoked action potential firing properties were similar to those of neighboring GFP-negative hypothalamic neurons. Although tanycyte-derived neurons fired action potentials to depolarizing current steps, the average current-frequency curve of tanycyte-derived neurons was significantly different from the curve for control neurons in both young and adult mice (FIGS. 6F and 6G). Although the number of action potentials elicited initially increased with age, the tanycyte-derived neurons were unable to reliably generate repetitive actions potentials with larger current steps in contrast to control neurons, leading to saturation of the current-frequency curves (FIG. 6G; FIGS. 15C and 15D). These results suggest that tanycyte-derived neurons may have a different ion channel composition than control neurons and may not be as developmentally mature.

It was next asked whether tanycyte-derived neurons receive synaptic inputs from other neurons and are functionally integrated into hypothalamic circuits. Spontaneous postsynaptic currents (sPSCs) were detected in 34 of 35 recorded tanycyte-derived neurons (FIGS. 6H and 6I). However, the frequency of sPSCs in adult tanycyte-derived neurons was significantly lower than for control neurons in adult mice (FIGS. 6H, and 6I, and Table ST14), suggesting that the number of functional synapses received is nonetheless fewer than for wild type neurons. As the histological data suggest that tanycyte-derived neurons undergo progressive radial migration away from the tanycyte layer into the hypothalamic parenchyma as they mature, it was asked whether there was any correlation between a tanycyte-derived neuron's distance from the tanycyte layer and number of synaptic inputs it receives. A positive correlation between a tanycyte-derived neuron's distance from the tanycyte layer and the sPSC frequency was observed in both young and adult mice, suggesting that the farther a tanycyte-derived neuron migrates from the tanycytic layer, the more functional synapses from local neurons it receives (FIG. 6J).

Having established that tanycyte-derived neurons integrate into hypothalamic circuits, next, it was tested whether tanycyte-derived neurons are activated in response to changes in internal states that modulate the activity of nearby hypothalamic neurons. The response of tanycyte-derived neurons in the DMH to heat stress was investigated, which is known to robustly modulate the activity of DMH neurons (38, 39). At P45, 16.0±1.6% of parenchymal tanycyte-derived neurons in the DMH induce c-fos expression following 4 hrs of exposure to 38° C. ambient temperature in Nfia/b/x-deficient mice, which is essentially equivalent to the portion of GFP-negative control neurons activated, 13.7±0.3% (FIGS. 6K and 6L) was observed.

### Materials and Methods

The following materials and methods were used.

### Animals

Rax-CreER^{T2} mice (The Jackson Laboratory Stock No. 025521) generated in the laboratory (18) were crossed with the Cre-inducible Sun1-GFP reporter (20) (Sun1-sfGFP-Myc, The Jackson Laboratory Stock No. 021039, provided by Jeremy Nathans). Nfia^{lox/lox} (14, 15), Nfib^{lox/lox} (62), and Nfix^{lox/lox} (63) mice were used to generate Nfia/b/x homozygous triple mutant mice previously described (14, 15). To generate tanycyte-specific loss of function mutants of Nfia/b/x genes, the triple mutant mice were crossed to Rax-CreER^{T2}; Sun1-GFP mice. To induce Cre recombination, these mice were intraperitoneally (i.p.) injected with 0.2 mg 4-hydroxytamoxifen (4-OHT) dissolved in corn oil for 3 consecutive days from postnatal day (P) 3 to P5. Mice were housed on a 14 hr-10 hr light/dark cycle (07:00 lights on-21:00 lights off) in a climate-controlled pathogen-free facility. All experimental procedures were preapproved by the Institutional Animal Care and Use Committee (IACUC) of the Johns Hopkins University School of Medicine.

### BrdU and EdU Incorporation Assay

To label the proliferating cells, BrdU (Sigma #B5002) was dissolved in saline solution and 100 mg/kg of body weight was i.p. injected for 5 consecutive days for the dates indicated. For the AAV1-Cre-mCherry stereotaxic injection, an osmotic mini-pump (Alzet model 1002, #0004317) was filled with BrdU dissolved in aCSF (TOCRIS #3525) and installed immediately into the hole remaining after the virus injection needle was removed. The 2 cm long tube connecting the mini-pump and cannula was filled with aCSF, so that the actual 30 μg/day infusion of BrdU was started from the third day following implantation. To avoid potential toxic effects of long-term BrdU exposure, EdU (ThermoFisher #A10044) was used for quantitative studies of cell proliferation. For this purpose, a 50 μg/g dose of EdU was used, which has been previously validated for proliferation studies in the adult brain (64).

### Inhibitory Drug Administration

ABC99 (Sigma #SML2410) was prepared as previously described (65), except for the fact that a 16.5 mg/ml stock solution was used. This stock was sequentially mixed with Tween-80 (Sigma #P1754), PEG-400 (Merch #91893), and 0.9% NaCl in the ratio of 1:1:1:17. P45 Nfia/b/x triple knockout male mice were i.p. injected with 10 mg/kg ABC99 for 5 consecutive days. 50 mg/kg body weight EdU was injected together from the third day of treatment in order to profile proliferation induced by Notum inhibition (30). For the 1 μg/μl Cyclopamine stock solution, 1 mg Cyclopamine (TOCRIS, #1623) powder was dissolved into 1 ml 2-hydroxylpropyl-beta-cyclodextrin (Sigma #C0926) prepared as a 45% solution in phosphate buffered saline (PBS). 10 μg/g Cyclopamine was injected from P8 to P16 on alternative days. 100 μg/g body weight BrdU was injected together from the 3rd injection for 5 consecutive days. Control mice were treated with the corresponding vehicle solution.

### Tissue Processing and Immunohistochemistry

Mice were anesthetized with an i.p. injection of Tribromoethanol/Avertin, followed by transcardial perfusion with 2% paraformaldehyde (PFA) as previously described (66). Brains were dissected, postfixed in the same fixative, and prepared for the cryopreservation in O.C.T. embedding compound. A series of 25 μm coronal sections were stored in antifreeze solution at −20° C. until ready for immunostaining.

After brief washing with 1×PBS to remove the antifreeze solution, sections were mounted on Superfrost Plus slides (Fisher Scientific) before starting the immunohistochemistry and dried at room temperature for 30 min. To ensure adequate fixation for nuclear staining, sections were immersed into the fixative solution for 10 min at this point. Antigen retrieval was performed by incubating the slides with the prewarmed sodium citrate buffer (10 mM sodium citrate, pH 6.0) in an 80° C. water bath for 30 min. For HuC/D antibody staining, sections were also treated with 0.3% hydrogen peroxide in order to block endogenous peroxidase activity prior to the blocking step with 10% horse serum/0.1% Triton X-100 in 1×PBS for an hour. pSTAT3 staining required different pretreatment as described before (19, 66). After finishing the first round of immunostaining, the fluorescence signal was crosslinked by incubation in 2% PFA for 10 min, followed by either EdU staining using the Click-iT EdU detection kit conjugated with Alexa Fluor 647 (ThermoFisher #C10340) or BrdU antibody staining. For BrdU staining, freshly prepared 2N HCl was spread on the slides and incubated at 37° C. for 30 min on the humidified chamber. 0.1M Borate buffer (pH 8.5) was used for acid neutralization by incubating for 10 min at room temperature. Antibodies used were listed in Table 1, below. After counter staining with DAPI, the slides were coverslipped with Vectashield antifade mounting medium (Vector Laboratories, #H-1200) and dried at room temperature for no more than 30 min. The slides were stored at 4° C. and imaged within two days to achieve the best quality using a Zeiss LSM 700 Confocal at the Microscope Facility at Johns Hopkins University School of Medicine.

TABLE 1
Resource Table-Antibodies used for IHC

| Reagent | Source | Identifier |
|---|---|---|
| Antibodies | | |
| Rabbit anti-GFP | Invitrogen | # A-6455 |
| Goat anti-GFP | Rockland | #600-101-215 |
| | antibodies&assays | |
| Mouse anti-Lhx6 (clone A-9) | Santa-Cruz | #SC-271433 |
| Rabbit anti-tyrosine | Pel-Freez | #P4010-150 |
| hydroxylase (TH) | | |
| Mouse anti-NeuN | Millipore | #MAB377 |
| Mouse anti-HuC/D(16A11) | Invitrogen | #A-21271 |
| Rat anti-BrdU[BU1/75 | Abcam | #ab6326 |
| (ICR1)] | | |
| Rabbit anti-phospho-c- | Cell Signaling | #5348T |
| Fos(Ser32) | | |
| Rabbit anti-phospho-Stat3 | Cell Signaling | #9145S |
| Chemicals, Peptides, and | | |
| Recombinant Proteins | | |
| ABC99 | Sigma-Aldrich | #SML2410 |
| Cyclopamine | Tocris | #1623-1 mg |
| Cyclopamine | PeproTech | #4445185- |
| | | 10 mg |
| (2-Hydroxypropyl)-beta- | Sigma-Aldrich | #C0926 |
| cyclodextrin | | |
| Recombinant Murine Leptin | PeproTech | #450-31 |
| 4-OHT | Sigma-Aldrich | #H-6278 |
| AAV-CAG-Cre-mCherry | Signagen | #SL101117 |
| (AAV Serotype 1) (SL101117) | | |
| Critical Commercial Assays | | |
| EdU | ThermoFisher | #C10640 |
| Click-iT EdU Alexa Fluor 647 | ThermoFisher | #C10340 |
| Papain dissociation kit | Worthington | #LK003153 |
| Hibernate-A | Gibco | #A1247501 |
| RNAscope ® HiPlex8 Reagent | ACD | #324100 |
| Kit | | |
| GlutaMax | Gibco | #35050061 |
| B-27 | Gibco | #17504044 |
| Experimental Models | | |
| Rax-CreERT2 | The Jackson | #025521 |
| | Laboratory | |
| Nfialox/lox | Clark, et al. 2019 | |
| Nfiblox/lox | Clark, et al. 2019 | |
| Nfixlox/lox | Clark, et al. 2019 | |
| Sun1-lsl-sfGFP | The Jackson | #021039 |
| | Laboratory | |

### RNAscope Hiplex Assay

For the RNAscope Hiplex assay, P45 triple knock-out and control male mice were sacrificed by cervical dislocation and brains were dissected out. The brains were immediately immersed in 4% PFA in DEPC-treated 1×PBS and incubated overnight at 4° C. All other sample preparation procedures were performed as recommended in the manufacturer's instructions for OCT-embedded fresh frozen tissue preparation. 14 μm sections were cut on a cryostat and briefly washed with 1×PBS before mounting on Superfrost Plus slides (Fisher Scientific). The slides were dried at −20° C. and stored at −80° C. before use. The Hiplex assay was performed by following the manufacturer's instructions using probes listed in Table S12. The sections were imaged on a Zeiss LSM 800 Confocal at the Multiphoton Imaging Core in the Department of Neuroscience at Johns Hopkins University School of Medicine.

### Heat Stimulation and Leptin Injection

P45 male mice were exposed to ambient heat (38° C.) for 4 hours (67) by incubating in a prewarmed light-controlled cabinet in the rodent metabolism core facility at the Center for Metabolism and Obesity Research of the Johns Hopkins University School of Medicine. During the procedure, mice were provided ad libitum access to water and food and carefully monitored. Transcardiac perfusion with 4% PFA in 1×PBS was performed immediately after heat exposure. The dissected brains were processed as described above and used for c-fos immunostaining. Leptin injection was performed on P90 male mice that were fasted for 18 hours prior to treatment. 3 mg/kg body weight of leptin (PeproTech, #450-31) dissolved in saline solution was i.p. injected, and 45 min later transcardial perfusion was performed using 2% PFA as described above.

### Cell Counting and Statistical Analysis

All cell counts were performed blindly and manually by five independent observers using Fiji/ImageJ software. Five sections corresponding to −1.55, −1.67, −1.79, −1.91, −2.15 mm from Bregma were chosen among the serial sections for cell counting. Initially, cell numbers were normalized by the size (mm) of hypothalamic nuclei measured. Because Nfia/b/x-deficient animals did not show any obvious structural differences, in subsequent experiments, absolute numbers were used. All values are expressed as mean±S.E.M. Comparisons were analyzed by two-tailed Student's t-test using Microsoft Excel unless stated otherwise. A p-value of <0.05 was considered statistically significant.

Cell Preparation for scRNA-Seq

P8, P17, and P45 Rax-CreER; Nfia^{lox/lox}; Nfib^{lox/lox}; Nfix^{lox/lox}; CAG-lsl-SunU-GFP mutant mice and control Rax-CreER; CAG-lsl-Sun1-GFP mice were sacrificed by cervical dislocation and brains were dissected. One biological replicate of each timepoint and genotype were analyzed, with the exception of P45 TKO, where two biological replicates were analyzed. 2 mm thick coronal slices including the hypothalamic protruding median eminence (ME) were collected using adult mouse brain matrix (Kent Scientific). The mediobasal hypothalamic region was microdissected using a surgical scalpel, dampened in Hibernate-A media supplemented with 0.5 mM GlutaMax and 2% B-27 (HABG) and chopped with a razor blade. Brain tissues were transferred into pre-equilibrated papain/Dnase-I mix (Worthington Papain Dissociation System, #LK003150) and incubated for 30 min at 37° C. with frequent agitation using a fire-polished glass pipette. Dissociated cells were filtered through a 40 μm strainer and subjected to density gradient centrifugation to remove the cell debris as suggested in the manufacturer's protocol. Cells were resuspended in HABG medium and GFP-positive cells were FACS isolated in the Bloomberg Flow Cytometry and Immunology Core at Johns Hopkins University. Cells were resuspended with ice-cold PBS containing 0.04% BSA and 0.5 U/l RNase inhibitor, and 10,000-15,000 cells were loaded on a 10× Genomics Chromium Single Cell system (10× Genomics, Redwood City, CA), using the v3 chemistry following the manufacturer's instructions, and libraries were sequenced on an Illumina NextSeq with ~200 million reads per library. Sequencing results were processed through the Cell Ranger 3.1 pipeline (10× Genomics, Redwood City, CA) with default parameters.

### Single-Cell ATAC-Seq

Single cell ATAC-Seq was performed using the 10× Genomic single cell ATAC reagent V1 kit following the manufacturer's instructions. Briefly, FACS-sorted cells (~30 k cells) were centrifuged at 300×g for 5 min at 4° C. The cell pellet was resuspended in 100 μl of Lysis buffer, mixed 10× by pipetting and incubated on ice for 3 min. Wash buffer (1 ml) was added to the lysed cells, and cell nuclei were centrifuged at 500×g for 5 min at 4° C. The nuclei pellet was resuspended in 250 ul of 1× Nuclei buffer. Cell nuclei were then counted using Trypan blue staining. Re-suspended cell nuclei (10-15 k) were used for transposition and loaded into the 10× Genomics Chromium Single Cell system. Libraries were amplified with 10 PCR cycles and were sequenced on an Illumina NextSeq with ~200 million reads per library. Sequencing data were processed through the Cell Ranger ATAC 1.1.0 pipeline (10× Genomics) with default parameters.

### Single-Cell RNA-Seq Data Preprocessing

Raw scRNA-seq data were processed with the Cell Ranger software (68)(version 3.1) for formatting reads, demultiplexing samples, genomic alignment, and generating the cell-by-gene count matrix. First, the ‘cellranger mkfastq’ function was used to generate fastq files from BCL files. Second, the ‘cellranger count’ function was used to process fastq files for each library using default parameters and the mm10 mouse reference index provided by 10× Genomics. Finally, the cell-by-gene count matrix for each library was obtained, and used this for all downstream analysis.

Using the Seurat (69) R package, Seurat objects were created for each sample with the cell-by-gene count matrix using the function ‘CreateSeuratObject’ (min.cells=3, min.features=200). After visual analysis of the violin plot of the total counts for each cell, cells were filtered out with nCount_RNA <600 or nCount_RNA >6000. Next, the fraction of mitochondrial genes was calculated for each cell and filtered out the cells with a mitochondrial fraction >8%. Finally, multiplet artifacts were predicted and removed potential doublet cells using Scrublet (70) for each sample. As a result, 6609 (P8 Ctrl), 6494 (P17 Ctrl), 2607 (P45 Ctrl), 12930 (P8 TKO), 12413 (P17 TKO), 7531 (P45 KO-replicate 1), and 5886 (P45 KO-replicate 2) were used for downstream analysis.

### Single-Cell RNA-Seq Data Analysis

### Dimensional Reduction, Clustering and Visualization

To integrate the cells from different ages and genotypes, all cells were aligned for each sample and obtained an integrated Seurat object using the Seurat function ‘FindIntegrationAnchors’ and ‘IntegrateData’ with the default parameters, respectively. Using the integrated data, was normalized, log-transformed and scaled the count matrix using the function ‘NormalizeData’ and ‘ScaleData’. The variable genes using the function ‘FindVariableFeatures’(selection.method=“mvp”) were selected and performed dimension reduction analysis with ‘RunPCA’.

To annotate individual cell types in the integrated dataset, all the cells were first clustered using the function ‘FindNeighbors’ and ‘FindClusters’ with a resolution of 0.3 and 1st-30th dimensions. Then the clusters were matched to major cell types using expression of known cell marker genes. As a result, the following 9 major cell types were identified: Alpha1 tanycytes, Alpha2 tanycytes, Beta1 tanycytes, Beta2 tanycytes, Proliferating tanycytes, Astrocytes, Neurons, Ependymal cells and Oligodendrocyte progenitor cells (OPCs). To visualize the integrated data, the 1^{st}-30^{th} dimensions was used to perform nonlinear dimension reduction and obtained UMAP coordinates with the function ‘RunUMAP’.

The molecularly distinct subtypes of TDNs (tanycytes derived neurons) in FIGS. 5A-5J were further characterized. First, the analysis was restricted to cells in the neuron cluster and from the following ages and genotypes: P8 Ctrl, P8 TKO, P17 TKO and P45 TKO. P17 Ctrl and P45 Cntrl were excluded from analysis due to the very small numbers of tanycyte-derived neurons present in these datasets. Second, all the neurons were integrated from different conditions using‘RunHarmony’ in the Harmony R package (71).

Next, the 1^{st}-10^{th} harmony dimensions were used to identify neuronal sub-clusters with a resolution of 0.5. Finally, the clusters were aggregated into individual neuronal subtypes based on known neuron markers and RNA velocity results. To visualize tanycyte-derived neurons, w the 1st-10th harmony dimensions were used to obtain UMAP coordinates with the function ‘RunUMAP’.

### Identification of Differentially Expressed Genes

To identify markers for each cell type and differentially expressed genes (DEGs) between Ctrl and TKO samples, the Seurat function ‘FindAllMarkers’ was used and FindMarkers with the options: min.pct=0.2 or 0.1, log fc.threshold=0.25. Differential genes were retained with an adjusted p-value of <0.001.

### RNA Velocity Analysis

To characterize cellular differentiation trajectories associated with tanycyte-derived neurogenesis, scVelo software (72) was used to perform RNA velocity analysis by comparing levels of spliced and unspliced transcripts. Briefly, bam files were converted for each sample to loom files using a command-line tool (28). These loom files were combined and retained cells which passed filtering in the previous step. Using scVelo, the spliced and unspliced matrix was normalized, filtered the genes and selected the top 1500 variable genes with the function: ‘pp.normalize_per_cell’, ‘pp.filter_genes_dispersion’ and ‘pp. log 1p’. Next, a principal component analysis (PCA) was performed and calculated the velocity vectors and velocity graph using the function: ‘pp.moments’(n_pcs=35, n_neighbors=50), ‘tl.recover_dynamics’, ‘tl.velocity’(mode=‘dynamical’) and ‘tl.velocity_graph’. Finally, the velocities were visualized on the previously calculated UMAP coordinates with the ‘pl.velocity_embedding_grid’ function. The same pipeline was applied to analyze RNA velocity in differentiating tanycyte-derived neurons.

### Cell-Cycle Stage Inference

The function ‘CellCycleScoring’ in the Seurat package was used to calculate cell cycle phase scores (S score and G2/M score), with the G2/M and S phase marker genes obtained from Tirosh et al (73).

### Single-Cell Trajectory Inference

Slingshot (74) was applied to infer differentiation trajectories from alpha2 tanycytes to neurons. To construct the trajectory, the cells in the “Alpha2 tanycytes”, “Proliferating tanycytes” and “Neuron” clusters were included. Slingshot was run using the dimensionality reduction results (UMAP) identified previously. The “Alpha2 tanycytes” cluster was set as the initial cluster to identify lineages with the function “getLineages” and “getCurves” with default parameters. Finally, cells were assigned to the lineages and calculated pseudo-time values for each cell using the function “slingPseudotime.

Monocle 2 (75) was applied to identify developmentally dynamic genes which are significantly altered along the trajectory. First, the expression matrix was converted to Monocle datasets with the function ‘newCellDataSet’, then the Monocle datasets were processed and normalized following the Monocle recommended pipeline, and finally identified DEGs using the “differentialGeneTest” function with the following criteria: q-value<1e-10 and expressed cell number >200.

### Comparison Between Tanycyte-Derived Neurons and Mature Hypothalamic Neurons

To further explore the biological similarity between tanycyte-derived neurons and the broader population of neurons in mouse hypothalamus, the scRNA-seq datasets were first used for mature neurons in hypothalamic arcuate nucleus provided by Campbell, et al (23), and downloaded the cell-by-gene matrix and the annotation file of the mature neuronal cell types from the GEO database under the accession GSE93374. The LIGER (33) package was used to integrate the tanycyte-derived cells identified in the previous rounds of analysis with these mature hypothalamic neurons using the default pipeline recommended in the LIGER guidelines (https://macoskolab.github.io/liger/). After LIGER integration, the integrated datasets were re-clustered and calculated new UMAP coordinates using the function ‘FindNeighbors’, ‘FindClusters’ and ‘RunUMAP’ with the following parameters: reduction=“iNMF”, dims=1:30. Finally, an alluvial plot was made to visualize the alignments between the tanycyte-derived neuronal sub-types (6 subtypes), LIGER clusters (14 clusters) and the mature arcuate neuronal cell types (34 subtypes).

### Single-Cell ATAC-Seg Data Preprocessing

Sequencing output data was processed using the Cell Ranger ATAC software (v.1.0) for alignment, de-duplication, and identification of transposase cut sites. First, the ‘cellranger-atac mkfastq’ function was used for generating fastq files from BCL files. Second, the ‘cellranger-atac count’ function was used to process the fastq files for each library using default parameters and the mouse mm10 reference index provided by 10× Genomics (refdata-cellranger-atac-GRCh38-1.2.0). Finally, the barcoded, aligned, and Tn5-corrected fragment file (fragments.tsv.gz) was obtained for each library and used these for downstream analysis.

### Single-Cell ATAC-Seg Data Analysis

### Generating Union Peaks

The cell-by-peaks matrix were generated for each sample using the same method as described in Satpathy, A. T. et al (68). First, 2.5-kb tiled windows was constructed across the mm10 genome using the local script. Next, a cell-by-window sparse matrix was computed by counting the Tn5 insertion overlaps for each cell, and this matrix was then binarized and inputted to Signac package (0.2.5) to create a Seurat object using ‘CreateSeuratObject.’ Second, the cell-by-window matrix was normalized by TF-IDF methods using ‘RunTFIDF’ and ran a singular value decomposition (SVD) on the TF-IDF normalized matrix with ‘RunSVD.’ The 2^{nd} to 30^{th} dimensions were retained, and identified clusters using SNN graph clustering with ‘FindClusters’ with a resolution of 0.3. Third, to identify high-quality peaks for each cluster in each sample, peaks for each cluster using MACS2 (76) with the command were called: ‘-shift-75—extsize 150—nomodel—callsummits—nolambda—keep-dup all-q 0.05’. The peak summits were then extended to 250 bp on either side to a final width of 500 bp and then filtered by the mm10 v2 blacklist regions (github.com/Boyle-Lab/Blacklist/blob/master/lists/mm10blacklist.v2.bed.gz). The top 50,000 peaks for each cluster in each sample were kept, converted to Granges, and merged into a union peak set with the function ‘reduce’ in the GenomicRanges package. Finally, 107,377 union peaks were obtained, and generated a cell-by-peak sparse matrix for all these cells for downstream analysis.

### Filter Cells by TSS Enrichment, Unique Fragments, and Nucleosome Banding

The TSS enrichment, unique fragments, and nucleosome banding were calculated for each cell using the Signac package. The cell-by-peak sparse matrices were inputted to the ‘CreateSeuratObject’ function to create a Seurat object with default parameters. They were filtered cells using the following criteria: 1) The number of unique nuclear fragments >1000; 2) TSS enrichment score >2; 3) nucleosome banding score <4; 4) blacklist_ratio <0.05. As a result, 8948 (P8 Ctrl) and 13337 (P8 TKO) cells were identified and used for downstream analysis.

### Dimensional Reduction, Clustering and Visualization

The Harmony package was applied to integrate the scATAC-seq data from control and Nfia/b/x TKO samples. First, the Seurat object created in the previous step was put into the Signac process pipeline. A low-dimensional representation of the cell-by-peak matrix using the functions ‘FindTopFeatures’, ‘RunTFIDF’ and ‘RunSVD’ was normalized and obtained. Next, all the cells from both genotypes (control and TKO) using the ‘RunHarmony’ function with the options were integrated: dim.use=2:50, group.by.vars=‘condition’, reduction=‘lsi’ and project.dim=FALSE. Third, the 2^{nd}-30^{th} harmony dimensions were used to identify clusters with a resolution of 0.8, and used the same harmony dimensions to calculate the UMAP coordinates for visualization.

To annotate the cell types for each cluster, the integration method was used in the Seurat package to transfer the previously annotated cell-type labels from scRNA-seq data to scATAC-seq data. First, RNA-seq levels were estimated using the function ‘CreateGeneActivityMatrix’ from the scATAC-seq data using the mm10 genome build gtf file. Next, anchors were found between the scATAC-seq datasets (P8 Ctrl and P8 TKO) and the corresponding scRNA-seq datasets (P8 Ctrl and P8 TKO) using the function ‘transfer.anchors.’ Then with the ‘TransferData’ function, a matrix of cell-type predictions and prediction scores were obtained for each cell in the scATAC-seq dataset. The cells were further filtered with max(prediction score) <0.5. Finally, for each cluster, the number of cells for each predicted cell type was calculated, and set its final annotation based on the cell type that was most highly represented in the cluster.

Using this approach, the following 9 major cell types were identified: Alpha1 tanycytes, Alpha2 tanycytes, Beta1 tanycytes, Beta2 tanycytes, Proliferating tanycytes, Astrocytes, Neurons, Ependymal cells and OPCs.

Global NFI activity and footprint analysis Global NFI activity was inferred with the chromVAR R package (77). The raw cell-by-peak matrix from the total cells was used as input to chromVAR. The mm10 reference genome was used to correct GC bias. he mouse NFI Motifs (including Nfia, Nfib and Nfix) were used in the TransFac2018 database to generate the transcription factor (TF) z-score matrix. The z-score for each cell was used to visualize the global NFI activity using the previously calculated UMAP coordinates.

To analyze the NFI footprint in alpha2 tanycytes, the same methods described in Corces et al. were used (78). First, the NFI motifs and all accessible regions were used to predict the NFI binding sites with the function ‘match motifs’ in the motif matching R package. Second, the Tn5 insertion bias was calculated around every NFI binding site. The aggregated observed 6-bp hexamer table was generated relative to the ±250 bp region from all motif centers, and the aggregated expected 6-bp hexamer table from the mm10 genome was also calculated. The observed/expected (O/E) 6-bp hexamer table was obtained by dividing these two hexamer tables. Finally, the observed Tn5 insertion signal was calculated at ±250 bp from the motif center, and normalized the signal using the O/E 6-bp hexamer table to obtain the final Tn5 bias-corrected signal.

### Differential Peak Analysis

To explore which ATAC regions are changed following Nfia/b/x loss of function, the MAnorm algorithm (79) was applied to perform differential peak analysis between control and Nfia/b/x TKO alpha2 tanycytes. First, cells in the ‘Alpha2 tanycytes’ cluster were selected and then separated these cells by genotype (control and Nfia/b/x TKO). Second, the cells were aggregated of the same genotype by summing the count signals for each peak, then created a new condition-by-peak count matrix, and put it into the MAnorm pipeline. Finally, the MAnorm test was performed and identified differential peaks (4563 peaks enriched in Ctrl and 1333 peaks enriched in TKO) with the cutoff: LOG_P >25, and abs(M_value_rescaled) >0.5.

### De Novo Motif Enrichment Analysis

HOMER software (80) was applied to identify motifs enriched in the differential ATAC regions between control and Nfia/b/x TKO alpha2 tanycytes. The up-regulated peaks and down-regulated peaks were analyzed separately using the Homer function ‘findMotifsGenome.pl’ with the default options except: mm10, -size given, -mask.

### Identification of Genes Directly Regulated by Nfia/b/x

To further explore the biological function of NFI factors in alpha2 tanycytes, a method was developed to infer potential Nfia/b/x targets with the information in scRNA-seq and scATAC-seq data. The methods included the following 3 steps:

### 1. Identification of Nfia/b/x-Binding Regions.

In the previous motif enrichment analysis, it was found that NFI motifs are enriched in the down-regulated peaks (Nfia/b/x TKO/control), so in the first step, it was aimed to identify which down-regulated peaks are bound by NFI factors. Using the NFI motif information in the TRANSFAC2018 database, the NFI motifs in the 4563 down-regulated peaks were scanned with the function ‘matchMotifs’. Next, with the Tn5 insertion signal in P8 Ctrl alpha2 tanycytes, the footprint occupancy score (FOS) were calculated (81) for each predicted NFI binding region, and filtered out the regions with FOS <2. Finally, only the NFI-binding peaks which contain NFI binding sites were kept, and used them for downstream analysis.

2. Identification of Promoters Associated with Nfia/b/x Binding Regions

To identify genes that are potentially regulated by these NFI binding regions, the Cicero algorithm (82) was used to identify all the distal elements-promoter connections genome-wide. First, the cell-by-peak sparse binary matrix was converted into the Cicero pipeline with the functions ‘make_atac_cds’, ‘detectedGenes’ and ‘estimatedSizeFactors’. Next, low-overlapping cell groups were created based on the KNN nearest-neighbors in the UMAP dimension, and aggregated signals for each cell group with the function ‘make_cicero_cds’. The correlation between each peak-peak pair using the function ‘run_cicero’ with default parameters was calculated. Third, the peak pairs were annotated using ‘annotate_cds_by_site’ with mm10 gtf files. The peak pairs with the following criteria were kept: 1) one of the peaks overlapped with ±2 kb of TSS region; and 2) one of the peaks contained at least one NFI binding motif. Finally, NFI-related distal elements-promoter connections from the peak pairs if their co-accessibility score >0.03, or <−0.03 and their distance <150 kb were identified.

3. Inference of Potential Nfia/b/x Targets by Integrating with scRNA-Seq Data.

In this step, the NFI-related distal elements-promoter connections were integrated and differential genes following loss of function of Nfia/b/x to identify NFI target genes. First, enhancer-promoter pairs were selected from the distal elements-promoter connections in Step 2 with co-accessibility scores >0.03. If the gene associated with the promoter in question was down-regulated following loss of function of Nfia/b/x, these genes were treated as potential Nfia/b/x targets.

Conversely, silencer-promoter pairs were selected with co-accessibility scores <−0.03. If the promoter genes were up-regulated following loss of function of Nfia/b/x, these genes were also treated as potential Nfia/b/x targets. Using this approach, 63 NFI target genes were identified.

### GO Term Analysis

To understand the biological functions associated with genes dynamically expressed during the process of alpha2 tanycyte-derived neurogenesis, GOrilla algorithm (83) was applied to identify enriched Gene Ontology terms for each gene cluster using the default parameters (P-value threshold=0.001, ontology=‘Process’). The output of Gene Ontology terms from GOrilla were further processed by REVIGO (84) to remove redundant terms. This pipeline was also used to identify the GO term enrichment in NFI-regulated gene sets.

### Brain Slice Preparation and Cell Type Identification.

To investigate the electrophysiological characteristics of tanycyte-derived and other hypothalamic neurons, acute brain slices were generated as previously described (37). Rax-CreER; Nfia^{lox/lox}; Nfib^{lox/lox}; Nfix^{lox/lox}; CAG-lsl-Sun-GFP mice (P15-P97, male) were anesthetized with isoflurane, decapitated, and the brains were rapidly removed and chilled in ice-cold sucrose solution containing (in mM): 76 NaCl, 25 NaHCO₃, 25 glucose, 75 sucrose, 2.5 KCl, 1.25 NaH₂PO₄, 0.5 CaCl₂, and 7 MgSO₄; pH 7.3. Acute brain slices (300 m) including the hypothalamus were prepared using a vibratome (VT-1200s, Leica) and transferred to warm (32-35° C.) sucrose solution for 30 minutes for recovery. The slices were transferred to warm (32-34° C.) artificial cerebrospinal fluid (aCSF) composed of (in mM): 125 NaCl, 26 NaHCO₃, 2.5 KCl, 1.25 NaH₂PO₄, 1 MgSO₄, 20 glucose, 2 CaCl₂, 0.4 ascorbic acid, 2 pyruvic acid, and 4 L-(+)-lactic acid; pH 7.3, 315 mOsm, and allowed to cool to room temperature (RT). All solutions were continuously bubbled with 95% O₂/5% CO₂. For whole-cell patch-clamp recordings, slices were transferred to a submersion chamber on an upright microscope (Zeiss AxioExaminer, Objective lens: 5×, 0.16 NA and 40×, 1.0 NA) fitted for infrared differential interference contrast (IR-DIC) and fluorescence microscopy. Slices were continuously superfused (2-4 ml/min) with warm, oxygenated aCSF (32-34° C.). Hypothalamic areas and cells were identified under a digital camera (Sensicam QE; Cooke) using either transmitted light or green fluorescence. Tanycytes were identified as GFP-positive cells located in the ependymal cell layer at the 3^{rd} ventricle. Tanycyte-derived cells were identified as GFP-positive cells located in the hypothalamic parenchyma but not in the ependymal cell layer. GFP-negative hypothalamic neurons in the hypothalamic parenchyma, among which were intermingled the sparse tanycyte-derived cells, were targeted as control neurons

### Whole-Cell Recordings and Analysis.

Borosilicate glass pipettes (2-4 MΩ) were filled with an internal solution containing (in mM): 2.7 KCl, 120 KMeSO₄, 9 HEPES, 0.18 EGTA, 4 MgATP, 0.3 NaGTP, 20 phosphocreatine(Na), pH 7.3, 295 mOsm. Biocytin (0.25% weight/volume) was added to the internal solution for post-hoc morphological characterization. Whole-cell patch-clamp recordings were conducted through a Multiclamp 700B amplifier (Molecular Devices) and an ITC-18 (Instrutech) which were controlled by customized routines written in Igor Pro (Wavemetrics). The series resistance averaged 14.2±5.8 MΩ SD (n=81 cells, 12 mice, all <36 MΩ, no significant difference between cell types or age groups, p >0.05, Mann-Whitney U test), and was not compensated. The input resistance was determined by measuring the voltage change in response to a 1 s-long −100 pA hyperpolarizing current step. The current-spike frequency relationship was measured with a series of depolarizing current steps (1 s-long, 0-50 pA, 10 pA increments, 5 s interstimulus intervals). For each current intensity, the total number of action potentials exceeding 0 mV generated during each step was measured and then averaged across the three trials. Spontaneous postsynaptic currents (sPSCs) were measured in voltage-clamp mode at −70 mV. sPSCs were recorded for 25 sec (250 ms-long current traces, 100 times), and ~110 events, on average, were recorded per cell. High amplitude, high frequency depolarizing current steps (10 nA at 100 Hz for 100 ms) were injected into the recorded cells at the end of recording, to increase efficiency of biocytin infusion (85). All signals were low-pass filtered at 10 kHz and sampled at 20 kHz for voltage traces and 100 kHz for series resistance and sPSCs measurements.

### Electrophysiology Data Analysis and Statistical Testing

Data analysis was performed in Igor Pro (WaveMetrics), Excel (Microsoft), ImageJ (NIH), and Minhee analysis (github.com/parkgilbong/Minhee_Analysis_Pack). Data are presented as the mean±SEM unless otherwise noted. A Mann-Whitney U test was used to compare membrane properties and sPSCs frequencies between cell types and between age groups. Spearman's Rho test was used to determine the correlation between sPSC frequency and cell location. The location of the cells (distance to tanycytic layer) was measured from low (5×, 0.16 NA) and high (40×, 1.0 NA) magnification images of the recorded cells using ImageJ. The statistical difference in current-spike frequency relationships was tested by using a two-way ANOVA test with Bonferroni correction. The sPSC events were automatically detected by Mini analysis software with a 10 pA amplitude threshold. In the figures, the statistical significance is expressed as follows: *p<0.05; **p<0.01; or ***p<0.001.

### Visualization of Biocytin-Filled Cells.

Following the electrophysiological experiments, slices were fixed in 4% PFA in 0.01 M PBS at least overnight. After rinsing with PBS, slices were incubated in 0.01 M PBS blocking solution containing 2% Triton X-100 (Sigma-Aldrich) and 5% normal donkey serum (NDS) for 1 h at RT. To visualize biocytin-filled cells, slices were next incubated with a blocking solution containing 1% Triton X-100, 5% NDS, chicken anti-GFP antibody (1:1,000, Aves, Cat. No. GFP-1020), and AlexaFluor 647-conjugated streptavidin overnight on shaker at 4° C. The following day, slices were rinsed with 0.01 M PBS, and incubated with AlexaFluor 488-conjugated donkey-anti-chicken (1:500, Jackson ImmunoResearch, Cat. No. 705-745-155) for 2 h at RT. After rinsing, slices were mounted with Aqua-Poly/Mount (18606-20, Polysciences). A subset of slices was co-stained with mouse anti-NeuN (1:300, Millipore, MAB377) and AlexaFluor 568-conjugated donkey anti-mouse (1:300, ThermoFisher, A10037) antibodies to confirm neuronal identity of the biocytin-filled cells. Fluorescence images were taken with a confocal microscope (LSM 800, Zeiss; 20× objective lens) as z-stack (2 μm-interval) tiled images to cover the extent of the cell's dendritic and axonal processes.

### Small Molecule Modulators

ABC99 (broad-spectrum Wnt activator).
SAG (Smoothened agonist).
LY411575 (gamma secretase inhibitor/Notch antagonist).

The small molecule modulators are blood-brain barrier permeable, and are predicted to stimulate tanycyte-derived neurogenesis. Efficacy in combination (pairwise and three way) in both wildtype and Nfia/b/x-deficient mice.

### AAV Vectors

The following AAV1-based dominant-negative constructs are used to induce neurogenic competence in hypothalamic tanycytes. They are listed by the gene targeted and the overall design of the construct, with the name of the construct in paretheses.

### Sox8:

Sox8 transactivator domain deletion (Kozack_HA_Sox8(DNA binding domain) stop).

Sox8 partial transactivation deletion Kozack_HA_Sox8(C-truncation of transactivation domain)_stop.

Sox8 DNA binding domain fused to KRAB repressor domain (Kozack_HA_Sox9(DNA binding domain)+KRAB_stop).

Sox8 Point Mutations that Disrupt Dimerization or Interaction with Coactivators: Kozack_HA_Sox8(Glu156Asp)_stop.

Sox8 DNA binding domain fused to KRAB repressor domain (Kozack_HA_Sox8(DNA binding domain)+KRAB_stop).

### Sox9:

Sox9 point mutation leads to a C-truncation (Kozack_HA_Sox9(Q412X)_stop).

Sox9 deletion of aa22 to aa234 (Kozack_HA_Sox9(deletion of aa21-234)_stop).

Sox9 deletion of aa27 to aa304 (Kozack_HA_Sox9(deletion of aa27-304)_stop).

Sox9 deletion of aa2 to aa304 (Kozack_HA_Sox9(deletion of aa2-304)_stop).

Sox9 deletion of aa2 to 234+point mutation that affects dimerization (Kozack_HA_Sox9(deletion of aa2-234+Ala76Glu which affects dimerization)_stop).

Sox9 point mutation leads to a C-truncation++point mutation that affects dimerization (Kozack_HA_Sox9(Q412X+Ala76Glu)_stop).

Sox9 transactivator domain deletion (Kozack_HA_Sox9(DNA binding domain)_stop).

### Nfia, Nfib, Nfix:

Nfia transactivator domain deletion (Kozack_HA_NFIAdbd_stop).

Nfib transactivator domain deletion (Kozack_HA_NFIBdbd_stop).

Nfix transactivator domain deletion (Kozack_HA_NFIXdbd_stop).

Nfix MSS_allele_c.1037_1038insT (Kozack_HA NFIX(MSS)_stop).

Nfix transactivation domain truncation exons 1-5 (Kozack_3×HA_NFIX(truncl-5)_stop).

Nfix transactivation domain truncation exons 1-6 (Kozack_3×HA_NFIX(truncl-6)_stop).

Nfix transactivation domain truncation exons 1-7 (Kozack_3×HA_NFIX(truncl-7)_stop).

Nfix transactivation domain truncation exons 1-8 (Kozack_3×HA_NFIX(truncl-8)_stop).

Nfix transactivation domain truncation exons 1-9 (Kozack_3×HA_NFIX(truncl-9)_stop).

Nfix DNA binding domain fused to KRAB repressor domain (NFIXdbd-KRAB) Kozack_3×HA_NFIXdbd_KRAB_stop).

Nfia and Nfib fused DNA binding domains (Kozack_3×HA_NFIXdbd_P2A_V5_NFIAdbd_stop).

Nfia and Nfix fused DNA binding domains (Kozack_3×HA_NFIXdbd_P2A_V5_NFIBdbd_stop).

Nfib and Nfix fused DNA binding domains (Kozack_3×HA_NFIBdbd_P2A_V5_NFIAdbd_stop).

Nfia, Nfib, and Nfix fused DNA binding domains (Kozack_3×HA_NFIXdbd_P2A_FLAG_NFIBdbd_P2A_6×HisTag-NFIAdbd_stop).

### Sequences of Dominant-Negative Constructs

Constructs targeting Sox8:
Name: Kozack_HA_Sox8(DNA binding domain)_stop (SEQ ID NO: 1):
GCCGCCACCATGTATCCCTACGACGTGCCCGATTACGCTTACCCTTACGATGTGCCT
GACTACGCCTATCCATATGACGTGCCAGACTATGCCCTGGACATGAGCGAGGCCCG
CTCCCAGCCGCCCTGCAGCCCGTCCGGCACCGCCAGCTCCATGTCGCACGTGGAGGA
CTCGGACTCGGACGCGCCGCCGTCTCCCGCCGGCTCCGAGGGCCTGGGCCGCGCGG
GGGTCGCGGTGGGGGGCGCCCGGGGCGACCCGGCGGAGGCGGCGGACGAGCGCTT
CCCGGCCTGCATCCGCGACGCCGTGTCGCAGGTGCTCAAGGGCTACGACTGGAGTCT
GGTGCCCATGCCGGTGCGCGGCGGCGGCGGCGGCGCGCTCAAAGCCAAGCCGCATG
TGAAGCGGCCCATGAACGCATTCATGGTGTGGGCGCAGGCGGCGCGCCGCAAGCTG
GCCGACCAGTACCCGCACCTGCACAACGCCGAGCTCAGCAAGACGCTGGGCAAGCT
GTGGCGCTTGCTGAGCGAGAGCGAGAAGCGGCCCTTCGTGGAGGAGGCAGAGCGCC
TTCGCGTGCAGCACAAGAAGGACCACCCCGACTACAAGTGA
Name: Kozack_HA_Sox8(C-truncation of transactivation domain)_stop (SEQ ID NO: 2):
GCCGCCACCATGTATCCCTACGACGTGCCCGATTACGCTTACCCTTACGATGTGCCT
GACTACGCCTATCCATATGACGTGCCAGACTATGCCCTGGACATGAGCGAGGCCCG
CTCCCAGCCGCCCTGCAGCCCGTCCGGCACCGCCAGCTCCATGTCGCACGTGGAGGA
CTCGGACTCGGACGCGCCGCCGTCTCCCGCCGGCTCCGAGGGCCTGGGCCGCGCGG
GGGTCGCGGTGGGGGGCGCCCGGGGCGACCCGGCGGAGGCGGCGGACGAGCGCTT
CCCGGCCTGCATCCGCGACGCCGTGTCGCAGGTGCTCAAGGGCTACGACTGGAGTCT
GGTGCCCATGCCGGTGCGCGGCGGCGGCGGCGGCGCGCTCAAAGCCAAGCCGCATG
TGAAGCGGCCCATGAACGCATTCATGGTGTGGGCGCAGGCGGCGCGCCGCAAGCTG
GCCGACCAGTACCCGCACCTGCACAACGCCGAGCTCAGCAAGACGCTGGGCAAGCT
GTGGCGCTTGCTGAGCGAGAGCGAGAAGCGGCCCTTCGTGGAGGAGGCAGAGCGCC
TTCGCGTGCAGCACAAGAAGGACCACCCCGACTACAAGTACCAGCCACGGCGCAGG
AAGAGCGCCAAAGCCGGCCACAGCGACTCCGACTCGGGCGCGGAGCTGGGACCCCA
CCCTGGCGGCGGTGCCGTGTACAAGGCTGAAGCAGGGCTTGGAGATGGGCACCACC
ATGGCGACCACACAGGGCAGACCCACGGGCCGCCCACCCCGCCCACCACCCCCAAG
ACGGAGCTGCAGCAGGCGGGCGCCAAGCCGGAGCTGAAGCTGGAGGGACGCCGGC
CGGTGGACAGCGGGCGCCAGAACATCGACTTCAGCAACGTGGACATCTCGGAGCTC
AGCAGCGAGGTCATGGGCACCATGGACGCCTTCGACGTCCACGAGTTCGACCAGTA
CCTGCCCCTGGGCGGCCCCGCCCCACCCGAGTGA
Name: Kozack_HA_Sox8(Glu156Asp)_stop (SEQ ID NO: 3):
GCCGCCACCATGTATCCCTACGACGTGCCCGATTACGCTTACCCTTACGATGTGCCT
GACTACGCCTATCCATATGACGTGCCAGACTATGCCCTGGACATGAGCGAGGCCCG
CTCCCAGCCGCCCTGCAGCCCGTCCGGCACCGCCAGCTCCATGTCGCACGTGGAGGA
CTCGGACTCGGACGCGCCGCCGTCTCCCGCCGGCTCCGAGGGCCTGGGCCGCGCGG
GGGTCGCGGTGGGGGGCGCCCGGGGCGACCCGGCGGAGGCGGCGGACGAGCGCTT
CCCGGCCTGCATCCGCGACGCCGTGTCGCAGGTGCTCAAGGGCTACGACTGGAGTCT
GGTGCCCATGCCGGTGCGCGGCGGCGGCGGCGGCGCGCTCAAAGCCAAGCCGCATG
TGAAGCGGCCCATGAACGCATTCATGGTGTGGGCGCAGGCGGCGCGCCGCAAGCTG
GCCGACCAGTACCCGCACCTGCACAACGCCGAGCTCAGCAAGACGCTGGGCAAGCT
GTGGCGCTTGCTGAGCGAGAGCGAGAAGCGGCCCTTCGTGGAGGAGGCAGACCGCC
TTCGCGTGCAGCACAAGAAGGACCACCCCGACTACAAGTACCAGCCACGGCGCAGG
AAGAGCGCCAAAGCCGGCCACAGCGACTCCGACTCGGGCGCGGAGCTGGGACCCCA
CCCTGGCGGCGGTGCCGTGTACAAGGCTGAAGCAGGGCTTGGAGATGGGCACCACC
ATGGCGACCACACAGGGCAGACCCACGGGCCGCCCACCCCGCCCACCACCCCCAAG
ACGGAGCTGCAGCAGGCGGGCGCCAAGCCGGAGCTGAAGCTGGAGGGACGCCGGC
CGGTGGACAGCGGGCGCCAGAACATCGACTTCAGCAACGTGGACATCTCGGAGCTC
AGCAGCGAGGTCATGGGCACCATGGACGCCTTCGACGTCCACGAGTTCGACCAGTA
CCTGCCCCTGGGCGGCCCCGCCCCACCCGAGCCGGGCCAGGCCTATGGGGGCGCCT
ACTTCCACGCCGGGGCGTCCCCCGTGTGGGCCCACAAGAGTGCCCCGTCGGCCTCCG
CGTCGCCCACCGAGACGGGTCCCCCACGGCCGCACATCAAGACGGAGCAGCCGAGC
CCCGGCCACTACGGCGACCAGCCCCGAGGCTCGCCCGACTACGGTTCCTGCAGCGG
CCAGTCCAGCGCCACCCCGGCCGCCCCCGCCGGCCCCTTCGCCGGCTCACAGGGCG
ACTATGGCGACCTGCAGGCCTCCAGCTACTATGGTGCCTACCCTGGCTACGCACCCG
GCCTCTACCAGTACCCCTGCTTCCACTCGCCGCGCCGGCCCTACGCCTCACCCCTGCT
CAACGGCCTGGCCCTGCCGCCCGCCCACAGCCCCACCAGTCACTGGGACCAGCCGG
TGTACACCACCCTGACCAGGCCCTGA
Name: Kozack_HA_Sox8(DNA binding domain) + KRAB_stop (SEQ ID NO: 4):
GCCGCCACCATGTATCCCTACGACGTGCCCGATTACGCTTACCCTTACGATGTGCCT
GACTACGCCTATCCATATGACGTGCCAGACTATGCCCTGGACATGAGCGAGGCCCG
CTCCCAGCCGCCCTGCAGCCCGTCCGGCACCGCCAGCTCCATGTCGCACGTGGAGGA
CTCGGACTCGGACGCGCCGCCGTCTCCCGCCGGCTCCGAGGGCCTGGGCCGCGCGG
GGGTCGCGGTGGGGGGCGCCCGGGGCGACCCGGCGGAGGCGGCGGACGAGCGCTT
CCCGGCCTGCATCCGCGACGCCGTGTCGCAGGTGCTCAAGGGCTACGACTGGAGTCT
GGTGCCCATGCCGGTGCGCGGCGGCGGCGGCGGCGCGCTCAAAGCCAAGCCGCATG
TGAAGCGGCCCATGAACGCATTCATGGTGTGGGCGCAGGCGGCGCGCCGCAAGCTG
GCCGACCAGTACCCGCACCTGCACAACGCCGAGCTCAGCAAGACGCTGGGCAAGCT
GTGGCGCTTGCTGAGCGAGAGCGAGAAGCGGCCCTTCGTGGAGGAGGCAGAGCGCC
TTCGCGTGCAGCACAAGAAGGACCACCCCGACTACAAGGATGCCAAGAGCCTGACC
GCCTGGTCTAGAACCCTGGTCACCTTCAAGGACGTGTTCGTGGACTTCACCCGGGAA
GAGTGGAAGCTGCTGGATACAGCCCAGCAGATCGTGTACCGGAACGTGATGCTGGA
AAACTACAAGAACCTGGTGTCCCTGGGCTACCAGCTGACCAAGCCTGACGTGATCCT
GCGGCTGGAAAAGGGCGAAGAACCTTGGCTGGTGTGA
Constructs targeting Sox9:
Name: Kozack_HA_Sox9(Q412X)_stop (SEQ ID NO: 5):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGCGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGGAGGCCACGGAG
CAGACGCACATCTCCCCCAACGCCATCTTCAAGGCGCTGCAGGCCGACTCGCCACAC
TCCTCCTCCGGCATGAGCGAGGTGCACTCCCCCGGCGAGCACTCGGGGCAATCCCA
GGGCCCACCGACCCCACCCACCACCCCCAAAACCGACGTGCAGCCGGGCAAGGCTG
ACCTGAAGCGAGAGGGGCGCCCCTTGCCAGAGGGGGGCAGACAGCCCCCTATCGAC
TTCCGCGACGTGGACATCGGCGAGCTGAGCAGCGACGTCATCTCCAACATCGAGAC
CTTCGATGTCAACGAGTTTGACCAGTACCTGCCGCCCAACGGCCACCCGGGGGTGCC
GGCCACGCACGGCCAGGTCACCTACACGGGCAGCTACGGCATCAGCAGCACCGCGG
CCACCCCGGCGAGCGCGGGCCACGTGTGGATGTCCAAGCAGCAGGCGCCGCCGCCA
CCCCCGCAGCAGCCCCCACAGGCCCCGCCGGCCCCGCAGGCGCCCCCGCAGCCGCA
GGCGGCGCCCCCACAGCAGCCGGCGGCACCCCCGCAGCAGCCACAGGCGCACACGC
TGACCACGCTGAGCAGCGAGCCGGGCCAGTCCCAGCGAACGCACATCAAGACGGAG
CAGCTGAGCCCCAGCCACTACAGCGAGCAGCAGTAG
Name: Kozack_HA_Sox9(deletion of aa21-234)_stop (SEQ ID NO: 6):
GCCGCCACCATGTATCCCTACGACGTGCCCGATTACGCTTACCCTTACGATGTGCCT
GACTACGCCTATCCATATGACGTGCCAGACTATGCCGCCCCCAGCCCCACCATGTCC
GAGGACTCCGCGGGCTCGCCCTGCCCGTCGGGCTCCGGCTCGGACACCGAGAACAC
GCGGCCCCAGGAGAACACGTTCCCCAAGGGCGAGCCCGATCTGAAGAAGGAGAGC
GAGGAGGACAAGTTCCCCGTGTGCATCCGCGAGGCGGTCAGCCAGGTGCTCAAAGG
CTACGACTGGACGCTGGTGCCCATGCCGGTGCGCGTCAACGGCTCCAGCAAGAACA
AGCCGCACGTCAAGCGGCCCATGAACGCCTTCATGGTGTGGGCGCAGGCGGCGCGC
AGGAAGCTCGCGGACCAGTACCCGCACTTGCACAACGCCGAGCTCAGCAAGACGCT
GGGCAAGCTCTGGAGACTTCTGAACGAGAGCGAGAAGCGGCCCTTCGTGGAGGAGG
CGGAGCGGCTGCGCGTGCAGCACAAGAAGGACCACCCGGATTACAAGTACCAGCCG
CGGCGGAGGAAGTCGGTGAAGAACGGGCAGGCGGAGGCAGAGGAGGCCACGGAGC
AGACGCACATCTCCCCCAACGCCATCTTCAAGGCGCTGCAGGCCGACTCGCCACACT
CCTCCTCCGGCATGAGCGAGGTGCACTCCCCCGGCGAGCACTCGGGGCAATCCCAG
GGCCCATAG
Name: Kozack_HA_Sox9(deletion of aa27-304)_stop (SEQ ID NO: 7):
Sequence:GCCGCCACCATGTATCCCTACGACGTGCCCGATTACGCTTACCCTTACGAT
GTGCCTGACTACGCCTATCCATATGACGTGCCAGACTATGCCTCCGAGGACTCCGCG
GGCTCGCCCTGCCCGTCGGGCTCCGGCTCGGACACCGAGAACACGCGGCCCCAGGA
GAACACGTTCCCCAAGGGCGAGCCCGATCTGAAGAAGGAGAGCGAGGAGGACAAG
TTCCCCGTGTGCATCCGCGAGGCGGTCAGCCAGGTGCTCAAAGGCTACGACTGGAC
GCTGGTGCCCATGCCGGTGCGCGTCAACGGCTCCAGCAAGAACAAGCCGCACGTCA
AGCGGCCCATGAACGCCTTCATGGTGTGGGCGCAGGCGGCGCGCAGGAAGCTCGCG
GACCAGTACCCGCACTTGCACAACGCCGAGCTCAGCAAGACGCTGGGCAAGCTCTG
GAGACTTCTGAACGAGAGCGAGAAGCGGCCCTTCGTGGAGGAGGCGGAGCGGCTGC
GCGTGCAGCACAAGAAGGACCACCCGGATTACAAGTACCAGCCGCGGCGGAGGAA
GTCGGTGAAGAACGGGCAGGCGGAGGCAGAGGAGGCCACGGAGCAGACGCACATC
TCCCCCAACGCCATCTTCAAGGCGCTGCAGGCCGACTCGCCACACTCCTCCTCCGGC
ATGAGCGAGGTGCACTCCCCCGGCGAGCACTCGGGGCAATCCCAGGGCCCACCGAC
CCCACCCACCACCCCCAAAACCGACGTGCAGCCGGGCAAGGCTGACCTGAAGCGAG
AGGGGCGCCCCTTGCCAGAGGGGGGCAGACAGCCCCCTATCGACTTCCGCGACGTG
GACATCGGCGAGCTGAGCAGCGACGTCATCTCCAACATCGAGACCTTCGATGTCAA
CGAGTTTGACCAGTACCTGCCGCCCAACGGCCACCCGTAG
Name: Kozack_HA_Sox9(deletion of aa2-304)_stop (SEQ ID NO: 8):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGCGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGGAGGCCACGGAG
CAGACGCACATCTCCCCCAACGCCATCTTCAAGGCGCTGCAGGCCGACTCGCCACAC
TCCTCCTCCGGCATGAGCGAGGTGCACTCCCCCGGCGAGCACTCGGGGCAATCCCA
GGGCCCATAG
Name: Kozack_HA_Sox9(deletion of aa2-234 + Ala76Glu wich affects dimerization)_stop
(SEQ ID NO: 9):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGAGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGGAGGCCACGGAG
CAGACGCACATCTCCCCCAACGCCATCTTCAAGGCGCTGCAGGCCGACTCGCCACAC
TCCTCCTCCGGCATGAGCGAGGTGCACTCCCCCGGCGAGCACTCGGGGCAATCCCA
GGGCCCATAG
Name: Kozack_HA_Sox9(Q412X + Ala76Glu)_stop (SEQ ID NO: 10):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGAGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGGAGGCCACGGAG
CAGACGCACATCTCCCCCAACGCCATCTTCAAGGCGCTGCAGGCCGACTCGCCACAC
TCCTCCTCCGGCATGAGCGAGGTGCACTCCCCCGGCGAGCACTCGGGGCAATCCCA
GGGCCCACCGACCCCACCCACCACCCCCAAAACCGACGTGCAGCCGGGCAAGGCTG
ACCTGAAGCGAGAGGGGCGCCCCTTGCCAGAGGGGGGCAGACAGCCCCCTATCGAC
TTCCGCGACGTGGACATCGGCGAGCTGAGCAGCGACGTCATCTCCAACATCGAGAC
CTTCGATGTCAACGAGTTTGACCAGTACCTGCCGCCCAACGGCCACCCGGGGGTGCC
GGCCACGCACGGCCAGGTCACCTACACGGGCAGCTACGGCATCAGCAGCACCGCGG
CCACCCCGGCGAGCGCGGGCCACGTGTGGATGTCCAAGCAGCAGGCGCCGCCGCCA
CCCCCGCAGCAGCCCCCACAGGCCCCGCCGGCCCCGCAGGCGCCCCCGCAGCCGCA
GGCGGCGCCCCCACAGCAGCCGGCGGCACCCCCGCAGCAGCCACAGGCGCACACGC
TGACCACGCTGAGCAGCGAGCCGGGCCAGTCCCAGCGAACGCACATCAAGACGGAG
CAGCTGAGCCCCAGCCACTACAGCGAGCAGCAGTAG
Name: Kozack_HA_Sox9(DNA binding domain) + KRAB_stop (SEQ ID NO: 11):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGCGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGGATGCCAAGAGC
CTGACCGCCTGGTCTAGAACCCTGGTCACCTTCAAGGACGTGTTCGTGGACTTCACC
CGGGAAGAGTGGAAGCTGCTGGATACAGCCCAGCAGATCGTGTACCGGAACGTGAT
GCTGGAAAACTACAAGAACCTGGTGTCCCTGGGCTACCAGCTGACCAAGCCTGACG
TGATCCTGCGGCTGGAAAAGGGCGAAGAACCTTGGCTGGTGTGA
Name: Kozack_HA_Sox9(DNA binding domain)_stop (SEQ ID NO: 12):
GCCGCCACCATGTATCCCTACGACGTGCCCGATTACGCTTACCCTTACGATGTGCCT
GACTACGCCTATCCATATGACGTGCCAGACTATGCCAATCTCCTGGACCCCTTCATG
AAGATGACCGACGAGCAGGAGAAGGGCCTGTCCGGCGCCCCCAGCCCCACCATGTC
CGAGGACTCCGCGGGCTCGCCCTGCCCGTCGGGCTCCGGCTCGGACACCGAGAACA
CGCGGCCCCAGGAGAACACGTTCCCCAAGGGCGAGCCCGATCTGAAGAAGGAGAG
CGAGGAGGACAAGTTCCCCGTGTGCATCCGCGAGGCGGTCAGCCAGGTGCTCAAAG
GCTACGACTGGACGCTGGTGCCCATGCCGGTGCGCGTCAACGGCTCCAGCAAGAAC
AAGCCGCACGTCAAGCGGCCCATGAACGCCTTCATGGTGTGGGCGCAGGCGGCGCG
CAGGAAGCTCGCGGACCAGTACCCGCACTTGCACAACGCCGAGCTCAGCAAGACGC
TGGGCAAGCTCTGGAGACTTCTGAACGAGAGCGAGAAGCGGCCCTTCGTGGAGGAG
GCGGAGCGGCTGCGCGTGCAGCACAAGAAGGACCACCCGGATTACAAGTACCAGCC
GCGGCGGAGGAAGTCGGTGAAGAACGGGCAGGCGGAGGCAGAGTGA
Constructs targeting Nfia:
Name: Kozack_HA_NFIAdbd_stop (SEQ ID NO: 13):
GCCGCCACCATGTATCCCTACGACGTGCCCGATTACGCTTACCCTTACGATGTGCCT
GACTACGCCTATCCATATGACGTGCCAGACTATGCCTATTCTCCGCTCTGTCTCACCC
AGGATGAATTTCATCCTTTCATCGAAGCACTTCTGCCCCACGTCCGAGCCTTTGCCTA
CACATGGTTCAACCTGCAGGCCCGAAAACGAAAATACTTCAAAAAACATGAAAAGC
GTATGTCAAAAGAAGAAGAGAGAGCCGTGAAGGATGAATTGCTAAGTGAAAAACC
AGAGGTCAAGCAGAAGTGGGCATCTCGACTTCTGGCAAAGTTGCGGAAAGATATCC
GACCCGAATATCGAGAGGATTTTGTTCTTACAGTTACAGGGAAAAAACCTCCATGTT
GTGTTCTTTCCAACCCAGACCAGAAAGGCAAGATGCGAAGAATTGACTGCCTCCGC
CAGGCAGATAAAGTCTGGAGGTTGGACCTTGTTATGGTGATTTTGTTTAAAGGTATT
CCGCTGGAAAGTACTGATGGCGAGCGCCTTGTAAAGTCCCCACAATGCTCTAATCCA
GGGCTCTGTGTCCAACCCCATCACATAGGGGTTTCTGTTAAGGAACTCGATTTATAT
TTGGCATACTTTGTGCATGCAGCAGATTCAAGTCAATCTGAAAGTCCCAGCCAGCCA
AGTGACGCTGACATTAAGGACCAGCCAGAAAATTAA
Constructs targeting Nfib:
Name: Kozack_HA_NFIBdbd_stop (SEQ ID NO: 14):
GCCGCCACCATGTATCCCTACGACGTGCCCGATTACGCTTACCCTTACGATGTGCCT
GACTACGCCTATCCATATGACGTGCCAGACTATGCCATGTATTCTCCCATCTGTCTCA
CTCAGGATGAATTTCACCCATTCATCGAGGCACTTCTTCCACATGTCCGTGCAATTGC
CTATACTTGGTTCAACCTGCAGGCTCGAAAACGCAAGTACTTTAAAAAGCATGAGA
AGCGAATGTCAAAGGATGAAGAAAGAGCAGTCAAAGATGAGCTTCTCAGTGAAAA
GCCTGAAATCAAACAGAAGTGGGCATCCAGGCTCCTTGCCAAACTGCGCAAAGATA
TTCGCCAGGAGTATCGAGAGGACTTTGTGCTCACCGTGACTGGCAAGAAGCACCCGT
GCTGTGTCTTATCCAATCCCGACCAGAAGGGTAAGATTAGGAGAATCGACTGCCTGC
GACAGGCAGACAAAGTCTGGCGTCTGGATCTAGTCATGGTGATCCTGTTCAAAGGC
ATCCCCTTGGAAAGTACCGATGGAGAGCGGCTCATGAAATCCCCACATTGCACAAA
CCCAGCACTTTGTGTCCAGCCACATCATATCACAGTATCAGTTAAGGAGCTTGATTT
GTTTTTGGCATACTACGTGCAGGAGCAAGATTCTGGACAATCAGGAAGTCCAAGCC
ACAATGATCCTGCCAAGAATCCTCCATAA
Constructs targeting Nfix:
Name: Kozack_HA_NFIXdbd_stop (SEQ ID NO: 15):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACTAA
Name: Kozack_HA_NFIX(MSS)_stop (SEQ ID NO: 16):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCAGCACCACCAAGCGCCCCAAGTCCATCGATGACAGTGAGATGGAGAGCCCTGTT
GATGACGTGTTCTATCCCGGGACAGGCCGTTCCCCAGCAGCTGGCAGCAGCCAGTCC
AGCGGGTGGCCCAACGATGTGGATGCAGGCCCGGCTTCTCTAAAGAAGTCAGGAAA
GCTGGACTTCTGCAGTGCCCTCTCCTCTCAGGGCAGCTCCCCGCGCATGGCTTTTCAC
CCACCACCCGCTGCCTGTGCTTGCTGGAGTCAGACCAGGGAGCCCCCGGGCCACAG
CATCAGCCCTGCACTTCCCCTCCACGTCCATCATCCAGCAGTCGAGCCCGTATTTCA
CGCACCCGACCATCCGCTACCACCACCACCACGGGCAGGACTCACTGAAGGAGTTT
GTGCAGTTTGTGTGCTCGGATGGCTCGGGCCAGGCCACCGGACAGCCCAACGGTAG
Name: Kozack_3xHA_NFIX(trunc1-5)_stop (SEQ ID NO: 17):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCTAA
Name: Kozack_3xHA_NFIX(trunc1-6)_stop (SEQ ID NO: 18):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCAGCACCACCAAGCGCCCCAAGTCCATCGATGACAGTGAGATGGAGAGCCCTGTT
GATGACGTGTTCTATCCCGGGACAGGCCGTTCCCCAGCAGCTGGCAGCAGCCAGTCC
AGCGGGTGGCCCAACGATGTGGATGCATAA
Name: Kozack_3xHA_NFIX(trunc1-7)_stop (SEQ ID NO: 19):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCAGCACCACCAAGCGCCCCAAGTCCATCGATGACAGTGAGATGGAGAGCCCTGTT
GATGACGTGTTCTATCCCGGGACAGGCCGTTCCCCAGCAGCTGGCAGCAGCCAGTCC
AGCGGGTGGCCCAACGATGTGGATGCAGGCCCGGCTTCTCTAAAGAAGTCAGGAAA
GCTGGACTTCTGCAGTGCCCTCTCCTCTCAGGGCAGCTCCCCGCGCATGGCTTTCAC
CCACCACCCGCTGCCTGTGCTTGCTGGAGTCAGACCATAA
Name: Kozack_3xHA_NFIX(trunc1-8)_stop (SEQ ID NO: 20):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCAGCACCACCAAGCGCCCCAAGTCCATCGATGACAGTGAGATGGAGAGCCCTGTT
GATGACGTGTTCTATCCCGGGACAGGCCGTTCCCCAGCAGCTGGCAGCAGCCAGTCC
AGCGGGTGGCCCAACGATGTGGATGCAGGCCCGGCTTCTCTAAAGAAGTCAGGAAA
GCTGGACTTCTGCAGTGCCCTCTCCTCTCAGGGCAGCTCCCCGCGCATGGCTTTCAC
CCACCACCCGCTGCCTGTGCTTGCTGGAGTCAGACCAGGGAGCCCCCGGGCCACAG
CATCAGCCCTGCACTTCCCCTCCACGTCCATCATCCAGCAGTCGAGCCCGTATTTCA
CGCACCCGACCATCCGCTACCACCACCACCACGGGCAGGACTCACTGAAGGAGTTT
GTGCAGTTTGTGTGCTCGGATGGCTCGGGCCAGGCCACCGGACAGTAA
Name: Kozack_3xHA_NFIX(trunc1-9)_stop (SEQ ID NO: 21):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGACT
GTTTTGTGACTTCCGGGGTCTGGAATGTGACGGAGCTGGTGAGAGTATCACAGACTC
CTGTTGCAACAGCATCAGGGCCCAACTTCTCCCTGGCGGACCTGGAGAGTCCCAGCT
ACTACAACATCAACCAGGTGACCCTGGGGCGGCGGTCCATCACCTCCCCTCCTTCCA
CCAGCACCACCAAGCGCCCCAAGTCCATCGATGACAGTGAGATGGAGAGCCCTGTT
GATGACGTGTTCTATCCCGGGACAGGCCGTTCCCCAGCAGCTGGCAGCAGCCAGTCC
AGCGGGTGGCCCAACGATGTGGATGCAGGCCCGGCTTCTCTAAAGAAGTCAGGAAA
GCTGGACTTCTGCAGTGCCCTCTCCTCTCAGGGCAGCTCCCCGCGCATGGCTTTCAC
CCACCACCCGCTGCCTGTGCTTGCTGGAGTCAGACCAGGGAGCCCCCGGGCCACAG
CATCAGCCCTGCACTTCCCCTCCACGTCCATCATCCAGCAGTCGAGCCCGTATTTCA
CGCACCCGACCATCCGCTACCACCACCACCACGGGCAGGACTCACTGAAGGAGTTT
GTGCAGTTTGTGTGCTCGGATGGCTCGGGCCAGGCCACCGGACAGCCCAACGGTAG
CGGCCAGGGCAAAGTCCCGGGGTCATTTTTGCTACCGCCGCCGCCTCCAGTGGCCAG
ACCTGTGCCCCTTCCTATGCCTGATTCCAAATCCACCAGCACTGCCCCAGACGGCGC
CGCCTTGACTCCTCCATCACCTTAA
Name: Kozack_3xHA_NFIXdbd KRAB_stop (SEQ ID NO: 22):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACGATGCCAAGAGCCTGACCGCCT
GGTCTAGAACCCTGGTCACCTTCAAGGACGTGTTCGTGGACTTCACCCGGGAAGAGT
GGAAGCTGCTGGATACAGCCCAGCAGATCGTGTACCGGAACGTGATGCTGGAAAAC
TACAAGAACCTGGTGTCCCTGGGCTACCAGCTGACCAAGCCTGACGTGATCCTGCGG
CTGGAAAAGGGCGAAGAACCTTGGCTGGTGTAA
Constructs targeting Nfia and Nfib:
Name: Kozack_3xHA_NFIBdbd_P2A_V5_NFIAdbd_stop (SEQ ID NO: 23):
GCCGCCACCATGTATCCCTACGACGTGCCCGATTACGCTTACCCTTACGATGTGCCT
GACTACGCCTATCCATATGACGTGCCAGACTATGCCATGTATTCTCCCATCTGTCTCA
CTCAGGATGAATTTCACCCATTCATCGAGGCACTTCTTCCACATGTCCGTGCAATTGC
CTATACTTGGTTCAACCTGCAGGCTCGAAAACGCAAGTACTTTAAAAAGCATGAGA
AGCGAATGTCAAAGGATGAAGAAAGAGCAGTCAAAGATGAGCTTCTCAGTGAAAA
GCCTGAAATCAAACAGAAGTGGGCATCCAGGCTCCTTGCCAAACTGCGCAAAGATA
TTCGCCAGGAGTATCGAGAGGACTTTGTGCTCACCGTGACTGGCAAGAAGCACCCGT
GCTGTGTCTTATCCAATCCCGACCAGAAGGGTAAGATTAGGAGAATCGACTGCCTGC
GACAGGCAGACAAAGTCTGGCGTCTGGATCTAGTCATGGTGATCCTGTTCAAAGGC
ATCCCCTTGGAAAGTACCGATGGAGAGCGGCTCATGAAATCCCCACATTGCACAAA
CCCAGCACTTTGTGTCCAGCCACATCATATCACAGTATCAGTTAAGGAGCTTGATTT
GTTTTTGGCATACTACGTGCAGGAGCAAGATTCTGGACAATCAGGAAGTCCAAGCC
ACAATGATCCTGCCAAGAATCCTCCAaagcttggaagcggagctactaacttcagcctgctgaagcaggctggag
acgtggaggagaaccctggacctatggagagcgacgagagcggcctgggtaagcctatccctaaccctctcctcggtctcgattcta
cgTATTCTCCGCTCTGTCTCACCCAGGATGAATTTCATCCTTTCATCGAAGCACTTCTGCC
CCACGTCCGAGCCTTTGCCTACACATGGTTCAACCTGCAGGCCCGAAAACGAAAAT
ACTTCAAAAAACATGAAAAGCGTATGTCAAAAGAAGAAGAGAGAGCCGTGAAGGA
TGAATTGCTAAGTGAAAAACCAGAGGTCAAGCAGAAGTGGGCATCTCGACTTCTGG
CAAAGTTGCGGAAAGATATCCGACCCGAATATCGAGAGGATTTTGTTCTTACAGTTA
CAGGGAAAAAACCTCCATGTTGTGTTCTTTCCAACCCAGACCAGAAAGGCAAGATG
CGAAGAATTGACTGCCTCCGCCAGGCAGATAAAGTCTGGAGGTTGGACCTTGTTATG
GTGATTTTGTTTAAAGGTATTCCGCTGGAAAGTACTGATGGCGAGCGCCTTGTAAAG
TCCCCACAATGCTCTAATCCAGGGCTCTGTGTCCAACCCCATCACATAGGGGTTTCT
GTTAAGGAACTCGATTTATATTTGGCATACTTTGTGCATGCAGCAGATTCAAGTCAA
TCTGAAAGTCCCAGCCAGCCAAGTGACGCTGACATTAAGGACCAGCCAGAAAATTA
A
Constructs targeting Nfia and Nfix:
Name: Kozack_3xHA_NFIXdbd_P2A_V5_NFIBdbd_stop (SEQ ID NO: 24):
GCCGCCACCATGTATCCCTACGACGTGCCCGATTACGCTTACCCTTACGATGTGCCT
GACTACGCCTATCCATATGACGTGCCAGACTATGCCATGTATTCTCCCATCTGTCTCA
CTCAGGATGAATTTCACCCATTCATCGAGGCACTTCTTCCACATGTCCGTGCAATTGC
CTATACTTGGTTCAACCTGCAGGCTCGAAAACGCAAGTACTTTAAAAAGCATGAGA
AGCGAATGTCAAAGGATGAAGAAAGAGCAGTCAAAGATGAGCTTCTCAGTGAAAA
GCCTGAAATCAAACAGAAGTGGGCATCCAGGCTCCTTGCCAAACTGCGCAAAGATA
TTCGCCAGGAGTATCGAGAGGACTTTGTGCTCACCGTGACTGGCAAGAAGCACCCGT
GCTGTGTCTTATCCAATCCCGACCAGAAGGGTAAGATTAGGAGAATCGACTGCCTGC
GACAGGCAGACAAAGTCTGGCGTCTGGATCTAGTCATGGTGATCCTGTTCAAAGGC
ATCCCCTTGGAAAGTACCGATGGAGAGCGGCTCATGAAATCCCCACATTGCACAAA
CCCAGCACTTTGTGTCCAGCCACATCATATCACAGTATCAGTTAAGGAGCTTGATTT
GTTTTTGGCATACTACGTGCAGGAGCAAGATTCTGGACAATCAGGAAGTCCAAGCC
ACAATGATCCTGCCAAGAATCCTCCAaagcttggaagcggagctactaacttcagcctgctgaagcaggctggag
acgtggaggagaaccctggacctatggagagcgacgagagcggcctgggtaagcctatccctaaccctctcctcggtctcgattcta
cgTATTCTCCGCTCTGTCTCACCCAGGATGAATTTCATCCTTTCATCGAAGCACTTCTGCC
CCACGTCCGAGCCTTTGCCTACACATGGTTCAACCTGCAGGCCCGAAAACGAAAAT
ACTTCAAAAAACATGAAAAGCGTATGTCAAAAGAAGAAGAGAGAGCCGTGAAGGA
TGAATTGCTAAGTGAAAAACCAGAGGTCAAGCAGAAGTGGGCATCTCGACTTCTGG
CAAAGTTGCGGAAAGATATCCGACCCGAATATCGAGAGGATTTTGTTCTTACAGTTA
CAGGGAAAAAACCTCCATGTTGTGTTCTTTCCAACCCAGACCAGAAAGGCAAGATG
CGAAGAATTGACTGCCTCCGCCAGGCAGATAAAGTCTGGAGGTTGGACCTTGTTATG
GTGATTTTGTTTAAAGGTATTCCGCTGGAAAGTACTGATGGCGAGCGCCTTGTAAAG
TCCCCACAATGCTCTAATCCAGGGCTCTGTGTCCAACCCCATCACATAGGGGTTTCT
GTTAAGGAACTCGATTTATATTTGGCATACTTTGTGCATGCAGCAGATTCAAGTCAA
TCTGAAAGTCCCAGCCAGCCAAGTGACGCTGACATTAAGGACCAGCCAGAAAATTA
A
Constructs targeting Nfib and Nfix:
Name: Kozack_3xHA_NFIXdbd_P2A_V5_NFIBdbd_stop (SEQ ID NO: 25):
GCCGCCACCATGTATCCCTACGACGTGCCCGATTACGCTTACCCTTACGATGTGCCT
GACTACGCCTATCCATATGACGTGCCAGACTATGCCATGTATTCTCCCATCTGTCTCA
CTCAGGATGAATTTCACCCATTCATCGAGGCACTTCTTCCACATGTCCGTGCAATTGC
CTATACTTGGTTCAACCTGCAGGCTCGAAAACGCAAGTACTTTAAAAAGCATGAGA
AGCGAATGTCAAAGGATGAAGAAAGAGCAGTCAAAGATGAGCTTCTCAGTGAAAA
GCCTGAAATCAAACAGAAGTGGGCATCCAGGCTCCTTGCCAAACTGCGCAAAGATA
TTCGCCAGGAGTATCGAGAGGACTTTGTGCTCACCGTGACTGGCAAGAAGCACCCGT
GCTGTGTCTTATCCAATCCCGACCAGAAGGGTAAGATTAGGAGAATCGACTGCCTGC
GACAGGCAGACAAAGTCTGGCGTCTGGATCTAGTCATGGTGATCCTGTTCAAAGGC
ATCCCCTTGGAAAGTACCGATGGAGAGCGGCTCATGAAATCCCCACATTGCACAAA
CCCAGCACTTTGTGTCCAGCCACATCATATCACAGTATCAGTTAAGGAGCTTGATTT
GTTTTTGGCATACTACGTGCAGGAGCAAGATTCTGGACAATCAGGAAGTCCAAGCC
ACAATGATCCTGCCAAGAATCCTCCAaagcttggaagcggagctactaacttcagcctgctgaagcaggctggag
acgtggaggagaaccctggacctatggagagcgacgagagcggcctgggtaagcctatccctaaccctctcctcggtctcgattcta
cgTATTCTCCGCTCTGTCTCACCCAGGATGAATTTCATCCTTTCATCGAAGCACTTCTGCC
CCACGTCCGAGCCTTTGCCTACACATGGTTCAACCTGCAGGCCCGAAAACGAAAAT
ACTTCAAAAAACATGAAAAGCGTATGTCAAAAGAAGAAGAGAGAGCCGTGAAGGA
TGAATTGCTAAGTGAAAAACCAGAGGTCAAGCAGAAGTGGGCATCTCGACTTCTGG
CAAAGTTGCGGAAAGATATCCGACCCGAATATCGAGAGGATTTTGTTCTTACAGTTA
CAGGGAAAAAACCTCCATGTTGTGTTCTTTCCAACCCAGACCAGAAAGGCAAGATG
CGAAGAATTGACTGCCTCCGCCAGGCAGATAAAGTCTGGAGGTTGGACCTTGTTATG
GTGATTTTGTTTAAAGGTATTCCGCTGGAAAGTACTGATGGCGAGCGCCTTGTAAAG
TCCCCACAATGCTCTAATCCAGGGCTCTGTGTCCAACCCCATCACATAGGGGTTTCT
GTTAAGGAACTCGATTTATATTTGGCATACTTTGTGCATGCAGCAGATTCAAGTCAA
TCTGAAAGTCCCAGCCAGCCAAGTGACGCTGACATTAAGGACCAGCCAGAAAATTA
A
Constructs targeting Nfia, Nfib, and Nfix:
Name: Kozack_3xHA_NFIXdbd_P2A_FLAG_NFIBdbd_P2A_6xHisTag-NFIAdbd_stop (SEQ ID NO: 26):
GCCGCCACCATGTATCCCTACGACGTGCCCGATTACGCTTACCCTTACGATGTGCCT
GACTACGCCTATCCATATGACGTGCCAGACTATGCCTACTCCCCGTACTGCCTCACC
CAGGATGAGTTCCACCCGTTCATCGAGGCACTGCTGCCTCACGTCCGCGCTTTCTCC
TACACCTGGTTCAACCTGCAGGCGCGGAAGCGCAAGTACTTCAAGAAGCATGAAAA
GCGGATGTCGAAGGACGAGGAGCGGGCGGTGAAGGACGAGCTGCTGGGCGAGAAG
CCCGAGATCAAGCAGAAGTGGGCATCCCGGCTGCTGGCCAAGCTGCGCAAGGACAT
CCGGCCCGAGTTCCGCGAGGACTTCGTGCTGACCATCACGGGCAAGAAGCCCCCCT
GCTGCGTGCTCTCCAACCCCGACCAGAAGGGCAAGATCCGGCGGATTGACTGCCTG
CGCCAGGCTGACAAGGTGTGGCGGCTGGACCTGGTCATGGTGATTTTGTTTAAGGGG
ATCCCCCTGGAAAGTACTGATGGGGAGCGGCTCTACAAGTCGCCTCAGTGCTCGAA
CCCCGGCCTGTGCGTCCAGCCACATCACATTGGAGTCACAATCAAAGAACTGGATCT
TTATCTGGCTTACTTTGTCCACACTCCGGAATCCGGACAATCAGATAGTTCAAACCA
GCAAGGAGATGCGGACATCAAACCACTGCCCAACaagcttggaagcggagctactaacttcagcctgctg
aagcaggctggagacgtggaggagaaccctggacctatggagagcgacgagagcggcctgATGGACTACAAAGACCA
TGACGGTGATTATAAAGATCATGACATCGATTACAAGGATGACGATGACAAGATGT
ATTCTCCCATCTGTCTCACTCAGGATGAATTTCACCCATTCATCGAGGCACTTCTTCC
ACATGTCCGTGCAATTGCCTATACTTGGTTCAACCTGCAGGCTCGAAAACGCAAGTA
CTTTAAAAAGCATGAGAAGCGAATGTCAAAGGATGAAGAAAGAGCAGTCAAAGAT
GAGCTTCTCAGTGAAAAGCCTGAAATCAAACAGAAGTGGGCATCCAGGCTCCTTGC
CAAACTGCGCAAAGATATTCGCCAGGAGTATCGAGAGGACTTTGTGCTCACCGTGA
CTGGCAAGAAGCACCCGTGCTGTGTCTTATCCAATCCCGACCAGAAGGGTAAGATTA
GGAGAATCGACTGCCTGCGACAGGCAGACAAAGTCTGGCGTCTGGATCTAGTCATG
GTGATCCTGTTCAAAGGCATCCCCTTGGAAAGTACCGATGGAGAGCGGCTCATGAA
ATCCCCACATTGCACAAACCCAGCACTTTGTGTCCAGCCACATCATATCACAGTATC
AGTTAAGGAGCTTGATTTGTTTTTGGCATACTACGTGCAGGAGCAAGATTCTGGACA
ATCAGGAAGTCCAAGCCACAATGATCCTGCCAAGAATCCTCCAaagcttggaagcggagctacta
acttcagcctgctgaagcaggctggagacgtggaggagaaccctggacctatggagagcgacgagagcggcctgCATCATCAT
CATCATCACagctccggcTATTCTCCGCTCTGTCTCACCCAGGATGAATTTCATCCTTTCA
TCGAAGCACTTCTGCCCCACGTCCGAGCCTTTGCCTACACATGGTTCAACCTGCAGG
CCCGAAAACGAAAATACTTCAAAAAACATGAAAAGCGTATGTCAAAAGAAGAAGA
GAGAGCCGTGAAGGATGAATTGCTAAGTGAAAAACCAGAGGTCAAGCAGAAGTGG
GCATCTCGACTTCTGGCAAAGTTGCGGAAAGATATCCGACCCGAATATCGAGAGGA
TTTTGTTCTTACAGTTACAGGGAAAAAACCTCCATGTTGTGTTCTTTCCAACCCAGAC
CAGAAAGGCAAGATGCGAAGAATTGACTGCCTCCGCCAGGCAGATAAAGTCTGGAG
GTTGGACCTTGTTATGGTGATTTTGTTTAAAGGTATTCCGCTGGAAAGTACTGATGG
CGAGCGCCTTGTAAAGTCCCCACAATGCTCTAATCCAGGGCTCTGTGTCCAACCCCA
TCACATAGGGGTTTCTGTTAAGGAACTCGATTTATATTTGGCATACTTTGTGCATGCA
GCAGATTCAAGTCAATCTGAAAGTCCCAGCCAGCCAAGTGACGCTGACATTAAGGA
CCAGCCAGAAAATTAA

### Example 7

SAG, ABC99, and LY411575 will be tested in pairwise and three-way combination for their ability to stimulate tanycyte-derived neurogenesis. RaxCreER; Sun1-GFP and RaxCreER; Sun1-GFP; Nfia^{lox/lox}; Nfib^{lox/lox}; Nfix^{lox/lox} mice will be used for this study. Mice will be treated with tamoxifen by i.p. injection at P3 and P4. This will induce tanycyte-specific expression of the Sun1-GFP marker gene, and delete Nfia/b/x expression in conditionally mutant mice. Each compound mixture will be injected at 10 mg/kg beginning at either P21 or P60 into control and Nfia/b/x-deficient mice, with treatment continuing for 5 days. Vehicle control injections will be performed in parallel. EdU will be injected daily for three days after the final treatment to measure cell proliferation. Three weeks after the first treatment, mice will be killed and EdU incorporation and immunostaining for the neuronal-specific markers HuC/D and NeuN performed, to determine whether any of these treatments induced proliferation and/or neurogenesis in wildtype and/or Nfia/b/x-deficient tanycytes.

In parallel, we will generate high-titre AAV1-Ef1a-flex-DN construct-RFP virus for the top candidate dominant negative constructs. Based on preliminary experiments in retina, we expect that transactivation domain-deleted dominant-negative constructs simultaneously targeting Nfia, Nfib, and Nfix will be most effective at inducing neurogenic competence in tanycytes, along with the transactivation domain-deleted dominant-negative construct targeting Sox8 and the Sox9-DNA binding domain-KRAB repressor domain fusion. Where possible, multiple dominant-negative constructs will be combined into a single AAV vector using P2A ribosomal frameshifting sequences. We will infect RaxCreER; Sun1-GFP control mice with AAV via intracerebroventricular injection at either P7 or P46, wait 2 weeks, then feed mice tamoxifen-supplemented chow for 2 additional weeks to induce recombination of the flexed inserts. We will then wait an additional two weeks, after which mice will be killed and EdU incorporation and immunostaining for the neuronal-specific markers HuC/D and NeuN performed, to determine whether any of these treatments induced proliferation and/or neurogenesis.

Based on the results of these studies, we will next combine small molecule treatments and dominant-negative AAV constructs to optimize rates of tanycyte-derived neurogenesis, and characterize GFP/RFP-positive cells in detail using scRNA-Seq in order to fully assess the efficiency of cellular reprogramming and to characterize the identity of tancycyte-derived neurons.

### Example 8

We found that that SAG-mediated pharmacological activation of Shh signaling in juvenile mice (P10-12) leads to an increase, including significant increases, in the number of tanycyte-derived neurons. This does not appear to be due to stimulating tanycyte proliferation, but rather to directly promoting differentiation of tanycytes into neurons and/or promoting the survival of newly generated tanycyte-derived neurons.

We also found that that ABC99-mediated activation of Wnt signaling in juvenile mice (P10-12) also promotes generation of tanycyte-derived cells in hypothalamic parenchyma that resemble neurons (still confirming this with immunostaining). This effect is significant, but can be smaller than the effects of SAG.

We determined that intracerebroventricular delivery of the AAV1 serotype can selectively and efficiently infect tanycytes. It was further found that the AAV5 and AAV9 serotypes inefficiently infect tancytyes, while AAV2, AAV6, AAV8, and AAV7m8 do not detectably infect tanycytes.

For the analysis of the effects of activing Shh and Wnt signaling on tanycyte-derived neurogenesis, starting on the indicated day, 3 daily intraperitoneal (i.p.) injections of 10 mg/kg of either the smoothened agonist Sag (stimulate Shh signaling pathway, Sigma) or the Notum antagonist ABC99 (which stimulates the canonical Wnt signaling pathway, Sigma) in postnatal day 10 (P10) control Rax^{CreER}; CAG-lsl-Sun1-GFP mice. Both Sag and ABC99 efficiently cross the blood-brain barrier. Vehicle controls were performed in parallel. To quantify levels of tanycyte proliferation and neurogenesis, 50 mg/kg 5-Ethynyl-2′-deoxyuridine (EdU) was delivered via three once-daily i.p. injections beginning on the first day of treatment. Four days after the final treatment at P16, immunohistochemistry was performed on 25 μm hypothalamic brain sections to quantify the levels of tanycyte proliferation and tanycyte-derived neurogenesis. The number of Edu+/GFP+/HuC/D+ tanycyte-derived neurons in the DMN, VMN, ArcN, and ME of the hypothalamus were then quantified.

Procedure for the Preparation of ABC99: ABC99 (Sigma-Aldrich, #SML2410) was prepared as previously described (except for the fact that a stock solution (5 mg/ml) in ethanol. This stock was sequentially mixed with Tween 80 (Sigma-Aldrich, #P1754), polyethylene glycol, molecular weight 400 (Merck, #91893), and 0.9% NaCl in the ratio of 1:1:1:17. https://www.nature.com/articles/s42568-019-1382-0#Sec2
1) Add 1 ml 100% in DMSO to make Sug/1 ul stock solution
2) Aliquot 1 ml ABC99 stock into 100 ul aliquots
3) Combine 100 ul ABC99, 100 ul Tween-80, 100 ul polyethylene glycol 400, 1700 ul 0.9% NaCl. To make 0.9% NaCl—dissolve 0.9 grams NaCl in 99.1 ml water.
4) Final stock is 0.25 ug/ul—if animal is 10 g then it will need 100 ug (10 mg/kg or 10 ug/g) so inject 400 ul.
5) Vehicle is 100 ul DMSO+100 ul Tween-80, 100 ul polyethylene glycol 400, 0.9% NaCl. To make 0.9% NaCl. Stock concentration now, 0.5 mg/2 mL or 500 ug/2 mL or 0.25 ug/ul
6) Final stock is 0.25 ug/ul—if animal is 10 g then it will need 100 ug (10 mg/kg or 10 ug/g) so inject 400 ul. 40 ul=10 ug
7) Administered EdU 50 mg/kg (stock 5 mg/ml) with a separate i.p injection after, for 3 days.

### Procedure for the Preparation of SAG:

1) Dissolved in 5 mg Sag with 1 ml of 1×PBS.
2) Dilute stock to 1 ug/1 ul stock in 1×PBS that is 200 ul of 5 mg/ml stock into 800 ul of 1× PBS.
3) if animal is 10 g then it will get 10 ug per gram so that is 100 ug/100 ul of 1 ug/1 ul stock.

### Adenovirus-Associated Virus (AAV) Serotypes Tropism in Hypothalamic Tanycytes:

Using the single-cell RNA-sequencing datasets from Nfia/b/x-deficient tanycytes, we have identified multiple transcriptions factors (TFs) as candidate regulators of neurogenic competence in hypothalamic tanycytes. These include TFs such as Ascl1, Sox8, and Sox9. Most of these TFs have known roles in promoting proliferation and neurogenesis in other CNS regions. To selectively drive overexpression of candidate TFs in tanycytes, we have systemically tested the tropism of three of the most common AAV serotypes in hypothalamic tanycytes. The most efficient AAV serotype that selectively infect hypothalamic tanycytes will be used for viral constructs to overexpress candidate TFs of interest.

We have identified the AAV1 serotype to be the most efficiently serotype that infect tanycytes. Our AAV serotype overexpression constructs will be delivered via intracerebroventricular (i.e.v.) injections into the lateral ventricle of P0 CD mice. We will use high-titer AAV1 constructs to overexpressed candidate TFs to promote tanycyte-derived neurogenesis in control tanycytes.

### REFERENCES

1. E. M. Rodríguez, J. L. Blizquez, F. E. Pastor, B. Peláez, P. Peña, B. Peruzzo, P. Amat, Hypothalamic tanycytes: a key component of brain-endocrine interaction. Int. Rev. Cytol. 247, 89-164 (2005).
2. M. Bolborea, N. Dale, Hypothalamic tanycytes: potential roles in the control of feeding and energy balance. Trends Neurosci. 36, 91-100 (2013).
3. D. A. Lee, J. L. Bedont, T. Pak, H. Wang, J. Song, A. Miranda-Angulo, V. Takiar, V.

Charubhumi, F. Balordi, H. Takebayashi, S. Aja, E. Ford, G. Fishell, S. Blackshaw, Tanycytes of the hypothalamic median eminence form a diet-responsive neurogenic niche. Nat. Neurosci. 15, 700-702 (2012).
4. S. C. Robins, I. Stewart, D. E. McNay, V. Taylor, C. Giachino, M. Goetz, J. Ninkovic, N. Briancon, E. Maratos-Flier, J. S. Flier, M. V. Kokoeva, M. Placzek, α-Tanycytes of the adult hypothalamic third ventricle include distinct populations of FGF-responsive neural progenitors. Nat. Commun. 4, 2049 (2013).
5. N. Haan, T. Goodman, A. Najdi-Samiei, C. M. Stratford, R. Rice, E. El Agha, S. Bellusci, M. K. Hajihosseini, Fgf10-expressing tanycytes add new neurons to the appetite/energy-balance regulating centers of the postnatal and adult hypothalamus. J. Neurosci. 33, 6170-6180 (2013).
6. T. Goodman, S. G. Nayar, S. Clare, M. Mikolajczak, R. Rice, S. Mansour, S. Bellusci, M. K. Hajihosseini, Fibroblast growth factor 10 is a negative regulator of postnatal neurogenesis in the mouse hypothalamus. Development. 147 (2020), doi:10.1242/dev.180950.
7. D. A. Lee, S. Yoo, T. Pak, J. Salvatierra, E. Velarde, S. Aja, S. Blackshaw, Dietary and sex-specific factors regulate hypothalamic neurogenesis in young adult mice. Front. Neurosci. 8, 157 (2014).
8. M. Migaud, L. Butrille, M. Batailler, Seasonal regulation of structural plasticity and neurogenesis in the adult mammalian brain: focus on the sheep hypothalamus. Front. Neuroendocrinol. 37, 146-157 (2015).
9. S. Yoo, S. Blackshaw, Regulation and function of neurogenesis in the adult mammalian hypothalamus. Prog. Neurobiol. 170, 53-66 (2018).
10. J. Salvatierra, D. A. Lee, C. Zibetti, M. Duran-Moreno, S. Yoo, E. A. Newman, H. Wang, J. L. Bedont, J. de Melo, A. L. Miranda-Angulo, S. Gil-Perotin, J. M. Garcia-Verdugo, S. Blackshaw, The LIM homeodomain factor Lhx2 is required for hypothalamic tanycyte specification and differentiation. J. Neurosci. 34, 16809-16820 (2014).
11. J. Wan, D. Goldman, Retina regeneration in zebrafish. Curr. Opin. Genet. Dev. 40, 41-47 (2016).
12. M. S. Wilken, T. A. Reh, Retinal regeneration in birds and mice. Curr. Opin. Genet. Dev. 40, 57-64 (2016).
13. A. J. Fischer, R. Bongini, Turning Müller glia into neural progenitors in the retina. Mol. Neurobiol. 42, 199-209 (2010).
14. B. S. Clark, G. L. Stein-O'Brien, F. Shiau, G. H. Cannon, E. Davis-Marcisak, T. Sherman, C. P. Santiago, T. V. Hoang, F. Rajaii, R. E. James-Esposito, R. M. Gronostajski, E. J. Fertig, L. A. Goff, S. Blackshaw, Single-Cell RNA-Seq Analysis of Retinal Development Identifies NFI Factors as Regulating Mitotic Exit and Late-Born Cell Specification. Neuron. 102, 1111-1126.e5 (2019).
15. T. Hoang, J. Wang, P. Boyd, F. Wang, C. Santiago, L. Jiang, S. Yoo, M. Lahne, L. J. Todd, M. Jia, C. Saez, C. Keuthan, I. Palazzo, N. Squires, W. A. Campbell, F. Rajaii, T. Parayil, V. Trinh, D. W. Kim, G. Wang, L. J. Campbell, J. Ash, A. J. Fischer, D. R. Hyde, J. Qian, S. Blackshaw, Gene regulatory networks controlling vertebrate retinal regeneration. Science (2020), doi:10.1126/science.abb8598.
16. D. W. Kim, P. W. Washington, Z. Q. Wang, S. H. Lin, C. Sun, B. T. Ismail, H. Wang, L. Jiang, S. Blackshaw, The cellular and molecular landscape of hypothalamic patterning and differentiation from embryonic to late postnatal development. Nat. Commun. 11, 4360 (2020).
17. R. A. Romanov, E. O. Tretiakov, M. E. Kastriti, M. Zupancic, M. Haring, S. Korchynska, K. Popadin, M. Benevento, P. Rebernik, F. Lallemend, K. Nishimori, F. Clotman, W. D. Andrews, J. G. Parnavelas, M. Farlik, C. Bock, I. Adameyko, T. Hokfelt, E. Keimpema, T. Harkany, Molecular design of hypothalamus development. Nature. 582, 246-252 (2020).
18. T. Pak, S. Yoo, A. L. Miranda-Angulo, H. Wang, S. Blackshaw, Rax-CreERT2 knock-in mice: a tool for selective and conditional gene deletion in progenitor cells and radial glia of the retina and hypothalamus. PLoS One. 9, e90381 (2014).
19. S. Yoo, D. Cha, D. W. Kim, T. V. Hoang, S. Blackshaw, Tanycyte-Independent Control of Hypothalamic Leptin Signaling. Front. Neurosci. 13, 240 (2019).
20. A. Mo, E. A. Mukamel, F. P. Davis, C. Luo, G. L. Henry, S. Picard, M. A. Urich, J. R. Nery, T. J. Sejnowski, R. Lister, S. R. Eddy, J. R. Ecker, J. Nathans, Epigenomic Signatures of Neuronal Diversity in the Mammalian Brain. Neuron. 86, 1369-1384 (2015).
21. R. Chen, X. Wu, L. Jiang, Y. Zhang, Single-Cell RNA-Seq Reveals Hypothalamic Cell Diversity. CellRep. 18, 3227-3241 (2017).
22. F. Langlet, Tanycyte Gene Expression Dynamics in the Regulation of Energy Homeostasis. Front. Endocrinol. 10, 286 (2019).
23. J. N. Campbell, E. Z. Macosko, H. Fenselau, T. H. Pers, A. Lyubetskaya, D. Tenen, M. Goldman, A. M. J. Verstegen, J. M. Resch, S. A. McCarroll, E. D. Rosen, B. B. Lowell, L. T. Tsai, A molecular census of arcuate hypothalamus and median eminence cell types. Nat. Neurosci. 20, 484-496 (2017).
24. E. Matuzelski, J. Bunt, D. Harkins, J. W. C. Lim, R. M. Gronostajski, L. J. Richards, L. Harris, M. Piper, Transcriptional regulation of Nfix by NFIB drives astrocytic maturation within the developing spinal cord. Dev. Biol. 432, 286-297 (2017).
25. S. M. Glasgow, W. Zhu, C. C. Stolt, T.-W. Huang, F. Chen, J. J. LoTurco, J. L. Neul, M. Wegner, C. Mohila, B. Deneen, Mutual antagonism between Sox10 and NFIA regulates diversification of glial lineages and glioma subtypes. Nat. Neurosci. 17, 1322-1329 (2014).
26. D. Vidovic, R. A. Davila, R. M. Gronostajski, T. J. Harvey, M. Piper, Transcriptional regulation of ependymal cell maturation within the postnatal brain. Neural Dev. 13, 2 (2018).
27. B. Kaminskas, T. Goodman, A. Hagan, S. Bellusci, D. M. Ornitz, M. K. Hajihosseini, Characterisation of endogenous players in fibroblast growth factor-regulated functions of hypothalamic tanycytes and energy-balance nuclei. Journal of Neuroendocrinology. 31 (2019), doi:10.1111/jne.12750.
28. G. La Manno, R. Soldatov, A. Zeisel, E. Braun, H. Hochgerner, V. Petukhov, K. Lidschreiber, M. E. Kastriti, P. Lonnerberg, A. Furlan, J. Fan, L. E. Borm, Z. Liu, D. van Bruggen, J. Guo, X. He, R. Barker, E. Sundström, G. Castelo-Branco, P. Cramer, I. Adameyko, S. Linnarsson, P. V. Kharchenko, RNA velocity of single cells. Nature. 560, 494-498 (2018).
29. G. K. Dhoot, M. K. Gustafsson, X. Ai, W. Sun, D. M. Standiford, C. P. Emerson Jr, Regulation of Wnt signaling and embryo patterning by an extracellular sulfatase. Science. 293, 1663-1666 (2001).
30. D. Mizrak, N. S. Bayin, J. Yuan, Z. Liu, R. M. Suciu, M. J. Niphakis, N. Ngo, K. M. Lum, B. F. Cravatt, A. L. Joyner, P. A. Sims, Single-Cell Profiling and SCOPE-Seq Reveal Lineage Dynamics of Adult Ventricular-Subventricular Zone Neurogenesis and NOTUM as a Key Regulator. CellRep. 31, 107805 (2020).
31. R. M. Suciu, A. B. Cognetta 3rd, Z. E. Potter, B. F. Cravatt, Selective Irreversible Inhibitors of the Wnt-Deacylating Enzyme NOTUM Developed by Activity-Based Protein Profiling. ACS Med. Chem. Lett. 9, 563-568 (2018).
32. X. Wang, X.-L. Chou, L. I. Zhang, H. W. Tao, Zona Incerta: An Integrative Node for Global Behavioral Modulation. Trends Neurosci. 43, 82-87 (2020).
33. J. D. Welch, V. Kozareva, A. Ferreira, C. Vanderburg, C. Martin, E. Z. Macosko, Single-Cell Multi-omic Integration Compares and Contrasts Features of Brain Cell Identity. Cell. 177 (2019), pp. 1873-1887.e17.
34. J. Liu, C. Gao, J. Sodicoff, V. Kozareva, E. Z. Macosko, J. D. Welch, Jointly defining cell types from multiple single-cell datasets using LIGER. Nat. Protoc. (2020), doi:10.1038/s41596-020-0391-8.
35. T. Hübschle, E. Thom, A. Watson, J. Roth, S. Klaus, W. Meyerhof, Leptin-induced nuclear translocation of STAT3 immunoreactivity in hypothalamic nuclei involved in body weight regulation. J. Neurosci. 21, 2413-2424 (2001).
36. C. Vaisse, J. L. Halaas, C. M. Horvath, J. E. Darnell Jr, M. Stoffel, J. M. Friedman, Leptin activation of Stat3 in the hypothalamus of wild-type and ob/ob mice but not db/db mice. Nat. Genet. 14, 95-97 (1996).
37. K. Liu, J. Kim, D. W. Kim, Y. S. Zhang, H. Bao, M. Denaxa, S.-A. Lim, E. Kim, C. Liu, I. R. Wickersham, V. Pachnis, S. Hattar, J. Song, S. P. Brown, S. Blackshaw, Lhx6-positive GABA-releasing neurons of the zona incerta promote sleep. Nature. 548, 582-587 (2017).
38. J. A. Dimicco, D. V. Zaretsky, The dorsomedial hypothalamus: a new player in thermoregulation. Am. J. Physiol. Regul. Integr. Comp. Physiol. 292, R47-63 (2007).
39. J. A. DiMicco, B. C. Samuels, M. V. Zaretskaia, D. V. Zaretsky, The dorsomedial hypothalamus and the response to stress: part renaissance, part revolution. Pharmacol. Biochem. Behav. 71, 469-480 (2002).
40. J. Tchieu, E. L. Calder, S. R. Guttikonda, E. M. Gutzwiller, K. A. Aromolaran, J. A. Steinbeck, P. A. Goldstein, L. Studer, NFIA is a gliogenic switch enabling rapid derivation of functional human astrocytes from pluripotent stem cells. Nat. Biotechnol. 37, 267-275 (2019).
41. B. Deneen, R. Ho, A. Lukaszewicz, C. J. Hochstim, R. M. Gronostajski, D. J. Anderson, The transcription factor NFIA controls the onset of gliogenesis in the developing spinal cord. Neuron. 52, 953-968 (2006).
42. Y. Lu, F. Shiau, W. Yi, S. Lu, Q. Wu, J. D. Pearson, A. Kallman, S. Zhong, T. Hoang, Z. Zuo, F. Zhao, M. Zhang, N. Tsai, Y. Zhuo, S. He, J. Zhang, G. L. Stein-O'Brien, T. D. Sherman, X. Duan, E. J. Fertig, L. A. Goff, D. J. Zack, J. T. Handa, T. Xue, R. Bremner, S. Blackshaw, X. Wang, B. S. Clark, Single-Cell Analysis of Human Retina Identifies Evolutionarily Conserved and Species-Specific Mechanisms Controlling Development. Dev. Cell. 53, 473-491.e9 (2020).
43. V. Muthu, H. Eachus, P. Ellis, S. Brown, M. Placzek, Rx3 and Shh direct anisotropic growth and specification in the zebrafish tuberal/anterior hypothalamus. Development. 143, 2651-2663 (2016).
44. T. Shimogori, D. A. Lee, A. Miranda-Angulo, Y. Yang, H. Wang, L. Jiang, A. C. Yoshida, A. Kataoka, H. Mashiko, M. Avetisyan, L. Qi, J. Qian, S. Blackshaw, A genomic atlas of mouse hypothalamic development. Nat. Neurosci. 13, 767-775 (2010).
45. T. S. Corman, S. E. Bergendahl, D. J. Epstein, Distinct temporal requirements for Sonic hedgehog signaling in development of the tuberal hypothalamus. Development. 145 (2018), doi:10.1242/dev.167379.
46. E. A. Newman, D. Wu, M. M. Taketo, J. Zhang, S. Blackshaw, Canonical Wnt signaling regulates patterning, differentiation and nucleogenesis in mouse hypothalamus and prethalamus. Dev. Biol. 442, 236-248 (2018).
47. X. Wang, D. Kopinke, J. Lin, A. D. McPherson, R. N. Duncan, H. Otsuna, E. Moro, K. Hoshijima, D. J. Grunwald, F. Argenton, C.-B. Chien, L. C. Murtaugh, R. I. Dorsky, Wnt signaling regulates postembryonic hypothalamic progenitor differentiation. Dev. Cell. 23, 624-636 (2012).
48. J. E. Lee, S.-F. Wu, L. M. Goering, R. I. Dorsky, Canonical Wnt signaling through Lef1 is required for hypothalamic neurogenesis. Development. 133, 4451-4461 (2006).
49. A. P. Jadhav, S.-H. Cho, C. L. Cepko, Notch activity permits retinal cells to progress through multiple progenitor states and acquire a stem cell property. Proceedings of the National Academy of Sciences. 103 (2006), pp. 18998-19003.
50. J. P. Magnusson, C. Goritz, J. Tatarishvili, D. O. Dias, E. M. K. Smith, O. Lindvall, Z. Kokaia, J. Frisen, A latent neurogenic program in astrocytes regulated by Notch signaling in the mouse. Science. 346, 237-241 (2014).
51. L. Todd, I. Palazzo, N. Squires, N. Mendonca, A. J. Fischer, BMP- and TGFβ-signaling regulate the formation of Müller glia-derived progenitor cells in the avian retina. Glia. 65, 1640-1655 (2017).
52. J. Stipursky, D. Francis, R. S. Dezonne, A. P. Bergamo de Aranjo, L. Souza, C. A. Moraes, F. C. Alcantara Gomes, TGF-β1 promotes cerebral cortex radial glia-astrocyte differentiation in vivo. Front. Cell. Neurosci. 8, 393 (2014).
53. R. A. Poché, Y. Furuta, M.-C. Chaboissier, A. Schedl, R. R. Behringer, Sox9 is expressed in mouse multipotent retinal progenitor cells and functions in Müller glial cell development. J. Comp. Neurol. 510, 237-250 (2008).
54. C. C. Stolt, P. Lommes, E. Sock, M.-C. Chaboissier, A. Schedl, M. Wegner, The Sox9 transcription factor determines glial fate choice in the developing spinal cord. Genes Dev. 17, 1677-1689 (2003).
55. S. Aslanpour, J. M. Rosin, A. Balakrishnan, N. Klenin, F. Blot, G. Gradwohl, C. Schuurmans, D. M. Kurrasch, Ascl1 is required to specify a subset of ventromedial hypothalamic neurons. Development. 147 (2020), p. dev180067.
56. N. L. Jorstad, M. S. Wilken, L. Todd, C. Finkbeiner, P. Nakamura, N. Radulovich, M. J. Hooper, A. Chitsazan, B. A. Wilkerson, F. Rieke, T. A. Reh, STAT Signaling Modifies Ascl1 Chromatin Binding and Limits Neural Regeneration from Müller Glia in Adult Mouse Retina. Cell Rep. 30, 2195-2208.e5 (2020).
57. N. L. Jorstad, M. S. Wilken, W. N. Grimes, S. G. Wohl, L. S. VandenBosch, T. Yoshimatsu, R. O. Wong, F. Rieke, T. A. Reh, Stimulation of functional neuronal regeneration from Müller glia in adult mice. Nature. 548, 103-107 (2017).
58. W. Wang, D. Mullikin-Kilpatrick, J. E. Crandall, R. M. Gronostajski, E. D. Litwack, D. L. Kilpatrick, Nuclear factor I coordinates multiple phases of cerebellar granule cell development via regulation of cell adhesion molecules. J. Neurosci. 27, 6115-6127 (2007).
59. M. Piper, L. Harris, G. Barry, Y. H. E. Heng, C. Plachez, R. M. Gronostajski, L. J. Richards, Nuclear factor one X regulates the development of multiple cellular populations in the postnatal cerebellum. J. Comp. Neurol. 519, 3532-3548 (2011).
60. J. R. Ryu, C. J. Hong, J. Y. Kim, E.-K. Kim, W. Sun, S.-W. Yu, Control of adult neurogenesis by programmed cell death in the mammalian brain. Molecular Brain. 9 (2016), doi:10.1186/s13041-016-0224-4.
61. A. Lafzi, C. Moutinho, S. Picelli, H. Heyn, Tutorial: guidelines for the experimental design of single-cell RNA sequencing studies. Nat. Protoc. 13, 2742-2757 (2018).
62. Y.-C. Hsu, J. Osinski, C. E. Campbell, E. D. Litwack, D. Wang, S. Liu, C. J. Bachurski, R. M. Gronostajski, Mesenchymal nuclear factor I B regulates cell proliferation and epithelial differentiation during lung maturation. Dev. Biol. 354, 242-252 (2011).
63. C. E. Campbell, M. Piper, C. Plachez, Y.-T. Yeh, J. S. Baizer, J. M. Osinski, E. D. Litwack, L. J. Richards, R. M. Gronostajski, The transcription factor Nfix is essential for normal brain development. BMC Dev. Biol. 8, 52 (2008).
64. C. Zeng, F. Pan, L. A. Jones, M. M. Lim, E. A. Griffin, Y. I. Sheline, M. A. Mintun, D. M. Holtzman, R. H. Mach, Evaluation of 5-ethynyl-2′-deoxyuridine staining as a sensitive and reliable method for studying cell proliferation in the adult nervous system. Brain Res. 1319, 21-32 (2010).
65. N. Pentinmikko, S. Iqbal, M. Mana, S. Andersson, A. B. Cognetta 3rd, R. M. Suciu, J. Roper, K. Luopajärvi, E. Markelin, S. Gopalakrishnan, O.-P. Smolander, S. Naranjo, T. Saarinen, A. Juuti, K. Pietiläinen, P. Auvinen, A. Ristimäki, N. Gupta, T. Tammela, T. Jacks, D. M. Sabatini, B. F. Cravatt, Ö. H. Yilmaz, P. Katajisto, Notum produced by Paneth cells attenuates regeneration of aged intestinal epithelium. Nature. 571, 398-402 (2019).
66. S. Yoo, D. Cha, S. Kim, L. Jiang, P. Cooke, M. Adebesin, A. Wolfe, R. Riddle, S. Aja, S. Blackshaw, Tanycyte ablation in the arcuate nucleus and median eminence increases obesity susceptibility by increasing body fat content in male mice. Glia. 68, 1987-2000 (2020).
67. M. Schneeberger, L. Parolari, T. Das Banerjee, V. Bhave, P. Wang, B. Patel, T. Topilko, Z. Wu, C. H. J. Choi, X. Yu, K. Pellegrino, E. A. Engel, P. Cohen, N. Renier, J. M. Friedman, A. R. Nectow, Regulation of Energy Expenditure by Brainstem GABA Neurons. Cell. 178, 672-685.e12 (2019).
68. G. X. Y. Zheng, J. M. Terry, P. Belgrader, P. Ryvkin, Z. W. Bent, R. Wilson, S. B. Ziraldo, T. D. Wheeler, G. P. McDermott, J. Zhu, M. T. Gregory, J. Shuga, L. Montesclaros, J. G. Underwood, D. A. Masquelier, S. Y. Nishimura, M. Schnall-Levin, P. W. Wyatt, C. M. Hindson, R. Bharadwaj, A. Wong, K. D. Ness, L. W. Beppu, H. J. Deeg, C. McFarland, K. R. Loeb, W. J. Valente, N. G. Ericson, E. A. Stevens, J. P. Radich, T. S. Mikkelsen, B. J. Hindson, J. H. Bielas, Massively parallel digital transcriptional profiling of single cells. Nat. Commun. 8, 14049 (2017).
69. T. Stuart, A. Butler, P. Hoffman, C. Hafemeister, E. Papalexi, W. M. Mauck, Y. Hao, M. Stoeckius, P. Smibert, R. Satija, Comprehensive Integration of Single-Cell Data. Cell. 177 (2019), pp. 1888-1902.e21.
70. S. L. Wolock, R. Lopez, A. M. Klein, Scrublet: computational identification of cell doublets in single-cell transcriptomic data. Cell Systems. 8, 231-291 (2019).
71. I. Korsunsky, N. Millard, J. Fan, K. Slowikowski, F. Zhang, K. Wei, Y. Baglaenko, M. Brenner, P.-R. Loh, S. Raychaudhuri, Fast, sensitive and accurate integration of single-cell data with Harmony. Nat. Methods. 16, 1289-1296 (2019).
72. V. Bergen, M. Lange, S. Peidli, F. A. Wolf, F. J. Theis, Generalizing RNA velocity to transient cell states through dynamical modeling. Nat. Biotechnol. (2020), doi:10.1038/s41587-020-0591-3.
73. I. Tirosh, B. Izar, S. M. Prakadan, M. H. Wadsworth 2nd, D. Treacy, J. J. Trombetta, A. Rotem, C. Rodman, C. Lian, G. Murphy, M. Fallahi-Sichani, K. Dutton-Regester, J.-R. Lin, O. Cohen, P. Shah, D. Lu, A. S. Genshaft, T. K. Hughes, C. G. K. Ziegler, S. W. Kazer, A. Gaillard, K. E. Kolb, A.-C. Villani, C. M. Johannessen, A. Y. Andreev, E. M. Van Allen, M. Bertagnolli, P. K. Sorger, R. J. Sullivan, K. T. Flaherty, D. T. Frederick, J. Jané-Valbuena, C. H. Yoon, O. Rozenblatt-Rosen, A. K. Shalek, A. Regev, L. A. Garraway, Dissecting the multicellular ecosystem of metastatic melanoma by single-cell RNA-seq. Science. 352, 189-196 (2016).
74. K. Street, D. Risso, R. B. Fletcher, D. Das, J. Ngai, N. Yosef, E. Purdom, S. Dudoit, Slingshot: cell lineage and pseudotime inference for single-cell transcriptomics. BMC Genomics. 19, 477 (2018).
75. X. Qiu, A. Hill, J. Packer, D. Lin, Y.-A. Ma, C. Trapnell, Single-cell mRNA quantification and differential analysis with Census. Nat. Methods. 14, 309-315 (2017).
76. Y. Zhang, T. Liu, C. A. Meyer, J. Eeckhoute, D. S. Johnson, B. E. Bernstein, C. Nusbaum, R. M. Myers, M. Brown, W. Li, X. S. Liu, Model-based analysis of ChIP-Seq (MACS). Genome Biol. 9, R137 (2008).
77. A. N. Schep, B. Wu, J. D. Buenrostro, W. J. Greenleaf, chromVAR: inferring transcription-factor-associated accessibility from single-cell epigenomic data. Nat. Methods. 14, 975-978 (2017).
78. M. R. Corces, J. M. Granja, S. Shams, B. H. Louie, J. A. Seoane, W. Zhou, T. C. Silva, C. Groeneveld, C. K. Wong, S. W. Cho, A. T. Satpathy, M. R. Mumbach, K. A. Hoadley, A. G. Robertson, N. C. Sheffield, I. Felau, M. A. A. Castro, B. P. Berman, L. M. Staudt, J. C. Zenklusen, P. W. Laird, C. Curtis, Cancer Genome Atlas Analysis Network, W. J. Greenleaf, H. Y. Chang, The chromatin accessibility landscape of primary human cancers. Science. 362 (2018), doi:10.1126/science.aav1898.
79. Z. Shao, Y. Zhang, G.-C. Yuan, S. H. Orkin, D. J. Waxman, MAnorm: a robust model for quantitative comparison of ChIP-Seq data sets. Genome Biol. 13, R16 (2012).
80. S. Heinz, C. Benner, N. Spann, E. Bertolino, Y. C. Lin, P. Laslo, J. X. Cheng, C. Murre, H. Singh, C. K. Glass, Simple Combinations of Lineage-Determining Transcription Factors Prime cis-Regulatory Elements Required for Macrophage and B Cell Identities. Molecular Cell. 38 (2010), pp. 576-589.
81. J. Wang, C. Zibetti, P. Shang, S. R. Sripathi, P. Zhang, M. Cano, T. Hoang, S. Xia, H. Ji, S. L. Merbs, D. J. Zack, J. T. Handa, D. Sinha, S. Blackshaw, J. Qian, ATAC-Seq analysis reveals a widespread decrease of chromatin accessibility in age-related macular degeneration. Nat. Commun. 9, 1364 (2018).
82. H. A. Pliner, J. S. Packer, J. L. McFaline-Figueroa, D. A. Cusanovich, R. M. Daza, D. Aghamirzaie, S. Srivatsan, X. Qiu, D. Jackson, A. Minkina, A. C. Adey, F. J. Steemers, J. Shendure, C. Trapnell, Cicero Predicts cis-Regulatory DNA Interactions from Single-Cell Chromatin Accessibility Data. Mol. Cell. 71, 858-871.e8 (2018).
83. E. Eden, R. Navon, I. Steinfeld, D. Lipson, Z. Yakhini, GOrilla: a tool for discovery and visualization of enriched GO terms in ranked gene lists. BMC Bioinformatics. 10, 48 (2009).
84. F. Supek, M. Bošnjak, N. Škunca, T. Šmuc, REVIGO summarizes and visualizes long lists of gene ontology terms. PLoS One. 6, e21800 (2011).
85. J. E. Frandolig, C. J. Matney, K. Lee, J. Kim, M. Chevee, S.-J. Kim, A. A. Bickert, S. P. Brown, The Synaptic Organization of Layer 6 Circuits Reveals Inhibition as a Major Output of a Neocortical Sublamina. Cell Rep. 28, 3131-3143.e5 (2019).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method of preventing or treating a hypothalamic-regulated behavior in a subject, the method comprising,
administering an effective amount of an agent to the subject, wherein the agent decreases the activity or expression of a nuclear factor I (NFI) gene, and
wherein the hypothalamic-regulated behavior comprises obesity, type II diabetes, a sleep disorder, hypertension, anorexia nervosa, congenital hypothyroidism, neuropsychiatric disorders linked to dysregulation of cortisol, depression, or post-traumatic stress disorder.

2. The method of claim 1, wherein the agent comprises a small molecule, an antibody or fragment thereof, a polypeptide, a nucleic acid molecule, an adeno-associated virus (AAV), protein degraders, or any combination thereof.

3. The method of claim 2, wherein the agent is a nucleic acid molecule that comprises siRNA, miRNA, RNAi, or any combination thereof.

4. The method of claim 2, wherein the agent is AAV that comprises AAV1, AAV2, AAV4, AAV5, AAV6, AAV7, AAV8, AAV9, or AAVDJ8.

5. The method of claim 1, wherein the agent comprises a small molecule.

6. The method of claim 1 wherein the agent is ABC99, SAG (smoothened agonist) and/or LY411575 (gamma secretase inhibitor/Notch antagonist).

7. The method of claim 1 wherein the NFI gene comprises a mammalian NFI gene.

8. The method of claim 1, wherein the decreasing the activity or expression of the NFI gene activates Shh signaling, and/or Wnt signaling.

9. The method of claim 8, further comprising administering a second agent that activates the Shh signaling and/or Wnt signaling.

10. The method of claim 9, wherein the second agent comprises an Shh signaling agonist or a Wnt signaling agonist.

11. The method of claim 9, wherein the second agent comprises a small molecule.

12. The method of claim 11, wherein the second agent small molecule comprises 7-(4-Chlorobenzyl)-1,3-dioxohexahydroimidazo[1,5-a]pyrazin-2(3H)-yl 2,3-dihydro-4Hbenzo[b][1,4]oxazine-4-carboxylate.

13. The method of claim 1, further comprising administering an agent that targets Krüppel-like Factor 2 (Klf2), Krüppel-like Factor 2 (Klf3), V-maf musculoaponeurotic fibrosarcoma oncogene homolog B (Mafb), or combinations thereof.

14. The method of claim 1, further comprising administering an agent that targets Notch homolog 1, translocation-associated (Notch1), Transforming Growth Factor Beta 2 (TGFβ2), Bone Morphogenetic Protein 7 (Bmp7), or combinations thereof.

15. The method of claim 1, further comprising administering a nucleic acid molecule or a vector comprising a nucleic acid molecule having at least a 75% sequence identity to any one or more of SEQ ID NOS: 1-26.

16. The method of claim 1, further comprising administering a nucleic acid molecule or a vector comprising a nucleic acid molecule of any one of SEQ ID NOS: 1-26.

17-21. (canceled)

22. A method for enhancing neurogenic competence in an glial cell in a subject, the method comprising administering an effective amount of an agent to the subject, wherein the agent wherein the agent decreases the activity or expression of a nuclear factor I (NFI) gene, and
wherein neurogenic competence comprises outward radial migration, maturation, and integration into existing hypothalamic circuitry

23-24. (canceled)

25. The method of claim 22, wherein the nucleic acid molecule comprises a sequence identity of at least 75% to at least one of SEQ ID NOS: 1-26.

26. (canceled)

27. A synthetic construct comprising a nucleic acid sequence of any one of SEQ ID NOs: 1-26 or combinations thereof; or
An adeno-associated virus (AAV) comprising a nucleic acid molecule having a sequence identity of at least 75% to at least one of SEQ ID NOS: 1-26.

28-29. (canceled)

30. An isolated cell comprising a nucleic acid molecule having a sequence identity of at least 75% to at least one of SEQ ID NOS: 1-26; or
An isolated cell comprising an adeno-associated virus (AAV) comprising a nucleic acid molecule having a sequence identity of at least 75% to at least one of SEQ ID NOS: 1-26.

31-34. (canceled)


SEQUENCE LISTING (26 sequences)

SEQ ID NO: 1 - DNA, 603 bp, synthetic construct
  source 1..603: mol_type = other DNA
gccgccaccatgtatccctacgacgtgcccgattacgcttacccttacgatgtgcctgactacgcctatccatatgacgtgccagactatgccctggacatgagcgaggcccgctcccagccgccctgcagcccgtccggcaccgccagctccatgtcgcacgtggaggactcggactcggacgcgccgccgtctcccgccggctccgagggcctgggccgcgcgggggtcgcggtggggggcgcccggggcgacccggcggaggcggcggacgagcgcttcccggcctgcatccgcgacgccgtgtcgcaggtgctcaagggctacgactggagtctggtgcccatgccggtgcgcggcggcggcggcggcgcgctcaaagccaagccgcatgtgaagcggcccatgaacgcattcatggtgtgggcgcaggcggcgcgccgcaagctggccgaccagtacccgcacctgcacaacgccgagctcagcaagacgctgggcaagctgtggcgcttgctgagcgagagcgagaagcggcccttcgtggaggaggcagagcgccttcgcgtgcagcacaagaaggaccaccccgactacaagtga

SEQ ID NO: 2 - DNA, 987 bp, synthetic construct
  source 1..987: mol_type = other DNA
gccgccaccatgtatccctacgacgtgcccgattacgcttacccttacgatgtgcctgactacgcctatccatatgacgtgccagactatgccctggacatgagcgaggcccgctcccagccgccctgcagcccgtccggcaccgccagctccatgtcgcacgtggaggactcggactcggacgcgccgccgtctcccgccggctccgagggcctgggccgcgcgggggtcgcggtggggggcgcccggggcgacccggcggaggcggcggacgagcgcttcccggcctgcatccgcgacgccgtgtcgcaggtgctcaagggctacgactggagtctggtgcccatgccggtgcgcggcggcggcggcggcgcgctcaaagccaagccgcatgtgaagcggcccatgaacgcattcatggtgtgggcgcaggcggcgcgccgcaagctggccgaccagtacccgcacctgcacaacgccgagctcagcaagacgctgggcaagctgtggcgcttgctgagcgagagcgagaagcggcccttcgtggaggaggcagagcgccttcgcgtgcagcacaagaaggaccaccccgactacaagtaccagccacggcgcaggaagagcgccaaagccggccacagcgactccgactcgggcgcggagctgggaccccaccctggcggcggtgccgtgtacaaggctgaagcagggcttggagatgggcaccaccatggcgaccacacagggcagacccacgggccgcccaccccgcccaccacccccaagacggagctgcagcaggcgggcgccaagccggagctgaagctggagggacgccggccggtggacagcgggcgccagaacatcgacttcagcaacgtggacatctcggagctcagcagcgaggtcatgggcaccatggacgccttcgacgtccacgagttcgaccagtacctgcccctgggcggccccgccccacccgagtga

SEQ ID NO: 3 - DNA, 1431 bp, synthetic construct
  source 1..1431: mol_type = other DNA
gccgccaccatgtatccctacgacgtgcccgattacgcttacccttacgatgtgcctgactacgcctatccatatgacgtgccagactatgccctggacatgagcgaggcccgctcccagccgccctgcagcccgtccggcaccgccagctccatgtcgcacgtggaggactcggactcggacgcgccgccgtctcccgccggctccgagggcctgggccgcgcgggggtcgcggtggggggcgcccggggcgacccggcggaggcggcggacgagcgcttcccggcctgcatccgcgacgccgtgtcgcaggtgctcaagggctacgactggagtctggtgcccatgccggtgcgcggcggcggcggcggcgcgctcaaagccaagccgcatgtgaagcggcccatgaacgcattcatggtgtgggcgcaggcggcgcgccgcaagctggccgaccagtacccgcacctgcacaacgccgagctcagcaagacgctgggcaagctgtggcgcttgctgagcgagagcgagaagcggcccttcgtggaggaggcagaccgccttcgcgtgcagcacaagaaggaccaccccgactacaagtaccagccacggcgcaggaagagcgccaaagccggccacagcgactccgactcgggcgcggagctgggaccccaccctggcggcggtgccgtgtacaaggctgaagcagggcttggagatgggcaccaccatggcgaccacacagggcagacccacgggccgcccaccccgcccaccacccccaagacggagctgcagcaggcgggcgccaagccggagctgaagctggagggacgccggccggtggacagcgggcgccagaacatcgacttcagcaacgtggacatctcggagctcagcagcgaggtcatgggcaccatggacgccttcgacgtccacgagttcgaccagtacctgcccctgggcggccccgccccacccgagccgggccaggcctatgggggcgcctacttccacgccggggcgtcccccgtgtgggcccacaagagtgccccgtcggcctccgcgtcgcccaccgagacgggtcccccacggccgcacatcaagacggagcagccgagccccggccactacggcgaccagccccgaggctcgcccgactacggttcctgcagcggccagtccagcgccaccccggccgcccccgccggccccttcgccggctcacagggcgactatggcgacctgcaggcctccagctactatggtgcctaccctggctacgcacccggcctctaccagtacccctgcttccactcgccgcgccggccctacgcctcacccctgctcaacggcctggccctgccgcccgcccacagccccaccagtcactgggaccagccggtgtacaccaccctgaccaggccctga

SEQ ID NO: 4 - DNA, 825 bp, synthetic construct
  source 1..825: mol_type = other DNA
gccgccaccatgtatccctacgacgtgcccgattacgcttacccttacgatgtgcctgactacgcctatccatatgacgtgccagactatgccctggacatgagcgaggcccgctcccagccgccctgcagcccgtccggcaccgccagctccatgtcgcacgtggaggactcggactcggacgcgccgccgtctcccgccggctccgagggcctgggccgcgcgggggtcgcggtggggggcgcccggggcgacccggcggaggcggcggacgagcgcttcccggcctgcatccgcgacgccgtgtcgcaggtgctcaagggctacgactggagtctggtgcccatgccggtgcgcggcggcggcggcggcgcgctcaaagccaagccgcatgtgaagcggcccatgaacgcattcatggtgtgggcgcaggcggcgcgccgcaagctggccgaccagtacccgcacctgcacaacgccgagctcagcaagacgctgggcaagctgtggcgcttgctgagcgagagcgagaagcggcccttcgtggaggaggcagagcgccttcgcgtgcagcacaagaaggaccaccccgactacaaggatgccaagagcctgaccgcctggtctagaaccctggtcaccttcaaggacgtgttcgtggacttcacccgggaagagtggaagctgctggatacagcccagcagatcgtgtaccggaacgtgatgctggaaaactacaagaacctggtgtccctgggctaccagctgaccaagcctgacgtgatcctgcggctggaaaagggcgaagaaccttggctggtgtga

SEQ ID NO: 5 - DNA, 1326 bp, synthetic construct
  source 1..1326: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagtag

SEQ ID NO: 6 - DNA, 738 bp, synthetic construct
  source 1..738: mol_type = other DNA
gccgccaccatgtatccctacgacgtgcccgattacgcttacccttacgatgtgcctgactacgcctatccatatgacgtgccagactatgccgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccatag

SEQ ID NO: 7 - DNA, 930 bp, synthetic construct
  source 1..930: mol_type = other DNA
gccgccaccatgtatccctacgacgtgcccgattacgcttacccttacgatgtgcctgactacgcctatccatatgacgtgccagactatgcctccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgtag

SEQ ID NO: 8 - DNA, 795 bp, synthetic construct
  source 1..795: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccatag

SEQ ID NO: 9 - DNA, 795 bp, synthetic construct
  source 1..795: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggaggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccatag

SEQ ID NO: 10 - DNA, 1326 bp, synthetic construct
  source 1..1326: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggaggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagtag

SEQ ID NO: 11 - DNA, 885 bp, synthetic construct
  source 1..885: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggatgccaagagcctgaccgcctggtctagaaccctggtcaccttcaaggacgtgttcgtggacttcacccgggaagagtggaagctgctggatacagcccagcagatcgtgtaccggaacgtgatgctggaaaactacaagaacctggtgtccctgggctaccagctgaccaagcctgacgtgatcctgcggctggaaaagggcgaagaaccttggctggtgtga

SEQ ID NO: 12 - DNA, 663 bp, synthetic construct
  source 1..663: mol_type = other DNA
gccgccaccatgtatccctacgacgtgcccgattacgcttacccttacgatgtgcctgactacgcctatccatatgacgtgccagactatgccaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagagtga

SEQ ID NO: 13 - DNA, 717 bp, synthetic construct
  source 1..717: mol_type = other DNA
gccgccaccatgtatccctacgacgtgcccgattacgcttacccttacgatgtgcctgactacgcctatccatatgacgtgccagactatgcctattctccgctctgtctcacccaggatgaatttcatcctttcatcgaagcacttctgccccacgtccgagcctttgcctacacatggttcaacctgcaggcccgaaaacgaaaatacttcaaaaaacatgaaaagcgtatgtcaaaagaagaagagagagccgtgaaggatgaattgctaagtgaaaaaccagaggtcaagcagaagtgggcatctcgacttctggcaaagttgcggaaagatatccgacccgaatatcgagaggattttgttcttacagttacagggaaaaaacctccatgttgtgttctttccaacccagaccagaaaggcaagatgcgaagaattgactgcctccgccaggcagataaagtctggaggttggaccttgttatggtgattttgtttaaaggtattccgctggaaagtactgatggcgagcgccttgtaaagtccccacaatgctctaatccagggctctgtgtccaaccccatcacataggggtttctgttaaggaactcgatttatatttggcatactttgtgcatgcagcagattcaagtcaatctgaaagtcccagccagccaagtgacgctgacattaaggaccagccagaaaattaa

SEQ ID NO: 14 - DNA, 708 bp, synthetic construct
  source 1..708: mol_type = other DNA
gccgccaccatgtatccctacgacgtgcccgattacgcttacccttacgatgtgcctgactacgcctatccatatgacgtgccagactatgccatgtattctcccatctgtctcactcaggatgaatttcacccattcatcgaggcacttcttccacatgtccgtgcaattgcctatacttggttcaacctgcaggctcgaaaacgcaagtactttaaaaagcatgagaagcgaatgtcaaaggatgaagaaagagcagtcaaagatgagcttctcagtgaaaagcctgaaatcaaacagaagtgggcatccaggctccttgccaaactgcgcaaagatattcgccaggagtatcgagaggactttgtgctcaccgtgactggcaagaagcacccgtgctgtgtcttatccaatcccgaccagaagggtaagattaggagaatcgactgcctgcgacaggcagacaaagtctggcgtctggatctagtcatggtgatcctgttcaaaggcatccccttggaaagtaccgatggagagcggctcatgaaatccccacattgcacaaacccagcactttgtgtccagccacatcatatcacagtatcagttaaggagcttgatttgtttttggcatactacgtgcaggagcaagattctggacaatcaggaagtccaagccacaatgatcctgccaagaatcctccataa

SEQ ID NO: 15 - DNA, 714 bp, synthetic construct
  source 1..714: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaactaa

SEQ ID NO: 16 - DNA, 1356 bp, synthetic construct
  source 1..1356: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggcttttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcccaacggtag

SEQ ID NO: 17 - DNA, 909 bp, synthetic construct
  source 1..909: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccacctaa

SEQ ID NO: 18 - DNA, 1047 bp, synthetic construct
  source 1..1047: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcataa

SEQ ID NO: 19 - DNA, 1170 bp, synthetic construct
  source 1..1170: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccataa

SEQ ID NO: 20 - DNA, 1347 bp, synthetic construct
  source 1..1347: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagtaa

SEQ ID NO: 21 - DNA, 1494 bp, synthetic construct
  source 1..1494: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcccaacggtagcggccagggcaaagtcccggggtcatttttgctaccgccgccgcctccagtggccagacctgtgccccttcctatgcctgattccaaatccaccagcactgccccagacggcgccgccttgactcctccatcaccttaa

SEQ ID NO: 22 - DNA, 936 bp, synthetic construct
  source 1..936: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgatgccaagagcctgaccgcctggtctagaaccctggtcaccttcaaggacgtgttcgtggacttcacccgggaagagtggaagctgctggatacagcccagcagatcgtgtaccggaacgtgatgctggaaaactacaagaacctggtgtccctgggctaccagctgaccaagcctgacgtgatcctgcggctggaaaagggcgaagaaccttggctggtgtaa

SEQ ID NO: 23 - DNA, 1467 bp, synthetic construct
  source 1..1467: mol_type = other DNA
gccgccaccatgtatccctacgacgtgcccgattacgcttacccttacgatgtgcctgactacgcctatccatatgacgtgccagactatgccatgtattctcccatctgtctcactcaggatgaatttcacccattcatcgaggcacttcttccacatgtccgtgcaattgcctatacttggttcaacctgcaggctcgaaaacgcaagtactttaaaaagcatgagaagcgaatgtcaaaggatgaagaaagagcagtcaaagatgagcttctcagtgaaaagcctgaaatcaaacagaagtgggcatccaggctccttgccaaactgcgcaaagatattcgccaggagtatcgagaggactttgtgctcaccgtgactggcaagaagcacccgtgctgtgtcttatccaatcccgaccagaagggtaagattaggagaatcgactgcctgcgacaggcagacaaagtctggcgtctggatctagtcatggtgatcctgttcaaaggcatccccttggaaagtaccgatggagagcggctcatgaaatccccacattgcacaaacccagcactttgtgtccagccacatcatatcacagtatcagttaaggagcttgatttgtttttggcatactacgtgcaggagcaagattctggacaatcaggaagtccaagccacaatgatcctgccaagaatcctccaaagcttggaagcggagctactaacttcagcctgctgaagcaggctggagacgtggaggagaaccctggacctatggagagcgacgagagcggcctgggtaagcctatccctaaccctctcctcggtctcgattctacgtattctccgctctgtctcacccaggatgaatttcatcctttcatcgaagcacttctgccccacgtccgagcctttgcctacacatggttcaacctgcaggcccgaaaacgaaaatacttcaaaaaacatgaaaagcgtatgtcaaaagaagaagagagagccgtgaaggatgaattgctaagtgaaaaaccagaggtcaagcagaagtgggcatctcgacttctggcaaagttgcggaaagatatccgacccgaatatcgagaggattttgttcttacagttacagggaaaaaacctccatgttgtgttctttccaacccagaccagaaaggcaagatgcgaagaattgactgcctccgccaggcagataaagtctggaggttggaccttgttatggtgattttgtttaaaggtattccgctggaaagtactgatggcgagcgccttgtaaagtccccacaatgctctaatccagggctctgtgtccaaccccatcacataggggtttctgttaaggaactcgatttatatttggcatactttgtgcatgcagcagattcaagtcaatctgaaagtcccagccagccaagtgacgctgacattaaggaccagccagaaaattaa

SEQ ID NO: 24 - DNA, 1467 bp, synthetic construct; identical to SEQ ID NO: 23
  source 1..1467: mol_type = other DNA

SEQ ID NO: 25 - DNA, 1467 bp, synthetic construct; identical to SEQ ID NO: 23
  source 1..1467: mol_type = other DNA

SEQ ID NO: 26 - DNA, 2235 bp, synthetic construct
  source 1..2235: mol_type = other DNA
gccgccaccatgtatccctacgacgtgcccgattacgcttacccttacgatgtgcctgactacgcctatccatatgacgtgccagactatgcctactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacaagcttggaagcggagctactaacttcagcctgctgaagcaggctggagacgtggaggagaaccctggacctatggagagcgacgagagcggcctgatggactacaaagaccatgacggtgattataaagatcatgacatcgattacaaggatgacgatgacaagatgtattctcccatctgtctcactcaggatgaatttcacccattcatcgaggcacttcttccacatgtccgtgcaattgcctatacttggttcaacctgcaggctcgaaaacgcaagtactttaaaaagcatgagaagcgaatgtcaaaggatgaagaaagagcagtcaaagatgagcttctcagtgaaaagcctgaaatcaaacagaagtgggcatccaggctccttgccaaactgcgcaaagatattcgccaggagtatcgagaggactttgtgctcaccgtgactggcaagaagcacccgtgctgtgtcttatccaatcccgaccagaagggtaagattaggagaatcgactgcctgcgacaggcagacaaagtctggcgtctggatctagtcatggtgatcctgttcaaaggcatccccttggaaagtaccgatggagagcggctcatgaaatccccacattgcacaaacccagcactttgtgtccagccacatcatatcacagtatcagttaaggagcttgatttgtttttggcatactacgtgcaggagcaagattctggacaatcaggaagtccaagccacaatgatcctgccaagaatcctccaaagcttggaagcggagctactaacttcagcctgctgaagcaggctggagacgtggaggagaaccctggacctatggagagcgacgagagcggcctgcatcatcatcatcatcacagctccggctattctccgctctgtctcacccaggatgaatttcatcctttcatcgaagcacttctgccccacgtccgagcctttgcctacacatggttcaacctgcaggcccgaaaacgaaaatacttcaaaaaacatgaaaagcgtatgtcaaaagaagaagagagagccgtgaaggatgaattgctaagtgaaaaaccagaggtcaagcagaagtgggcatctcgacttctggcaaagttgcggaaagatatccgacccgaatatcgagaggattttgttcttacagttacagggaaaaaacctccatgttgtgttctttccaacccagaccagaaaggcaagatgcgaagaattgactgcctccgccaggcagataaagtctggaggttggaccttgttatggtgattttgtttaaaggtattccgctggaaagtactgatggcgagcgccttgtaaagtccccacaatgctctaatccagggctctgtgtccaaccccatcacataggggtttctgttaaggaactcgatttatatttggcatactttgtgcatgcagcagattcaagtcaatctgaaagtcccagccagccaagtgacgctgacattaaggaccagccagaaaattaa